SEQUENCE LISTING (16 sequences)

SEQ ID NO: 1 - DNA, 23 bp, synthetic construct
  source 1..23: mol_type = other DNA
cgatgcatagggacaaagagtgg

SEQ ID NO: 2 - DNA, 23 bp, synthetic construct
  source 1..23: mol_type = other DNA
ggaaagagaactgtcggagtggg

SEQ ID NO: 3 - DNA, 28 bp, synthetic construct
  source 1..28: mol_type = other DNA
ggctggaaagagaactgtcggagtgggt

SEQ ID NO: 4 - DNA, 23 bp, synthetic construct
  source 1..23: mol_type = other DNA
agtgggtgaatccagccaggagg

SEQ ID NO: 5 - DNA, 23 bp, synthetic construct
  source 1..23: mol_type = other DNA
ccctgctctcatcaagaccgggg

SEQ ID NO: 6 - protein, 96 aa, synthetic construct
ALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 7 - protein, 197 aa, synthetic construct
VIVVSTVAFLIYHFYFHLILIAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT

SEQ ID NO: 8 - protein, 120 aa, synthetic construct [sequence omitted]

SEQ ID NO: 9 - protein, 113 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAP

SEQ ID NO: 10 - protein, 123 aa, synthetic construct
QVQLLQSGAAVTKPGASVRVSCEASGYNIRDYFIHWWRQAPGQGLQWVGWINPKTGQPNNPRQFQGRVSLTRHASWDFDTFSFYMDLKALRSDDTAVYFCARQRSDYWDFDVWGSGTQVTVSS

SEQ ID NO: 11 - protein, 99 aa, synthetic construct
DIQMTQSPSSLSASVGDTVTITCQANGYLNWYQQRRGKAPKLLIYDGSKLERGVPSRFSGRRWGQEYNLTINNLQPEDIATYFCQVYEFVVPGTRLDLK

SEQ ID NO: 12 - protein, 483 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGAGSGASWSHPQFEKGASGGSGGASWSHPQFEKGASGGSGGSGGASWSHPQFEKGASGGAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT

SEQ ID NO: 13 - protein, 403 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGAGSGASWSHPQFEKGASGGSGGASWSHPQFEKGASGGSGGSGGASWSHPQFEKGASGGALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 14 - protein, 473 aa, synthetic construct
DIQMTQSPSSLSASVGDTVTITCQANGYLNWYQQRRGKAPKLLIYDGSKLERGVPSRFSGRRWGQEYNLTINNLQPEDIATYFCQVYEFVVPGTRLDLKRGGGGSGGGGSGGGGSQVQLLQSGAAVTKPGASVRVSCEASGYNIRDYFIHWWRQAPGQGLQWVGWINPKTGQPNNPRQFQGRVSLTRHASWDFDTFSFYMDLKALRSDDTAVYFCARQRSDYWDFDVWGSGTQVTVSSGAGSGASWSHPQFEKGASGGSGGASWSHPQFEKGASGGSGGSGGASWSHPQFEKGASGGAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT

SEQ ID NO: 15 - protein, 393 aa, synthetic construct
DIQMTQSPSSLSASVGDTVTITCQANGYLNWYQQRRGKAPKLLIYDGSKLERGVPSRFSGRRWGQEYNLTINNLQPEDIATYFCQVYEFVVPGTRLDLKRGGGGSGGGGSGGGGSQVQLLQSGAAVTKPGASVRVSCEASGYNIRDYFIHWWRQAPGQGLQWVGWINPKTGQPNNPRQFQGRVSLTRHASWDFDTFSFYMDLKALRSDDTAVYFCARQRSDYWDFDVWGSGTQVTVSSGAGSGASWSHPQFEKGASGGSGGASWSHPQFEKGASGGSGGSGGASWSHPQFEKGASGGALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 16 - protein, 451 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGAGSGASWSHPQFEKGASGGSGGASWSHPQFEKGASGGSGGSGGASWSHPQFEKGASGGALLVIPVVMGILITIFGVFLYIKKVVKKPKDNEILPPAARRQDPQEMEDYPGHNTAAPVQETLHGCQPVTQEDGKESRISVQERQVTDSIALRPLVDKDDGKAGMEEDHTYEGLNIDQTATYEDIVTLRTGEVKWSVGEHPGQE

(19) United States
(12) Patent Application Publication
(10) Pub. No.: US 2025/0099503 A1
(43) Pub. Date: Mar. 27, 2025

(54) ENGINEERING B CELLS TO EXPRESS CHIMERIC ANTIGEN RECEPTORS (CARS) AND USES THEREOF FOR T CELL INDEPENDENT ACTIVATION

(71) Applicant: Ramot at Tel-Aviv University Ltd., Tel Aviv (IL)

(72) Inventors: Adi BARZEL, Tel-Aviv (IL); Alessio D. NAHMAD, Tel-Aviv (IL); Rashad TARADI, Tel-Aviv (IL)

(73) Assignee: Ramot at Tel-Aviv University Ltd., Tel Aviv (IL)

(21) Appl. No.: 18/729,713

(22) PCT Filed: Jan. 25, 2023
(86) PCT No.: PCT/IL2023/050086

§ 371 Date: Jul. 17, 2024

Related U.S. Application Data
(60) Provisional application No. 63/302,827, filed on Jan. 25, 2022.

(51) Int. Cl. A61K 35/17; A61K 38/00; A61K 39/00; A61P 35/00; C07K 14/705; C07K 14/715; C07K 14/725; C07K 16/10; C07K 16/32; C12N 5/0781

(52) U.S. Cl. CPC A61K 35/17; A61K 40/13; A61K 40/31; A61K 40/4205; A61K 40/46; A61P 35/00; C07K 14/7051; C07K 14/70578; C07K 14/7155; C07K 14/7156; C07K 16/1063; C07K 16/32; C12N 5/0635; A61K 38/00; C07K 2319/02; C07K 2319/03; C12N 2510/00; C12N 2750/14143

(57) ABSTRACT

The present disclosure describes a chimeric antigen receptors (CAR) that allow a cell of the B cell lineage to undergo antigen-induced activation independent of a cell of T cell lineage, compositions thereof, and method of use thereof for genetically modifying a cell of the B cell lineage or treating a subject suffering from a pathological disorder.

### SEQUENCE LISTING STATEMENT

The instant application contains a Sequence Listing, which has been submitted electronically in XML format and is hereby incorporated by reference in its entirety. The XML format copy, created on Jan. 24, 2023 is named P-614156-PC_SL (1).xml and is 18 kilobytes in size.

### FIELD OF THE INVENTION

The invention relates to gene therapy, specifically, immunotherapy. More specifically, the invention relates to chimeric antigen receptors for B cells (CAR-B), methods and compositions for engineering B cells to express for T-cell independent activation and uses thereof in immunotherapy.

### BACKGROUND ART

[1] WO 2020/053865 A1;
[2] Nahmad A D, et. al., (2020) Nat Commun 11, 5851. doi.org/10.1038/s41467-020-19649-1;
[3] Luo X M, et. al., (2009) Blood. 113(7):1422-1431.
[4] Huang D, et. al., (2020) Nat. Commun. 10.1038/s41467-020-19650-8.
[5] Hartweger H, et al., (2019) J. Exp. Med. 216:1301-1310. doi:10.1084/jem. 20190287.
[6] Moffett H F, et. al., (2019) Sci. Immunol. 4, 35.
[7] Greiner V, et. al., (2019) Iscience. 12:369-378. doi: 10.1016/j.isci.2019.01.032.
[8] Voss J E, et. al., (2019) Elife 10.7554/eLife.42995.
[9] Hung K L, et. al., (2018) Mol Ther. 26:456-67.
[10] Pesch T, et. al., (2019) Front. Immunol. 10:2630. doi:10.3389/fimmu.2019.02630

Acknowledgement of the above references herein is not to be inferred as meaning that these are in any way relevant to the patentability of the presently disclosed subject matter.

### BACKGROUND OF THE INVENTION

B cell engineering is a rising field with applications in multiple medical conditions.

The inventors and others have previously demonstrated engineering of B cell's antibody (ABs) genes for expression as predefined secreted antibodies, as well as a functional cell surface B cell receptor (BCR) for predefined antigens. More specifically, the inventors previously demonstrated engineering of B cells for the expression of anti-HIV broadly neutralizing antibodies (HIV-bNAbs). Upon binding the HIV antigen, the B cells differentiate into memory cells and antibody-secreting plasma cells and undergo affinity maturation, constituting a potential one-shot treatment for HIV infections. Luo et al. engineered human hematopoietic stem/progenitor cells to produce an anti-HIV Ab upon in vitro maturation to B cells. Huang D, et. al., and Hartweger H, et al., also demonstrated that HIV bNAbs-engineered primary mouse B cells can be adoptively transferred and vaccinated in immunocompetent mice resulting in expansion of durable bNAb memory and long-lived plasma cells, conferring protection against infection.

Similarly, other studies have also highlighted the utility of genome editing of B cells to secrete therapeutic proteins as vaccination approach against different photogenes.

Pesch T, et. al., engineered B cells to carry synthetic B cell antigen receptors, thereby producing chimeric B cell receptors (CBCR) that exhibit surface expression and antigen recognition capability. However, the CBCRs disclosed therein include signal transduction domains derived specifically from the B cell receptor (BCR) complex, and B cell activation is therefore still dependent on T cell help.

B cells naturally express B cell receptors (BCRs) on their cell membranes. Upon specific binding of an appropriate antigen to the BCR, the B cell may be activated, and it may differentiate into an antibody secreting plasma cell. Antigen-induced activation is a tightly regulated process, which takes place mainly in germinal centers, and may result in affinity maturation, class switch recombination and memory retention. Importantly, it should be noted that antigen-induced B cell activation in the germinal centers requires the participation of dedicated T cells and dendritic cells.

Moreover, in all previous publications, the in vivo antigen-induced activation of the engineered B cells was dependent, or would depend, on T cell help. This dependency becomes a limiting factor in various conditions, including, for example, targeting a self-antigen in cases where T cell help may be absent because of immune tolerance. Another example includes patients with various medical conditions, that may be naturally immunocompromised, or patients with various medical conditions that may receive immunosuppressive or immunoablating treatments. Still further, dependency on T cell activation is not desired also in medical conditions such as cancer, that may exhibit active immunosuppression and immune exclusion.

Therefore, there is an unmet need to engineer B cells capable of full activation without needing the help of T cells. Such cells would be able to mimic intracellular signal transduction for activation by T cells, thereby executing T-cell dependent B cell response without being dependent on activation signals coming from T cells and on interacting with T cell independent activation. The engineering of a CAR molecule capable of T cell independent activation may be applied in various pathological conditions such as cancer, autoimmune disease, congenital disorders, infectious diseases, allergies, cardiovascular diseases, neurodegenerative diseases, diabetes, drug addictions and more.

These unmet needs are addressed by the present disclosure.

### SUMMARY OF THE INVENTION

A first aspect of the present disclosure relates to a chimeric antigen receptor (CAR) molecule that allows a cell of the B cell lineage to undergo antigen-induced activation independent of a cell of the T cell lineage. More specifically, the CAR of the present disclosure comprises the following components: (i) at least one target-binding domain; (ii) at least one transmembrane domain; and (iii) at least one signal transduction domain. It should be understood that this signal transduction domain is capable of relaying, and transducing signals for T cell independent activation.

A further aspect of the present disclosure relates to a system comprising:
(a) at least one CAR molecule; and (b) at least one effector protein; or (c) at least one nucleic acid molecule comprising at least one nucleic acid sequence encoding at least one of (a), (b) or (a) and (b), or any or any cassette, vector or vehicle comprising the at least one nucleic acid molecule. More specifically, the CAR molecule of the disclosed system allows a cell of the B cell lineage to undergo antigen-induced activation independent of a cell of the T cell lineage. In some embodiments, the CAR molecule of the disclosed system comprises the following components: (i) at least one target-binding domain; (ii) at least one transmembrane domain; and (iii) at least one signal transduction domain. It should be noted that the transduction domain of the disclosed CAR molecule is capable of relaying signals for T cell independent activation. More specifically, the transduction domain of the CAR molecule of the disclosed system is characterized in its ability to induce immune activation that is not dependent on T cells.

A further aspect of the present disclosure relates to a nucleic acid molecule comprising at least one nucleic acid sequence encoding at least one CAR that allows a cell of the B cell lineage to undergo antigen-induced activation independent of a cell of the T cell lineage, or any cassette, vector or vehicle comprising said nucleic acid molecule. In some embodiments the CAR encoded by the nucleic acid sequence of the present disclosure comprises the following components: (i) at least one target-binding domain; (ii) at least one transmembrane domain; and (iii) at least one signal transduction domain. It should be understood that this signal transduction domain is capable of relaying, and transducing signals for T cell independent activation.

A further aspect of the present disclosure relates to a gene editing system comprising: (i) at least one nucleic acids molecule as defined by the present invention, or any cassette, vector or vehicle comprising said at least one nucleic acid molecule; and (ii) at least one gene editing component or a nucleic acid sequence encoding the gene editing component.

A further aspect of the present disclosure relates to a genetically engineered cell of the B cell lineage expressing at least one CAR molecule, or any population of cells comprising at least one of the genetically modified cell/s of the present disclosure. The engineered cell of the present disclosure is capable of undergoing antigen-induced activation independent of a cell of the T cell lineage. In yet some further embodiments, the CAR molecule of the engineered cell/s of the present disclosure comprises: (i) at least one target-binding domain; (ii) at least one transmembrane domain; and (iii) at least one signal transduction domain. It should be noted that the signal transduction domain of the CAR of the engineered cell/s of the present disclosure is capable of relaying signals for T cell independent activation.

A further aspect of the present disclosure relates to a pharmaceutical composition comprising at least one CAR molecule that allows a cell of the B cell lineage to undergo antigen-induced activation independent of a cell of the T cell lineage, or any system comprising the CAR molecule disclosed herein, any nucleic acid molecule comprising at least one nucleic acid sequence encoding the CAR molecule, or any, cassette, vector, vehicle or gene editing system comprising the nucleic acid molecule, any cell expressing the CAR molecule of the present disclosure, or any genetically engineered B cell expressing the CAR molecule or population of cells comprising at least one the genetically engineered B cell disclosed herein. In some embodiments, the CAR molecule comprises the following components: (i) at least one target-binding domain; (ii) at least one transmembrane domain; and (iii) at least one signal transduction domain. It should be understood that this signal transduction domain is capable of relaying, and transducing signals for T cell independent activation. In some optional embodiments, the composition disclosed herein may further comprise at least one of pharmaceutically acceptable carrier/s, diluent/s, excipient/s and additive/s.

A further aspect of the resent disclosure relates to a method for genetic engineering of a cell of the B cell lineage to express at least one CAR molecule that allows the cell to undergo antigen-induced activation independent of a cell of the T cell lineage. The method comprising the step of contacting the cell of the B lineage with an effective amount of at least one nucleic acid molecule comprising at least one nucleic acid sequence encoding said CAR molecule or a system comprising the CAR, or any cassette, vehicle, vector or gene editing system comprising the nucleic acid molecule, or with a composition thereof. In some embodiments, the CAR of the disclosed methods comprises the following components: (i) at least one target-binding domain; (ii) at least one transmembrane domain; and (iii) at least one signal transduction domain. It should be understood that this signal transduction domain is capable of relaying, and transducing signals for T cell independent activation.

A further aspect of the present disclosure relates to a method for genetic engineering of a cell of the B cell lineage to express at least one CAR molecule that allows a cell of the B cell lineage to undergo antigen-induced activation independent of a cell of the T cell lineage in a subject in need thereof. In some embodiments, the disclosed method comprises the step of administering to the subject an effective amount of at least one nucleic acid molecule comprising at least one nucleic acid sequence encoding the CAR molecule or a system comprising the CAR, or any cassette, vehicle, vector or gene editing system comprising the nucleic acid molecule, or a composition thereof. In some embodiments, the CAR of the disclosed methods comprises the following components: (i) at least one target-binding domain; (ii) at least one transmembrane domain; and (iii) at least one signal transduction domain. It should be understood that this signal transduction domain is capable of relaying, and transducing signals for T cell independent activation.

A further aspect of the present disclosure relates to an effective amount of at least one nucleic acid molecule comprising at least one nucleic acid sequence encoding at least one CAR or a system comprising said CAR, or any cassette, vehicle, vector or gene editing system comprising the nucleic acid molecule, or a composition thereof for use in a method for genetic engineering of a cell of the B cell lineage to express said CAR molecule in a subject in need thereof. In some embodiments, the CAR molecule comprises the following components: (i) at least one target-binding domain; (ii) at least one transmembrane domain; and (iii) at least one signal transduction domain. It should be understood that this signal transduction domain is capable of relaying, and transducing signals for T cell independent activation.

A further aspect of the present disclosure relates to a method for treating, preventing, ameliorating, inhibiting or delaying the onset of a pathologic disorder in a mammalian subject, said method comprising the step of administering to said subject an effective amount of at least one of: (a) at least one nucleic acid molecule encoding least one CAR molecule that allows a cell of the B cell lineage to undergo antigen-induced activation independent of a cell of the T cell, or at least one system comprising said CAR; (b) at least one cassette, vector vehicle or gene editing system comprising said nucleic acid molecule of (a); (c) at least one cell expressing said CAR or a system comprising said CAR, or a population of said cells; and (d) a composition comprising at least one of (a), (b) and (c). In some embodiments, the CAR molecule used by the methods of the present disclosure comprises the following components: (i) at least one target-binding domain; (ii) at least one transmembrane domain; and (iii) at least one signal transduction domain. It should be understood that this signal transduction domain is capable of relaying, and transducing signals for T cell independent activation.

These and other aspects of the present disclosure will become apparent by the hand of the following disclosure.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1B: General representation of an engineered CAR BCR B cell capable of T cell independent activation

FIG. 1A: Schematic representation of Donor AAV expressing an antigen specific CAR with T cell independent intracellular signaling domain and an antigen specific BCR (CAR+BCR), following integration in the IgH locus.

FIG. 1B: Schematic representation of an engineered B cell expressing the transgenic antigen receptors, CAR-B and BCR, on its cell surface.

FIG. 2A-2G: Engineering of B cells to express 3BNC117 CAR and BCR and in-vitro activation

FIG. 2A: Experimental scheme. Splenic B lymphocytes are collected from donor mice and activated with LPS and IL4. The following day, cells are engineered using CRISPR-Cas9 RNPs and AAV donors. Cells are rested in the presence of LPS for two additional days before acquisition in flow cytometry. Finally, the culture is supplemented with gp120 for activation and concentration is monitored for two additional days.

FIG. 2B: Donor AAV expressing a 3BNC117 CAR and BCR (CAR+BCR) for integration into the IgH locus.

FIG. 2C: Representative flow cytometry of the engineering efficiency, as indicated by gp120 binding and anti-idiotype to 3BNC117 staining. Control represents cells non-engineered. BCR represent cells engineered to express only the 3BNC117 BCR.

FIG. 2D: Concentration of cells tracked over time for either BCR or CAR+BCR engineered cells.

FIG. 2E: Donor AAV expressing a 3BNC117 CAR and Trastuzumab BCR following integration in the IgH locus.

FIG. 2F: Representative flow cytometry of the engineering efficiency, as indicated by Her binding and anti-idiotype to 3BNC117 staining.

FIG. 2G: Fold expansion of cells tracked over time for cells engineered as in E. and grown in gp120 two days following Flow Cytometry.

### DETAILED DESCRIPTION OF THE INVENTION

A first aspect of the present disclosure relates to a chimeric antigen receptor (CAR) molecule that allows a cell of the B cell lineage to undergo antigen-induced activation independent of a cell of the T cell lineage. More specifically, the CAR of the present disclosure comprises the following components: (i) at least one target-binding domain; (ii) at least one transmembrane domain; and (iii) at least one signal transduction domain. It should be understood that this signal transduction domain is capable of relaying, and transducing signals for T cell independent activation.

In some embodiments, the at least one target binding domain of the CAR molecule of the present disclosure comprises: (i) at least one target-recognition element. In some additional or alternative embodiments the at least one target binding domain may further, or alternatively comprise (ii) at least one adaptor component that recognizes and binds at least one tagged target-recognition element.

In yet some further embodiments, the target-recognition element of the CAR molecule of the present disclosure comprises at least one antibody or any antigen-binding fragment/s, portion/s or chimera/s thereof.

In certain embodiments, disclosed herein is a chimeric antigen receptor (CAR) that allows a cell of the B cell lineage to undergo antigen-induced activation independent of a cell of the T cell lineage, wherein said CAR comprises:
at least one target-binding domain comprising at least one target-recognition element, wherein said target is at least one antigen associated with a pathologic disorder and said pathologic disorder is at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition;
at least one transmembrane domain; and
at least one signal transduction domain, wherein said signal transduction domain is not derived from a B cell receptor and is capable of relaying signals for T cell independent activation; and
   wherein said cell of the B cell lineage is at least one of B cell precursor, naïve B cell, and activated B cell of all phenotypes.

It should be understood that in some embodiments, the binding moieties of the target-recognition element of the CAR molecule of the present disclosure may comprise a single antigen specificity (monospecific), or alternatively, two, three or more antigen specificities. Thus, in certain embodiments, the target binding moiety of the CAR molecule of the present disclosure, may comprise antibodies or antigen binding fragment thereof that are directed to two o more antigens (e.g., bi-specific, or tri-specific antibodies).

In some embodiments, disclosed herein is a CAR wherein said at least one target-binding domain further comprises at least one adaptor component that recognizes and binds at least one tagged target-recognition element.

In some embodiments, the adaptor component comprises at least one moiety that specifically recognizes and binds at least one tag of said tagged target-recognition element. In some embodiments, a “tagged target-recognition element” may encompass an antibody, or a receptor or a DARPin (designed ankyrin repeat proteins), etc.) that are tagged so that they may be bound by the adapter. The tagged recognition element may be provided to the patient as a component of an engineered CAR B cell described herein, wherein, the CAR is endowed with the adapter, or in an alternative embodiment, the tagged target recognition element can be provided after the administration of the engineered CAR B cells.

According to such specific embodiments, the CAR molecule of the present disclosure may be adapted for various target recognition elements that are tagged by a tag recognized and by the adaptor component, thereby forming a recognition pair. These CAR molecules may be also indicated herein as universal CARs.

In some embodiments, a CAR molecule disclosed herein comprising an at least one target binding domain comprises (i) at least one target-recognition element and/or (ii) at least one adaptor component that recognizes and binds at least one tagged target-recognition element. In some embodiments, a CAR molecule disclosed herein comprising an at least one target binding domain comprises at least one target-recognition element. In some embodiments, a CAR molecule disclosed herein comprising an at least one target binding domain comprises (i) at least one target-recognition element and (ii) at least one adaptor component that recognizes and binds at least one tagged target-recognition element. In some embodiments, a CAR molecule disclosed herein comprising an at least one target binding domain comprises at least one adaptor component that recognizes and binds at least one tagged target-recognition element. In some embodiments, a CAR molecule disclosed herein comprising an at least one target binding domain comprises (i) at least one target-recognition element or (ii) at least one adaptor component that recognizes and binds at least one tagged target-recognition element.

In some embodiments, the recognition pair may include the biotin/avidin affinity pair. For example, the target recognition component may be tagged by biotin and attached to adaptor component of the disclosed CAR that comprises Streptavidin. It should be however apricated that any other binding pairs are applicable for this purpose, for example, leucine zipper adaptor (zipCAR, and zipFv), Peptide neo-epitope (PNE) and anti-PNE, fluorescein (FITC) and anti-FITC, 10 amino acids (5B9 tag) and anti 5B9, FLAG, HA, SpyTag/SpyCatcher, Leucine zipper, SNAP-tag, CLIP-tag, Halo-tag, SpyTag, SnoopTag, Isopep-tag, and the like.

In some embodiments, the target targeted by the target binding domain of the disclosed CAR is at least one antigen associated with a pathologic disorder.

Still further, such target is an antigen associated with at least one pathologic disorder that may be in some embodiments, at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition.

In more specific embodiments, the proliferative disorder is cancer. Accordingly, the target-binding domain specifically recognizes and binds at least one target, specifically an antigen associated with, expressed by or comprised within a tumor tissue (or tumor microenvironment, TME), or by at least one tumor cell. Thus, in some embodiments, the target is at least one tumor associated antigen (TAA).

As discussed herein after, various TAA's are applicable in the present disclosure, specifically, any TAA disclosed by the invention. In yet some further specific embodiments, the TAA is the human epidermal growth factor receptor 2 (HER2/neu) or any fragment thereof. Thus, in some embodiments, the target binding domain of the CAR of the present disclosure specifically recognizes and binds HER2/neu.

In more specific embodiments, the target-recognition element of the disclosed CAR comprises at least one antibody or any antigen-binding fragment/s, portion/s or chimera/s thereof that specifically recognizes and binds HER2/neu.

Still further, in some non-limiting embodiments, an anti-HER2/neu antibody applicable in the present invention is the monoclonal antibody Trastuzumab, also known as Herceptin, Herzuma or Ogivri. Still further, in some embodiments, the disclosed CAR may comprise at least one of the variable heavy chain (VH) and/or the variable light chain (VL) of the Trastuzumab antibody. More specifically, in some embodiments the VH of Trastuzumab may comprise the amino acid sequence as denoted by SEQ ID NO: 8. Still further, in some embodiments the VL of Trastuzumab may comprise the amino acid sequence as denoted by SEQ ID NO: 9.

In yet some further specific and non-limiting embodiments, the TAA is the human cytokine receptor glycoprotein 130 (gp130) or any fragment thereof. Thus, in some embodiments, the target binding domain of the CAR of the present disclosure specifically recognizes and binds gp130.

In more specific embodiments, the target-recognition element of the disclosed CAR comprises at least one antibody or any antigen-binding fragment/s, portion/s or chimera/s thereof that specifically recognizes and binds gp130.

In yet some further specific and non-limiting embodiments, the TAA is the Melanoma Antigen E (MAGE) or any fragment thereof. Thus, in some embodiments, the target binding domain of the CAR of the present disclosure specifically recognizes and binds MAGE any other melanoma related antigens.

In more specific embodiments, the target-recognition element of the disclosed CAR comprises at least one antibody or any antigen-binding fragment/s, portion/s or chimera/s thereof that specifically recognizes and binds MAGE or any other melanoma related antigens.

In yet some alternative embodiments, the pathologic disorder is an infectious disease caused by a viral pathogen. Accordingly, the target-binding domain of the disclosed CAR molecule, specifically recognizes and binds at least one target, specifically an antigen associated with, expressed by or comprised within a tissue and/or cell affected by a viral pathogen.

In some embodiments, the viral pathogen is the human immunodeficiency virus (HIV).

Still further, in some embodiments, wherein said target-recognition element comprises at least one antibody or any antigen-binding fragment/s, portion/s or chimera/s thereof that specifically recognizes and binds gp120. In some embodiments, the anti-gp120 antibodies are broadly neutralizing HIV-1 antibodies (bNAb). In more specific embodiments, the neutralizing HIV-1 antibody is 3BNC117. In yet some further embodiments, the VH of the 3BNC117 antibody may comprise the amino acid sequence as denoted by SEQ ID NO: 10. Still further, in some embodiments the VL of 3BNC117 may comprise the amino acid sequence as denoted by SEQ ID NO: 11.

In some embodiments, a CAR comprising at least one target-binding domain comprising at least one target-recognition element associated with a pathological disorder, the pathological disorder comprises a proliferative disorder and the proliferative disorder is cancer and the target-binding domain specifically recognizes at least one tumor associated antigen (TAA), and the TAA is the human epidermal growth factor receptor 2 (HER2/neu) or any fragment thereof; or when the pathological disorder comprises an infectious disease caused by a pathogen, the pathogen is the human immunodeficiency virus (HIV) and the target-binding domain specifically recognizes a gp120 HIV antigen.

In some embodiments of a CAR disclosed herein, when the disorder is cancer, the target-recognition element comprises at least one antibody or any antigen-binding fragment/s, portion/s or chimera/s thereof that specifically recognizes and binds HER2/neu; or when the disorder is an infectious disease caused by a pathogen, and the pathogen is HIV, the target-recognition element comprises at least one antibody or any antigen-binding fragment/s, portion/s or chimera/s thereof that specifically recognizes and binds gp120.

Still further, in some embodiments, the target-recognition domain of the CAR molecule of the present disclosure is part of the extracellular domain of the molecule. In yet some further embodiments, the extracellular domain of the CAR molecule of the present disclosure my comprise at least one additional components or domains. More specifically, further components that may be added to the extracellular domain may comprise for example, at least one spacer domain comprising at least one tag or labeling moiety. Such labeling moiety may comprise for example, Strep-II, HA-tag and the like. In yet some further embodiments, the extracellular domain of the disclosed CAR molecule may comprise at least one hinge region. The hinge region, as used herein, is a flexible amino acid stretch/structure, which may in some embodiments link between the target-binding domain and the transmembrane domain of the disclosed CAR molecule. In some embodiments, the hinge-region may be rich in cysteine and proline amino acids In some embodiments, hinge regions useful in the present invention are generally derived from IgG subclasses (such as IgG1 and IgG4), IgD and CD8 domains, of which IgG1 has been most extensively used. In yet some further embodiments, hinge regions may be derived from the CD8a molecule or the CD28 molecule.

In yet some further embodiments, the second component of the disclosed CAR molecules is at least one transmembrane domain. A transmembrane domain (TMD) is a membrane-spanning domain that may traverse the membrane bilayer once or several times. TMDs may consist predominantly of nonpolar amino acid residues and generally adopt an alpha helix conformation. In some embodiments, the transmembrane domain comprises a natural transmembrane domain. In some embodiments, the transmembrane domain comprises an artificial transmembrane domain. In some embodiments, the transmembrane domain is an extension of a signal transduction domain also comprised within said CAR molecule.

In some embodiments, a natural transmembrane domain adjacent to a signal transduction domain polypeptide is used as the transmembrane domain for a CAR molecule. In some embodiments, a natural transmembrane domain adjacent to a signal transduction domain polypeptide is used as the transmembrane domain for a CAR cassette. In some embodiments, the natural transmembrane domain comprises the transmembrane domain selected from any of CD40 (TNR5), TLR4, ICOSL, CD80/CD86, IL2R, IL4R, UL5R, IL6R, IL10R, IL12R, IL13R, IL21R, TACI, BCMA, BAFF-R, or MHCII polypeptides. In some embodiments, a natural transmembrane domain of CD40 (TNR5), TLR4, ICOSL, CD80/CD86, IL2R, IL4R, UL5R, IL6R, IL10R, IL12R, IL13R, IL21R, TACI, BCMA, BAFF-R, or MHCII, is comprised within a CAR cassette.

In some embodiments, a transmembrane domain comprised within a CAR comprises the transmembrane domain of CD40 (TNR5). In some embodiments, a transmembrane domain comprised within a CAR comprises the transmembrane domain of TLR4. In some embodiments, a transmembrane domain comprised within a CAR comprises the transmembrane domain of ICOSL. In some embodiments, a transmembrane domain comprised within a CAR comprises the transmembrane domain of CD80/CD86. In some embodiments, a transmembrane domain comprised within a CAR comprises the transmembrane domain of IL2R. In some embodiments, a transmembrane domain comprised within a CAR comprises the transmembrane domain of IL4R. In some embodiments, a transmembrane domain comprised within a CAR comprises the transmembrane domain of UL5R. In some embodiments, a transmembrane domain comprised within a CAR comprises the transmembrane domain of IL6R. In some embodiments, a transmembrane domain comprised within a CAR comprises the transmembrane domain of IL10R. In some embodiments, a transmembrane domain comprised within a CAR comprises the transmembrane domain of IL12R. In some embodiments, a transmembrane domain comprised within a CAR comprises the transmembrane domain of IL13R. In some embodiments, a transmembrane domain comprised within a CAR comprises the transmembrane domain of IL21R. In some embodiments, a transmembrane domain comprised within a CAR comprises the transmembrane domain of TACI. In some embodiments, a transmembrane domain comprised within a CAR comprises the transmembrane domain of BCMA. In some embodiments, a transmembrane domain comprised within a CAR comprises the transmembrane domain of BAFF-R. In some embodiments, a transmembrane domain comprised within a CAR comprises the transmembrane domain of MHCII.

The transmembrane regions of CD40 and TLR4 are indicated in bold within SEQ ID NOs: 6 and 7, respectively, in the sequences provided following the Examples.

Still further, the third component of the CAR molecule of the present disclosure is at least one signal transduction domain. According to some embodiments, this domain is an intracellular domain connected to the transmembrane domain. It some embodiments, the signal transduction domain of the CAR of the present disclosure is at least one intracellular co-stimulatory domain that induces T cell independent B cell activation. In yet some further embodiments, this domain is not derived from a B cell receptor.

In some embodiments, the intracellular co-stimulatory domain is derived from any endogenously expressed receptor protein, naturally involved in co-stimulation of T-cell activation (proliferation and cytokine secretion).

Still further, in some embodiments, the intracellular co-stimulatory signal transduction domain comprises at least one of: (i) Major histocompatibility complex class II (MHCII), (ii) Cluster of differentiation 40 (CD40), (iii) Inducible co-stimulatory ligand (ICOSL), (iv) Cluster of differentiation 80/86 (CD80/CD86), (v) interleukin-2 receptor (IL2R), (vi) interleukin-4 receptor (IL4R), (vii) interleukin-5 receptor (IL5R), (viii) interleukin-6 receptor (IL6R), (ix) interleukin-10 receptor (IL10R), (x) interleukin-12 receptor (IL12R), (xi) interleukin-13 receptor (IL13R), (xii) interleukin-21 receptor (IL21R), (xiii) Transmembrane activator and CAML interactor (TACI), (xiv) B-cell maturation antigen (BCMA), (xv) B-cell activating factor receptor (BAFF-R), and any combinations thereof.

As discussed above, the CAR molecule of the present disclosure is characterized in that it allows a cell of the B cell lineage to undergo antigen-induced activation independent of a cell of the T cell lineage. In some embodiments, the cell of the B cell lineage is at least one of B cell precursor, naïve B cell and activated B cell of all phenotypes.

In certain embodiments, a CAR disclosed herein comprises a signal transduction domain comprising at least one intracellular co-stimulatory domain that induces T cell independent B cell activation, said intracellular co-stimulatory signal transduction domain comprising at least one of: (i) Cluster of differentiation 40 (CD40), (ii) Toll-like receptor 4 (TLR4), (iii) Inducible co-stimulatory ligand (ICOSL), (iv) Cluster of differentiation 80/86 (CD80/CD86), (v) interleukin-2 receptor (IL2R), (vi) interleukin-4 receptor (IL4R), (vii) interleukin-5 receptor (IL5R), (viii) interleukin-6 receptor (IL6R), (ix) interleukin-10 receptor (IL10R), (x) interleukin-12 receptor (IL12R), (xi) interleukin-13 receptor (IL13R), (xii) interleukin-21 receptor (IL21R), (xiii) Transmembrane activator and CAML interactor (TACI), (xiv) B-cell maturation antigen (BCMA), (xv) B-cell activating factor receptor (BAFF-R), (xvi) Major histocompatibility complex class II (MHCII), or any combinations thereof.

In some embodiments, the CAR molecule transduces antigen-induced B cell activation in the germinal centers allowing affinity maturation, class switch recombination and memory retention.

In yet some further embodiments, the antigen-induced activation of said cell of the B cell lineage results in generation of memory cells and long-lived antibody-secreting plasma cells.

In some particular and non-limiting embodiments, the CAR of the present disclosure may comprise an antigen binding domain of the Trastuzumab antibody and a TLR4 signaling domain. In some embodiments, the CAR molecule is therefore Trastuzumab CAR with TLR4 Signaling domain. Still further, in more specific embodiments, the CAR molecule may comprise the amino acid sequence as denoted by SEQ ID NO: 12, or any derivatives thereof. In yet some further alternative embodiments, the CAR of the present disclosure may comprise an antigen binding domain of the Trastuzumab antibody and a CD40 Signaling domain. In some embodiments, the CAR molecule is therefore Trastuzumab CAR with CD40 Signaling domain. Still further, in more specific embodiments, the CAR molecule may comprise the amino acid sequence as denoted by SEQ ID NO: 13, or any derivatives thereof.

In yet some further alternative embodiments, the CAR of the present disclosure may comprise an antigen binding domain of the 3BNC117 antibody and a TLR4 Signaling domain. In some embodiments, the CAR molecule is therefore 3BNC117 CAR with TLR4 Signaling domain. Still further, in more specific embodiments, the CAR molecule may comprise the amino acid sequence as denoted by SEQ ID NO: 14, or any derivatives thereof.

In yet some further alternative embodiments, the CAR of the present disclosure may comprise an antigen binding domain of the 3BNC117 antibody and a CD40 Signaling domain. In some embodiments, the CAR molecule is therefore 3BNC117 CAR with CD40 Signaling domain. Still further, in more specific embodiments, the CAR molecule may comprise the amino acid sequence as denoted by SEQ ID NO: 15, or any derivatives thereof.

In yet some further alternative embodiments, the CAR of the present disclosure may comprise an antigen binding domain of the Trastuzumab antibody and a CD40 and CD79b Signaling domain. In some embodiments, the CAR molecule is therefore Trastuzumab CAR with CD40 and CD79b Signaling domain. Still further, in more specific embodiments, the CAR molecule may comprise the amino acid sequence as denoted by SEQ ID NO: 16, or any derivatives thereof.

A further aspect of the present disclosure relates to a system comprising: (a) at least one CAR molecule; and (b) at least one effector protein; or (c) at least one nucleic acid molecule comprising at least one nucleic acid sequence encoding at least one of (a), (b) or (a) and (b), or any or any cassette, vector or vehicle comprising the at least one nucleic acid molecule. More specifically, the CAR molecule of the disclosed system allows a cell of the B cell lineage to undergo antigen-induced activation independent of a cell of the T cell lineage. In some embodiments, the CAR molecule of the disclosed system comprises the following components: (i) at least one target-binding domain; (ii) at least one transmembrane domain; and (iii) at least one signal transduction domain. It should be noted that the transduction domain of the disclosed CAR molecule is capable of relaying signals for T cell independent activation. More specifically, the transduction domain of the CAR molecule of the disclosed system is characterized in its ability to induce immune activation that is not dependent on T cells.

In certain embodiments, a system disclosed herein comprises:
(a) at least one CAR molecule that allows a cell of the B cell lineage to undergo antigen-induced activation independent of a cell of the T cell lineage as disclosed herein; and (b) at least one effector protein; or (c) at least one nucleic acid molecule comprising at least one nucleic acid sequence encoding at least one of (a), (b) or (a) and (b), or any or any cassette, vector or vehicle comprising the at least one nucleic acid molecule. More specifically, the CAR molecule of the disclosed system allows a cell of the B cell lineage to undergo antigen-induced activation independent of a cell of the T cell lineage. In some embodiments, the CAR molecule of the disclosed system comprises the following components: (i) at least one target-binding domain comprising at least one target-recognition element, wherein said target is at least one antigen associated with a pathologic disorder and said pathologic disorder is at least one of: a proliferative disorder, an inflammatory disorder, an infectious disease caused by a pathogen, an autoimmune disease, a neurodegenerative disease, a congenital disorder, an allergic condition, a cardiovascular disease and metabolic condition; (ii) at least one transmembrane domain; and (iii) at least one signal transduction domain, wherein said signal transduction domain is not derived from a B cell receptor and is capable of relaying signals for T cell independent activation; ad wherein said cell of the B cell lineage is at least one of B cell precursor, naïve B cell, and activated B cell of all phenotypes. It should be noted that the transduction domain of the disclosed CAR molecule is capable of relaying signals for T cell independent activation. More specifically, the transduction domain of the CAR molecule of the disclosed system is characterized in its ability to induce immune activation that is not dependent on T cells.

In yet some further embodiments, the system of the present disclosure comprises any of the CAR molecules as defined by the invention herein above.

In some embodiments of a CAR molecule, an at least one effector protein comprises (i) at least one membranal and/or a secreted antibody or antibody derivative; (ii) at least one Bi-specific T cell engager; (iii) at least one cytokine; (iv) at least one membranal receptor; and (v) at least one secreted toxin.

In some embodiments, an antibody derivative comprises a scFv, a BiTE, a single chain antibody, a bi-specific antibody, a camelid antibody, a nanobody, or an antigen binding fragment of an antibody.

In yet some further embodiments, in addition to the specific CAR molecules, the systems of the invention may comprise at least one effector protein. In more specific embodiments, such effector proteins may comprise at least one of: (i) at least one membranal and/or a secreted antibody or antibody derivative; (ii) at least one Bi-specific T cell engager; (iii) at least one cytokine; (iv) at least membranal receptor; and (v) at least one secreted toxin.

In some embodiments, the system of the present disclosure may comprise in addition to the CAR molecule, at least one membranal and/or secreted antibody. In some embodiments, such this antibody may recognize and binds the same or target (e.g., the same antigen or in some embodiments, even the same epitope). In yet some further embodiments, the at least one membranal and/or secreted antibody of the systems disclosed herein may recognize at least one different target molecule/s.

In some embodiments, the at least one target binding domain of the CAR molecule of the system of the present disclosure comprises: (i) at least one target-recognition element. In some additional or alternative embodiments, the at least one target binding domain may further, or alternatively comprise (ii) at least one adaptor component that recognizes and binds at least one tagged target-recognition element.

In yet some further embodiments, the target-recognition element of the CAR molecule of the system of the present disclosure comprises at least one antibody or any antigen-binding fragment/s, portion/s or chimera/s thereof.

In some embodiments, the adaptor component comprises at least one moiety that specifically recognizes and binds at least one tag of the tagged target-recognition element of the CAR molecule of the systems of the present disclosure. According to such specific embodiments, the CAR molecule of the present disclosure may be adapted for various target recognition elements that are tagged by a tag recognized and by the adaptor component, thereby forming a recognition pair. These CAR molecules may be also indicated herein as universal CARs. In some embodiments, the recognition pair may include the biotin/avidin affinity pair. Various affinity pairs are as disclosed herein above in connection with the CAR molecule aspect of the invention.

In some embodiments, the target targeted by the target binding domain of the CAR of the system of the present disclosure is at least one antigen associated with a pathologic disorder.

Still further, such target is an antigen associated with at least one pathologic disorder that may be in some embodiments, at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition.

In more specific embodiments of the disclosed systems, the proliferative disorder is cancer. Accordingly, the target-binding domain specifically recognizes and binds at least one target, specifically an antigen associated with, expressed by, or comprised within a tumor tissue (or tumor microenvironment, TME), or by at least one tumor cell. Thus, in some embodiments, the target is at least one tumor associated antigen (TAA).

In yet some further specific embodiments, the TAA recognized by the CAR molecule of the system of the present disclosure is the human epidermal growth factor receptor 2 (HER2/neu) or any fragment thereof. Thus, in some embodiments, the target binding domain of the CAR of the present disclosure specifically recognizes and binds HER2/neu.

In more specific embodiments, the target-recognition element of the CAR molecule of the system of the present disclosure, comprises at least one antibody or any antigen-binding fragment/s, portion/s or chimera/s thereof that specifically recognizes and binds HER2/neu.

Still further, in some non-limiting embodiments, an anti-HER2/neu antibody applicable in the present invention is the monoclonal antibody Trastuzumab, also known as Herceptin, Herzuma or Ogivri. Still further, in some embodiments, the disclosed CAR may comprise at least one of the variable heavy chain (VH) and/or the variable light chain (VL) of the Trastuzumab antibody. More specifically, in some embodiments the VH of Trastuzumab may comprise the amino acid sequence as denoted by SEQ ID NO: 8. Still further, in some embodiments the VL of Trastuzumab may comprise the amino acid sequence as denoted by SEQ ID NO:9.

In yet some further specific and non-limiting embodiments, the TAA is the human cytokine receptor gp130 or any fragment thereof. Thus, in some embodiments, the target binding domain of the CAR of the of the system of the present disclosure specifically recognizes and binds gp130.

In more specific embodiments, the target-recognition element of the disclosed CAR of the system of the present disclosure comprises at least one antibody or any antigen-binding fragment/s, portion/s or chimera/s thereof that specifically recognizes and binds gp130.

In yet some further specific and non-limiting embodiments, the TAA is the MAGE or any fragment thereof. Thus, in some embodiments, the target binding domain of the CAR of the systems of the present disclosure specifically recognizes and binds MAGE any other melanoma related antigens.

In more specific embodiments, the target-recognition element of the disclosed CAR of the system of the present disclosure comprises at least one antibody or any antigen-binding fragment/s, portion/s or chimera/s thereof that specifically recognizes and binds MAGE or any other melanoma related antigens.

In yet some alternative embodiments, the pathologic disorder is an infectious disease caused by a viral pathogen. Accordingly, the target-binding domain of the CAR molecule of the system of the present disclosure, specifically recognizes and binds at least one target, specifically an antigen associated with, expressed by, or comprised within a tissue and/or cell affected by a viral pathogen.

In some embodiments, the viral pathogen is the human immunodeficiency virus (HIV).

Still further, in some embodiments, wherein said target-recognition element comprises at least one antibody or any antigen-binding fragment/s, portion/s or chimera/s thereof that specifically recognizes and binds gp120. In some embodiments, the anti-gp120 antibodies are broadly neutralizing HIV-1 antibodies (bNAb). In more specific embodiments, the neutralizing HIV-1 antibody is 3BNC117. In yet some further embodiments, the VH of the 3BNC117 antibody may comprise the amino acid sequence as denoted by SEQ ID NO:10. Still further, in some embodiments the VL of 3BNC117 may comprise the amino acid sequence as denoted by SEQ ID NO:11.

Still further, in some embodiments, the target-recognition domain of the CAR molecule of the systems of the present disclosure is part of the extracellular domain of the molecule. In yet some further embodiments, the extracellular domain of the CAR molecule of the present disclosure my comprise at least one additional components or domains. More specifically, further components that may be added to the extracellular domain may comprise for example, at least one spacer domain comprising at least one tag or labeling moiety. Such labeling moiety may comprise for example, Strep-II, HA-tag and the like. In yet some further embodiments, the extracellular domain of the CAR molecule of the system of the present disclosure may comprise at least one hinge region.

In yet some further embodiments, the second component of the CAR molecules of the system of the present disclosure is at least one transmembrane domain.

Still further, the third component of the CAR molecule of the systems of the present disclosure is at least one signal transduction domain. According to some embodiments, this domain is an intracellular domain connected to the transmembrane domain. It some embodiments, the signal transduction domain of the CAR of the systems of the present disclosure is at least one intracellular co-stimulatory domain that induces T cell independent B cell activation. In yet some further embodiments, this domain is not derived from a B cell receptor.

In some embodiments, the intracellular co-stimulatory domain is derived from any endogenously expressed receptor protein, naturally involved in co-stimulation of T-cell activation (proliferation and cytokine secretion). Still further, in some embodiments, the intracellular co-stimulatory signal transduction domain comprises at least one of: (i) Major histocompatibility complex class II (MHCII), (ii) Cluster of differentiation 40 (CD40), (iii) Inducible co-stimulatory ligand (ICOSL), (iv) Cluster of differentiation 80/86 (CD80/CD86), (v) interleukin-2 receptor (IL2R), (vi) interleukin-4 receptor (IL4R), (vii) interleukin-5 receptor (IL5R), (viii) interleukin-6 receptor (IL6R), (ix) interleukin-10 receptor (IL10R), (x) interleukin-12 receptor (IL12R), (xi) interleukin-13 receptor (IL13R), (xii) interleukin-21 receptor (IL21R), (xiii) Transmembrane activator and CAML interactor (TACI), (xiv) B-cell maturation antigen (BCMA), (xv) B-cell activating factor receptor (BAFF-R), and any combinations thereof.

As discussed above, the CAR molecule of the systems of the present disclosure is characterized in that it allows a cell of the B cell lineage to undergo antigen-induced activation independent of a cell of the T cell lineage. In some embodiments, the cell of the B cell lineage is at least one of B cell precursor, naïve B cell and activated B cell of all phenotypes.

In some embodiments, the CAR molecule of the systems of the present disclosure transduces antigen-induced B cell activation in the germinal centers allowing affinity maturation, class switch recombination and memory retention.

In yet some further embodiments, the antigen-induced activation of said cell of the B cell lineage results in generation of memory cells and long-lived antibody-secreting plasma cells.

A further aspect of the present disclosure relates to a nucleic acid molecule comprising at least one nucleic acid sequence encoding at least one CAR that allows a cell of the B cell lineage to undergo antigen-induced activation independent of a cell of the T cell lineage, or any cassette, vector or vehicle comprising said nucleic acid molecule. In some embodiments the CAR encoded by the nucleic acid sequence of the present disclosure comprises the following components: (i) at least one target-binding domain; (ii) at least one transmembrane domain; and (iii) at least one signal transduction domain. It should be understood that this signal transduction domain is capable of relaying, and transducing signals for T cell independent activation.

In certain embodiments, disclosed herein is a nucleic acid molecule comprising at least one nucleic acid sequence encoding at least one CAR that allows a cell of the B cell lineage to undergo antigen-induced activation independent of a cell of the T cell lineage, or any cassette, vector or vehicle comprising said nucleic acid molecule, said nucleic acid sequence encoding said at least one CAR encoding 5′ to 3′ (i) a target-recognition element encoding an at least one antibody or any antigen-binding fragment/s, portion/s or chimera/s thereof that specifically recognizes an at least one antigen associated with a pathologic disorder, said pathologic disorder at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition; (ii) at least one transmembrane domain; and (iii) at least one signal transduction domain, wherein said signal transduction domain is not derived from a B cell receptor and is capable of relaying signals for T cell independent activation; wherein said nucleic acid molecule is optionally flanked on the 5′ and 3′ ends by homology arms for integration to a target site by homologous recombination; or is optionally flanked on at least one of the 5′ and 3′ ends thereof by at least one of: recognition sites for a site-specific nuclease, a site-specific integrase, or a site-specific recombinase.

In some embodiments, the disclosed nucleic acid molecule may further comprise at least one nucleic acid sequence encoding at least one effector protein. More specifically, the effector protein is at least one of: (i) at least one membranal and/or a secreted antibody or antibody derivative; (ii) at least one Bi-specific T cell engager; (iii) at least one cytokine; (iv) at least one membranal receptor; and (v) at least one secreted toxin. It should be understood that in some embodiments, the disclosed systems may comprise in addition to the CAR molecules of the invention, an effector protein that may be an engineered B cell receptor (BCR). In some embodiments, the present disclosure provides at least one nucleic acid molecule encoding any of the CAR molecules defined by the present disclosure.

In some embodiments, disclosed herein is a nucleic acid molecule comprising a nucleic acid sequence encoding a target recognition sequence that encodes 5′ to 3′a variable light chain region and a variable heavy chain region that specifically recognize at least one antigen associated with a proliferative disorder, wherein the disorder is cancer and the antigen isHER2, or wherein the variable light chain and variable heavy chain region specifically recognize at least one antigen associated with an infectious disease caused by a pathogen, wherein the pathogen is HIV and the antigen is gp120.

In some embodiments, the nucleic acid molecule further comprises a nucleic acid sequence upstream of the at least one nucleic acid sequence encoding said CAR and a nucleic acid sequence downstream of said at least one nucleic acid sequence encoding said CAR, wherein said upstream nucleic acid sequence encodes 5′ to 3′, a poly-adenylation site and an enhancer dependent promoter, and said downstream nucleic acid sequence encodes 5′ to 3′, at least one membranal and/or secreted antibody or antibody derivative, and a splice donor site. In some embodiments of a nucleic acid molecule, said at least one membranal and/or secreted antibody or antibody derivative specifically recognizes and binds HER2 or gp120.

Still further, in some embodiments, the nucleic acid molecule encoding the CAR molecule of the present disclosure and/or the effector protein, is flanked on at least one of the 5′ and 3′ thereof by at least one of: (i) homology arms, for integration to a target site by homologous recombination; and (ii) recognition sites for a site-specific nuclease, a site-specific integrase or a site-specific recombinase.

In some embodiments, the nucleic acid molecule encoding a CAR molecule, as disclosed herein, wherein said nucleic acid molecule is flanked on the 5′ and 3′ ends by homology arms for integration to a target site by homologous recombination; or is optionally flanked on at least one of the 5′ and 3′ ends thereof by at least one of: recognition sites for a site-specific nuclease, a site-specific integrase, or a site-specific recombinase.

In yet some further embodiments, the nucleic acid sequences of the present invention may be comprised within at least one nucleic acid cassette. Still further, in some embodiments, the nucleic acid molecules of the present disclosure or any nucleic acid cassettes thereof may be comprised within t least one vector. In some embodiments of the present disclosure, a vector suitable in the present disclosure is any one of a viral vector, a non-viral vector and a naked DNA vector.

A further aspect of the present disclosure relates to a gene editing system comprising: (i) at least one nucleic acids molecule as disclosed herein throughout, or any cassette, vector or vehicle comprising said at least one nucleic acid molecule; and (ii) at least one gene editing component or a nucleic acid sequence encoding the gene editing component.

In some embodiments, a gene editing component may be any one of a site-specific nuclease, a class switch recombination, a site specific integrase, a site-specific recombinase and a recombination activating gene (RAG)-catalyzed recombination.

In some specific embodiments, a gene editing component useful in the systems of the present disclosure may be the CRISPR/Cas.

In some embodiments, the present disclosure further encompasses at least one host cell comprising and/or expressing the nucleic acid molecule of the present disclosure or any system comprising the same.

A further aspect of the present disclosure relates to a genetically engineered cell of the B cell lineage expressing at least one CAR molecule, or any population of cells comprising at least one of the genetically modified cell/s of the present disclosure. The engineered cell of the present disclosure is capable of undergoing antigen-induced activation independent of a cell of the T cell lineage. In yet some further embodiments, the CAR molecule of the engineered cell/s of the present disclosure comprises: (i) at least one target-binding domain; (ii) at least one transmembrane domain; and (iii) at least one signal transduction domain. It should be noted that the signal transduction domain of the CAR of the engineered cell/s of the present disclosure is capable of relaying signals for T cell independent activation.

In some embodiments, the genetically engineered cell of the present disclosure comprises and expresses any of the CAR molecules as defined by the present disclosure herein above.

In certain embodiments, disclosed herein is a genetically engineered cell of the B cell lineage expressing at least one CAR molecule, or any population of cells comprising at least one said genetically modified cell of the B cell lineage, wherein said cell is capable of undergoing antigen-induced activation independent of a cell of the T cell lineage, and wherein said CAR comprises:
(i) at least one target-binding domain comprising at least one target-recognition element, wherein said target is at least one antigen associated with a pathologic disorder and said pathologic disorder is at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition;
(ii) at least one transmembrane domain; and
(iii) at least one signal transduction domain, wherein said signal transduction domain is not derived from a B cell receptor and is capable of relaying signals for T cell independent activation; and
   wherein said cell of the B cell lineage is at least one of B cell precursor, naïve B cell, and activated B cell of all phenotypes.

In some embodiments, the engineered cell/s of the present disclosure further express at least one effector protein. More specifically, such effector protein is at least one of: (i) at least one membranal and/or a secreted antibody or antibody derivative; (ii) at least one Bi-specific T cell engager; (iii) at least one cytokine; (iv) at least membranal receptor; and (v) at least one secreted toxin. In some embodiments, the engineered cell of the present disclosure may comprise at least one engineered BCR.

In some embodiments, the genetically engineered cell disclosed herein is engineered in vivo in a subject in need. In yet some alternative embodiments, the engineering of the CAR of the cell is conducted ex vivo.

In some embodiments, the at least one target binding domain of the CAR molecule of the engineered cell of the present disclosure comprises: (i) at least one target-recognition element. In some additional or alternative embodiments, the at least one target binding domain may further, or alternatively comprise (ii) at least one adaptor component that recognizes and binds at least one tagged target-recognition element.

In yet some further embodiments, the target-recognition element of the CAR molecule of the engineered cell of the present disclosure comprises at least one antibody or any antigen-binding fragment/s, portion/s or chimera/s thereof.

It should be understood that in some embodiments, the binding moieties of the target-recognition element of the CAR molecule of the engineered cell of the present disclosure may comprise a single antigen specificity (monospecific), or alternatively, two, three or more antigen specificities. Thus, in certain embodiments, the target binding moiety of the CAR molecule of the engineered cell of the present disclosure, may comprise antibodies or antigen binding fragment thereof that are directed to two o more antigens (e.g., bi-specific, or tri-specific antibodies).

In some embodiments, the adaptor component comprises at least one moiety that specifically recognizes and binds at least one tag of said tagged target-recognition element. According to such specific embodiments, the CAR molecule of the engineered cell of the present disclosure may be adapted for various target recognition elements that are tagged by a tag recognized and by the adaptor component, thereby forming a recognition pair. These CAR molecules may be also indicated herein as universal CARs. In some embodiments, the recognition pair may include the biotin/avidin affinity pair, or any of the affinity pairs disclosed by the present disclosure.

In some embodiments, the target targeted by the target binding domain of the CAR of the engineered cell of the present disclosure is at least one antigen associated with a pathologic disorder.

Still further, such target is an antigen associated with at least one pathologic disorder that may be in some embodiments, at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition.

In more specific embodiments, the proliferative disorder is cancer. Accordingly, the target-binding domain specifically recognizes and binds at least one target, specifically an antigen associated with, expressed by or comprised within a tumor tissue (or tumor microenvironment, TME), or by at least one tumor cell. Thus, in some embodiments, the target is at least one tumor associated antigen (TAA).

In yet some further specific embodiments, the TAA is the human epidermal growth factor receptor 2 (HER2/neu) or any fragment thereof. Thus, in some embodiments, the target binding domain of the CAR of the engineered cell of the present disclosure specifically recognizes and binds HER2/neu.

In more specific embodiments, the target-recognition element of the CAR of the engineered cell of the present disclosure comprises at least one antibody or any antigen-binding fragment/s, portion/s or chimera/s thereof that specifically recognizes and binds HER2/neu.

Still further, in some non-limiting embodiments, an anti-HER2/neu antibody applicable in the present invention is the monoclonal antibody Trastuzumab, also known as Herceptin, Herzuma or Ogivri. Still further, in some embodiments, the disclosed CAR may comprise at least one of the variable heavy chain (VH) and/or the variable light chain (VL) of the Trastuzumab antibody. More specifically, in some embodiments the VH of Trastuzumab may comprise the amino acid sequence as denoted by SEQ ID NO: 8. Still further, in some embodiments the VL of Trastuzumab may comprise the amino acid sequence as denoted by SEQ ID NO:9.

In yet some alternative embodiments, the pathologic disorder is an infectious disease caused by a viral pathogen. Accordingly, the target-binding domain of the CAR molecule of the engineered cell of the present disclosure, specifically recognizes and binds at least one target, specifically an antigen associated with, expressed by or comprised within a tissue and/or cell affected by a viral pathogen.

In some embodiments, the viral pathogen is the human immunodeficiency virus (HIV).

Still further, in some embodiments, wherein said target-recognition element comprises at least one antibody or any antigen-binding fragment/s, portion/s or chimera/s thereof that specifically recognizes and binds gp120. In some embodiments, the anti-gp120 antibodies are broadly neutralizing HIV-1 antibodies (bNAb). In more specific embodiments, the neutralizing HIV-1 antibody is 3BNC117. In yet some further embodiments, the VH of the 3BNC117 antibody may comprise the amino acid sequence as denoted by SEQ ID NO: 10. Still further, in some embodiments the VL of 3BNC117 may comprise the amino acid sequence as denoted by SEQ ID NO: 11.

In certain embodiments, in a genetically engineered cell disclosed herein, when the target is associated with a proliferative disorder and the proliferative disorder is cancer, the target-binding domain specifically recognizes at least one tumor associated antigen (TAA), wherein optionally, said TAA is the human epidermal growth factor receptor 2 (HER2/neu) or any fragment thereof and said target-recognition element comprises at least one antibody or any antigen-binding fragment/s, portion/s or chimera/s thereof that specifically recognizes and binds HER2/neu; or when the target is associated with an infectious disease caused by a pathogen and the pathogen is the human immunodeficiency virus (HIV), the target-recognition element comprises at least one antibody or any antigen-binding fragment/s, portion/s or chimera/s thereof that specifically recognizes and binds gp120.

Still further, in some embodiments, the target-recognition domain of the CAR molecule of the present disclosure is part of the extracellular domain of the molecule. In yet some further embodiments, the extracellular domain of the CAR molecule of the engineered cell of the present disclosure may comprise at least one additional components or domains. More specifically, further components that may be added to the extracellular domain may comprise for example, at least one spacer domain comprising at least one tag or labeling moiety. Such labeling moiety may comprise for example, Strep-II, HA-tag and the like. In yet some further embodiments, the extracellular domain of the CAR molecule of the engineered cell of the present disclosure may comprise at least one hinge region. The hinge region, as used herein, is a flexible amino acid stretch/structure, which may in some embodiments link between the target-binding domain and the transmembrane domain of the CAR molecule of the engineered cell of the present disclosure.

In yet some further embodiments, the second component of the disclosed CAR molecules is at least one transmembrane domain.

Still further, the third component of the CAR molecule of the engineered cell of the present disclosure is at least one signal transduction domain. According to some embodiments, this domain is an intracellular domain connected to the transmembrane domain. It some embodiments, the signal transduction domain of the CAR of the engineered cell of the present disclosure is at least one intracellular co-stimulatory domain that induces T cell independent B cell activation. In yet some further embodiments, this domain is not derived from a B cell receptor.

In some embodiments, the intracellular co-stimulatory domain is derived from any endogenously expressed receptor protein, naturally involved in co-stimulation of T-cell activation (proliferation and cytokine secretion).

Still further, in some embodiments, the intracellular co-stimulatory signal transduction domain comprises at least one of: (i) Major histocompatibility complex class II (MHCII), (ii) Cluster of differentiation 40 (CD40), (iii) Inducible co-stimulatory ligand (ICOSL), (iv) Cluster of differentiation 80/86 (CD80/CD86), (v) interleukin-2 receptor (IL2R), (vi) interleukin-4 receptor (IL4R), (vii) interleukin-5 receptor (IL5R), (viii) interleukin-6 receptor (IL6R), (ix) interleukin-10 receptor (IL10R), (x) interleukin-12 receptor (IL12R), (xi) interleukin-13 receptor (IL13R), (xii) interleukin-21 receptor (IL21R), (xiii) Transmembrane activator and CAML interactor (TACI), (xiv) B-cell maturation antigen (BCMA), (xv) B-cell activating factor receptor (BAFF-R), and any combinations thereof. In some embodiments, a signal transduction domain comprises at least one intracellular co-stimulatory domain that induces T cell independent B cell activation, and said intracellular co-stimulatory signal transduction domain comprises at least one of: (i) Cluster of differentiation 40 (CD40), (ii) Toll-like receptor 4 (TLR4), (iii) Inducible co-stimulatory ligand (ICOSL), (iv) Cluster of differentiation 80/86 (CD80/CD86), (v) interleukin-2 receptor (IL2R), (vi) interleukin-4 receptor (IL4R), (vii) interleukin-5 receptor (IL5R), (viii) interleukin-6 receptor (IL6R), (ix) interleukin-10 receptor (IL10R), (x) interleukin-12 receptor (IL12R), (xi) interleukin-13 receptor (IL13R), (xii) interleukin-21 receptor (IL21R), (xiii) Transmembrane activator and CAML interactor (TACI), (xiv) B-cell maturation antigen (BCMA), (xv) B-cell activating factor receptor (BAFF-R), Major histocompatibility complex class II (MHCII), and any combinations thereof.

As discussed above, the CAR molecule of the engineered cell of the present disclosure is characterized in that it allows a cell of the B cell lineage to undergo antigen-induced activation independent of a cell of the T cell lineage. In some embodiments, the cell of the B cell lineage is at least one of B cell precursor, naïve B cell and activated B cell of all phenotypes.

In some embodiment of a genetically engineered cell, the cell further expresses and or secretes at least one effector protein, said effector protein is an at least one membranal and/or secreted antibody or antibody derivative. In some embodiment of a genetically engineered cell, the cell further expresses and or secretes at least one effector protein, said effector protein is (i) an at least one membranal and/or secreted antibody or antibody derivative; (ii) at least one Bi-specific T cell engager; (iii) at least one cytokine; (iv) at least membranal receptor; and (v) at least one secreted toxin.

In some embodiments, the CAR molecule transduces antigen-induced B cell activation in the germinal centers allowing affinity maturation, class switch recombination and memory retention.

In yet some further embodiments, the antigen-induced activation of said cell of the B cell lineage results in generation of memory cells and long-lived antibody-secreting plasma cells.

A further aspect of the present disclosure relates to a pharmaceutical composition comprising at least one CAR molecule that allows a cell of the B cell lineage to undergo antigen-induced activation independent of a cell of the T cell lineage, or any system comprising the CAR molecule disclosed herein, any nucleic acid molecule comprising at least one nucleic acid sequence encoding the CAR molecule, or any, cassette, vector, vehicle or gene editing system comprising the nucleic acid molecule, any cell expressing the CAR molecule of the present disclosure, or any genetically engineered B cell expressing the CAR molecule or population of cells comprising at least one the genetically engineered B cell disclosed herein. In some embodiments, the CAR molecule comprises the following components: (i) at least one target-binding domain; (ii) at least one transmembrane domain; and (iii) at least one signal transduction domain. It should be understood that this signal transduction domain is capable of relaying, and transducing signals for T cell independent activation. In some optional embodiments, the composition disclosed herein may further comprise at least one of pharmaceutically acceptable carrier/s, diluent/s, excipient/s and additive/s.

In some embodiments, the pharmaceutical composition of the present disclosure may comprise any of the CAR molecules as defined by the invention, an of the system/s disclosed herein, any of the nucleic acid molecules as defined by the present disclosure, any gene editing system defined by the present disclosure, and any of the engineered cells as defined by the present disclosure.

In certain embodiments, disclosed herein is a pharmaceutical composition comprising
at least one CAR that allows a cell of the B cell lineage to undergo antigen-induced activation independent of a cell of the T cell lineage, or any nucleic acid molecule comprising at least one nucleic acid sequence encoding said CAR, or any, cassette, vector, vehicle, or gene editing system comprising said nucleic acid molecule, any cell expressing said CAR, or any genetically engineered B cell expressing said CAR or population of cells comprising at least one said genetically engineered B cell, wherein said nucleic acid sequence encoding said at least one CAR encodes 5′ to 3′ (i) a target-recognition element encoding an at least one antibody or any antigen-binding fragment/s, portion/s or chimera/s thereof that specifically recognizes an at least one antigen associated with a pathologic disorder, said pathologic disorder at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition; (ii) at least one transmembrane domain; and (iii) at least one signal transduction domain, wherein said signal transduction domain is not derived from a B cell receptor and is capable of relaying signals for T cell independent activation; wherein said nucleic acid molecule is optionally flanked on the 5′ and 3′ ends by homology arms for integration to a target site by homologous recombination; or is optionally flanked on at least one of the 5′ and 3′ ends thereof by at least one of: recognition sites for a site-specific nuclease, a site-specific integrase, or a site-specific recombinase; and
at least one pharmaceutically acceptable carrier/s, diluent/s, excipient/s, or additive/s, or a combination thereof.

A further aspect of the resent disclosure relates to a method for genetic engineering of a cell of the B cell lineage to express at least one CAR molecule that allows the cell to undergo antigen-induced activation independent of a cell of the T cell lineage. The method comprising the step of contacting the cell of the B lineage with an effective amount of at least one nucleic acid molecule comprising at least one nucleic acid sequence encoding said CAR molecule or a system comprising the CAR, or any cassette, vehicle, vector or gene editing system comprising the nucleic acid molecule, or with a composition thereof. In some embodiments, the CAR of the disclosed methods comprises the following components: (i) at least one target-binding domain; (ii) at least one transmembrane domain; and (iii) at least one signal transduction domain. It should be understood that this signal transduction domain is capable of relaying, and transducing signals for T cell independent activation.

In some embodiments of the disclosed methods, the cell is engineered to further express at least one effector molecule or any system thereof, the effector protein further expressed by the cell is at least one of: (i) at least one membranal and/or a secreted antibody or antibody derivative; (ii) at least one Bi-specific T cell engager; (iii) at least one cytokine; (iv) at least membranal receptor; and (v) at least one secreted toxin. In certain embodiments, disclosed herein is a method for genetic engineering of a cell of the B cell lineage to express at least one CAR molecule that allows said cell to undergo antigen-induced activation independent of a cell of the T cell lineage, the method comprising the step of contacting said cell with an effective amount of at least one nucleic acid molecule comprising at least one nucleic acid sequence encoding said CAR or a system comprising said CAR, or any cassette, vehicle, vector, or gene editing system comprising said nucleic acid molecule, or with a composition thereof, wherein said nucleic acid sequence encoding CAR encodes 5′ to 3′ (i) a target-recognition element encoding an at least one antibody or any antigen-binding fragment/s, portion/s or chimera/s thereof that specifically recognizes an at least one antigen associated with a pathologic disorder, said pathologic disorder at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition; (ii) at least one transmembrane domain; and (iii) at least one signal transduction domain, wherein said signal transduction domain is not derived from a B cell receptor and is capable of relaying signals for T cell independent activation; wherein said nucleic acid molecule is optionally flanked on the 5′ and 3′ ends by homology arms for integration to a target site by homologous recombination; or is optionally flanked on at least one of the 5′ and 3′ ends thereof by at least one of: recognition sites for a site-specific nuclease, a site-specific integrase, or a site-specific recombinase; and
wherein said cell of the B cell lineage is at least one of B cell precursor, naïve B cell, and activated B cell of all phenotypes.

In some embodiments of the disclosed methods, the CAR molecule is as defined by the present disclosure, the system is as defined by the present disclosure, the nucleic acid molecule is as defined by the present disclosure and the gene editing system is as defined by the present disclosure.

In some embodiments, the method of the present disclosure is for genetic engineering of a B cell in a mammalian subject. Thus, in some embodiments, the cell of the B cell linage is contacted with at least one nucleic acid sequence encoding the CAR molecule of the invention, in a subject. Therefore, the contacting step may comprise in some embodiments, the administering to the subject the effective amount of the at least one nucleic acid molecule comprising at least one nucleic acid sequence encoding said CAR molecule or a system comprising the CAR, or any cassette, vehicle, vector or gene editing system comprising the nucleic acid molecule, or with a composition thereof.

In some embodiments of a method for genetic engineering of a cell of the B cell lineage, the cell is engineered to further express at least one effector molecule, wherein said effector protein is an at least one membranal and/or a secreted antibody or antibody derivative. In some embodiments of a method for genetic engineering of a cell of the B cell lineage, the cell is engineered to further express at least one effector molecule, wherein said effector protein is (i) an at least one membranal and/or secreted antibody or antibody derivative; (ii) at least one Bi-specific T cell engager; (iii) at least one cytokine; (iv) at least membranal receptor; and (v) at least one secreted toxin.

The present disclosure further provides a cell of the B cell lineage prepared by the method as disclosed by the present disclosure.

A further aspect of the present disclosure relates to a method for genetic engineering of a cell of the B cell lineage to express at least one CAR molecule that allows a cell of the B cell lineage to undergo antigen-induced activation independent of a cell of the T cell lineage in a subject in need thereof. In some embodiments, the disclosed method comprises the step of administering to the subject an effective amount of at least one nucleic acid molecule comprising at least one nucleic acid sequence encoding the CAR molecule or a system comprising the CAR, or any cassette, vehicle, vector or gene editing system comprising the nucleic acid molecule, or a composition thereof. In some embodiments, the CAR of the disclosed methods comprises the following components: (i) at least one target-binding domain; (ii) at least one transmembrane domain; and (iii) at least one signal transduction domain. It should be understood that this signal transduction domain is capable of relaying, and transducing signals for T cell independent activation.

In some embodiments of a method for genetic engineering of a cell of the B cell lineage, said contacting the cell comprises administering to the subject an effective amount of at least one at least one nucleic acid molecule comprising at least one nucleic acid sequence encoding said CAR or a system comprising said CAR, or any cassette, vehicle, vector, or gene editing system comprising said nucleic acid molecule, or with a composition thereof, wherein said nucleic acid sequence encoding CAR encodes 5′ to 3′ (i) a target-recognition element encoding an at least one antibody or any antigen-binding fragment/s, portion/s or chimera/s thereof that specifically recognizes an at least one antigen associated with a pathologic disorder, said pathologic disorder at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition; (ii) at least one transmembrane domain; and (iii) at least one signal transduction domain, wherein said signal transduction domain is not derived from a B cell receptor and is capable of relaying signals for T cell independent activation; wherein said nucleic acid molecule is optionally flanked on the 5′ and 3′ ends by homology arms for integration to a target site by homologous recombination; or is optionally flanked on at least one of the 5′ and 3′ ends thereof by at least one of: recognition sites for a site-specific nuclease, a site-specific integrase, or a site-specific recombinase.

In some embodiments of the disclosed methods, the CAR molecule is as defined by the present disclosure, the system is as defined by the present disclosure, the nucleic acid molecule is as defined by the present disclosure, and the gene editing system is as defined by the present disclosure.

A further aspect of the present disclosure relates to an effective amount of at least one nucleic acid molecule comprising at least one nucleic acid sequence encoding at least one CAR or a system comprising said CAR, or any cassette, vehicle, vector or gene editing system comprising the nucleic acid molecule, or a composition thereof for use in a method for genetic engineering of a cell of the B cell lineage to express said CAR molecule in a subject in need thereof. In some embodiments, the CAR molecule comprises the following components: (i) at least one target-binding domain; (ii) at least one transmembrane domain; and (iii) at least one signal transduction domain. It should be understood that this signal transduction domain is capable of relaying, and transducing signals for T cell independent activation.

In some embodiments of the at least one nucleic acid molecule for use according to the present disclosure, the CAR molecule is as defined by the present disclosure, the system is as defined by the present disclosure, the nucleic acid molecule is as defined by the present disclosure, and the gene editing system is as defined by the present disclosure.

A further aspect of the present disclosure relates to a method for treating, preventing, ameliorating, inhibiting or delaying the onset of a pathologic disorder in a mammalian subject, said method comprising the step of administering to said subject an effective amount of at least one of: (a) at least one nucleic acid molecule encoding least one CAR molecule that allows a cell of the B cell lineage to undergo antigen-induced activation independent of a cell of the T cell, or at least one system comprising said CAR; (b) at least one cassette, vector vehicle or gene editing system comprising said nucleic acid molecule of (a); (c) at least one cell expressing said CAR or a system comprising said CAR, or a population of said cells; and (d) a composition comprising at least one of (a), (b) and (c). In some embodiments, the CAR molecule used by the methods of the present disclosure comprises the following components: (i) at least one target-binding domain; (ii) at least one transmembrane domain; and (iii) at least one signal transduction domain. It should be understood that this signal transduction domain is capable of relaying, and transducing signals for T cell independent activation.

In some embodiments of the disclosed methods, the CAR molecule is as defined by the present disclosure, the system is as defined by the present disclosure, the nucleic acid molecule is as defined by the present disclosure, and the gene editing system is as defined by the present disclosure, the cell is as defined by the present disclosure and the composition is as defined by the present disclosure.

In certain embodiments, disclosed herein is a method for treating, preventing, ameliorating, inhibiting or delaying the onset of a pathologic disorder in a mammalian subject, said method comprising the step of administering to said subject an effective amount of at least one of:
(a) at least one nucleic acid molecule encoding at least one CAR that allows a cell of the B cell lineage to undergo antigen-induced activation independent of a cell of the T cell lineage or encoding at least one system comprising said CAR;
(b) at least one cassette, vector vehicle, or gene editing system comprising said nucleic acid molecule of (a);
(c) at least one cell expressing said CAR or a system comprising said CAR, or a population of said cells; or
(d) a composition comprising at least one of (a), (b) and (c);
wherein said CAR comprises:
   at least one target-binding domain comprising at least one target-recognition element, wherein said target is at least one antigen associated with a pathologic disorder and said pathologic disorder is at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition;
   at least one transmembrane domain; and
   at least one signal transduction domain, wherein said signal transduction domain is not derived from a B cell receptor and is capable of relaying signals for T cell independent activation; and
wherein said cell of the B cell lineage is at least one of B cell precursor, naïve B cell, and activated B cell of all phenotypes.

In some embodiments of the therapeutic methods disclosed herein, the at least one target binding domain of said CAR molecule comprises: (i) at least one target-recognition element; and/or (ii) at least one adaptor component that recognizes and binds at least one tagged target-recognition element.

In some embodiments, the target-recognition element of the CAR of the disclosed methods comprises at least one of: at least one antibody or any antigen-binding fragment/s, portion/s or chimera/s thereof.

In some embodiments of the disclosed methods, the adaptor component of the CAR molecules, comprises at least one moiety that specifically recognizes and binds at least one tag of the tagged target-recognition element.

In some embodiments of the disclosed methods, the target recognized by the target binding domain of the CAR, is at least one antigen associated with the pathologic disorder. In some embodiments, the antigen recognized by the CAR molecule used herein is the same pathologic disorder that affects the treated subject. Thus, in some embodiments, the target is an antigen associated with at least one pathologic disorder that may be in some embodiments, at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition.

In some embodiments, the proliferative disorder is cancer. Accordingly, the target-binding domain specifically recognizes and binds at least one target, specifically an antigen associated with, expressed by or comprised within a tumor tissue (or tumor microenvironment, TME), or by at least one tumor cell. Thus, in some embodiments, the target is at least one TAA.

In yet some further embodiments, the TAA is the human epidermal growth factor receptor 2 (HER2/neu) or any fragment thereof.

Still further, in some embodiments, target-recognition element of the CAR comprises at least one antibody or any antigen-binding fragment/s, portion/s or chimera/s thereof that specifically recognizes and binds HER2/neu. In more specific embodiments, the target-recognition element of the disclosed CAR comprises at least one antibody or any antigen-binding fragment/s, portion/s or chimera/s thereof that specifically recognizes and binds HER2/neu.

Still further, in some non-limiting embodiments, an anti-HER2/neu antibody applicable in the present invention is the monoclonal antibody Trastuzumab, also known as Herceptin, Herzuma or Ogivri. Still further, in some embodiments, the disclosed CAR may comprise at least one of the variable heavy chain (VH) and/or the variable light chain (VL) of the Trastuzumab antibody. More specifically, in some embodiments the VH of Trastuzumab may comprise the amino acid sequence as denoted by SEQ ID NO:8. Still further, in some embodiments the VL of Trastuzumab may comprise the amino acid sequence as denoted by SEQ ID NO:9.

In yet some further embodiments, the TAA is the gp130 or any fragment thereof.

Still further, in some embodiments, target-recognition element of the CAR comprises at least one antibody or any antigen-binding fragment/s, portion/s or chimera/s thereof that specifically recognizes and binds gp130. In more specific embodiments, the target-recognition element of the disclosed CAR comprises at least one antibody or any antigen-binding fragment/s, portion/s or chimera/s thereof that specifically recognizes and binds gp130.

In yet some further embodiments, the TAA is the MAGE or any fragment thereof.

Still further, in some embodiments, target-recognition element of the CAR comprises at least one antibody or any antigen-binding fragment/s, portion/s or chimera/s thereof that specifically recognizes and binds MAGE. In more specific embodiments, the target-recognition element of the disclosed CAR comprises at least one antibody or any antigen-binding fragment/s, portion/s or chimera/s thereof that specifically recognizes and binds MAGE.

In yet some alternative embodiments of the disclosed methods, the pathologic disorder is an infectious disease caused by a viral pathogen. Accordingly, the target-binding domain of the disclosed CAR molecule, specifically recognizes and binds at least one target, specifically an antigen associated with, expressed by or comprised within a tissue and/or cell affected by a viral pathogen.

In some embodiments of a method of treating herein, the viral pathogen is the human immunodeficiency virus (HIV).

Still further, in some embodiments of the disclosed methods, target-recognition element comprises at least one antibody or any antigen-binding fragment/s, portion/s or chimera/s thereof that specifically recognizes and binds gp120. In some embodiments, the anti-gp120 antibodies are broadly neutralizing HIV-1 antibodies (bNAb). In more specific embodiments, the neutralizing HIV-1 antibody is 3BNC117. In yet some further embodiments, the VH of the 3BNC117 antibody may comprise the amino acid sequence as denoted by SEQ ID NO: 10. Still further, in some embodiments the VL of 3BNC117 may comprise the amino acid sequence as denoted by SEQ ID NO:11.

In some embodiments of a method of treating disclosed herein, the proliferative disorder is cancer, and the target-binding domain of said CAR specifically recognizes at least one tumor associated antigen (TAA), and wherein optionally said TAA is the human epidermal growth factor receptor 2 (HER2/neu) or any fragment thereof and said target-recognition element of said CAR comprises at least one antibody or any antigen-binding fragment/s, portion/s or chimera/s thereof that specifically recognizes and binds HER2/neu.

In other embodiments of methods of treating disclosed herein, wherein the disorder is an infectious disease caused by a pathogen, the pathogen is the human immunodeficiency virus (HIV) and the target-recognition element of said CAR comprises at least one antibody or any antigen-binding fragment/s, portion/s or chimera/s thereof that specifically recognizes and binds gp120.

In yet some further embodiments of the disclosed methods, the signal transduction domain of the CAR molecule is at least one intracellular co-stimulatory domain that induces T cell independent B cell activation, said domain is not derived from a B cell receptor, optionally, said intracellular co-stimulatory signal transduction domain comprises at least one of: (i) MHCII, (ii) CD40, (iii) ICOSL, (iv) CD80/CD86, (v) IL2R, (vi) IL4R, (vii) IL5R, (viii) IL6R, (ix) IL10R, (x) IL12R, (xi) IL13R, (xii) IL21R, (xiii) TACI, (xiv) BCMA, (xv) BAFF-R, or any combinations thereof.

In some embodiments, the subject is administered with the at least one nucleic acid molecule comprising at least one nucleic acid sequence encoding said CAR or a system comprising the CAR, or any cassette, vector or gene editing system comprising the nucleic acid molecule. In some embodiments, the nucleic acid molecule is flanked on at least one of the 5′ and 3′ thereof by at least one of (i) homology arms, for integration by homologous recombination; and (ii) recognition sites for a site-specific nuclease, a site-specific integrase or a site-specific recombinase.

In some embodiments, the editing of at least one B cell in the treated subject to express said CAR (insertion of the nucleic acid sequence encoding the CAR into the target genomic locus), is mediated by at least one of a site-specific nuclease, a class switch recombination, a site specific integrase, a site-specific recombinase and a recombination activating gene (RAG)-catalyzed recombination.

In some embodiments, the insertion is mediated by a site-specific nuclease. Optionally, the nuclease is at least one programmable engineered nuclease (PEN). In some embodiments, the PEN comprises at least one clustered regulatory interspaced short palindromic repeat (CRISPR)/CRISPR associated (cas) protein system. Accordingly, the method comprises administering to the subject at least one of: (I) at least one nucleic acid molecule comprising at least one nucleic acid sequence encoding said CAR molecule or a system comprising said CAR, or any system, cassette, vehicle or vector thereof; and (II) at least one of: (a) at least one CRISPR/cas protein, or any nucleic acid molecule encoding said Cas protein; and (b) at least one nucleic acid sequence comprising at least one guide RNA (gRNA) that targets the insertion of the nucleic acid molecule comprising at least one nucleic acid sequence encoding said CAR or a system comprising the CAR, into a target genomic sequence, or any nucleic acid sequence encoding said gRNA; or (c) any kit, composition or vehicle comprising at least one of (a) and (b); or (III) any gene editing system comprising (I), (II), or (I) and (II). In some embodiments, the therapeutic methods of the present disclosure encompass an in vivo gene editing of the cells of the B linage in the treated subject.

In yet some further embodiments, the insertion is mediated by a class witch recombination catalyzed by activation induced cytidine deaminase (AID).

In some embodiments, the present disclosure provides ex vivo methods. More specifically, in some embodiments, the subject is administered with at least one cell expressing the CAR, or a population of said cells. According to more specific embodiments, the cells are of an autologous or allogeneic source.

Still further, in some embodiments of the methods of the present disclosure are applicable for the treatment of any pathologic disorder disclosed by the present disclosure. More specifically, at least one of a proliferative disorder, an inflammatory disorder, an infectious disease caused by a pathogen, an autoimmune disease, a neurodegenerative disease, a congenital disorder, an allergic condition, a cardiovascular disease, and a metabolic condition.

In some embodiments, the therapeutic methods of the present invention are particularly applicable in mammalian subject that suffers from immune tolerance to a self-antigen involved in the condition, suffers from a medical condition compromising its immunity, receives immune-suppressive or immune-ablating treatments, and/or suffers from medical conditions, such as cancer that may involve active immunosuppression and immune exclusion.

In yet some further embodiments, it should be understood that the CAR molecules used in the disclosed methods and any cell of the B lineage expressing these CAR molecules are directed against a target antigen associated with, expressed by, related to the pathologic disorder of the treated subject.

The present disclosure further provides an effective amount of at least one of: (a) at least one nucleic acid molecule encoding least one CAR that allows a cell of the B cell lineage to undergo antigen-induced activation independent of a cell of the T cell, or at least one system comprising said CAR; (b) at least one cassette, vector vehicle or gene editing system comprising said nucleic acid molecule of (a); (c) at least one cell expressing said CAR or a system comprising said CAR, or a population of said cells; and (d) a composition comprising at least one of (a), (b) and (c), for use in a method for treating, preventing, ameliorating, inhibiting or delaying the onset of a pathologic disorder in a mammalian subject. In some embodiments, the CAR molecule comprises the following components: (i) at least one target-binding domain; (ii) at least one transmembrane domain; and (iii) at least one signal transduction domain. It should be understood that this signal transduction domain is capable of relaying, and transducing signals for T cell independent activation.

In some embodiments of the effective amount for use according to the present disclosure, the CAR molecule is as defined by the present disclosure, the system is as defined by the present disclosure, the nucleic acid molecule is as defined by the present disclosure, and the gene editing system is as defined by the present disclosure, the cell is as defined by the present disclosure, and the composition is as defined by the present disclosure.

As indicated herein, the present disclosure provides a CAR-B molecule that allows a cell of the B cell lineage to undergo antigen-induced activation independent of a cell of the T cell lineage. Antigen induced B cell activation begins by the recognition and binding of an antigen by the B cell receptor (BCR), leading to cell proliferation and immunoglobulin class switching. Naturally, this can either take place in a T cell dependent or T cell independent manner.

T cell-dependent B cell activation, as used herein, relates to a process where B cells can obtain help from T cells in the antibody response by acting as antigen-specific antigen presenting cells (APCs). Typical antigens that elucidate a T cell-dependent response (TD antigen) would be foreign soluble protein or hapten conjugated to such proteins, bounded by the surface B cell receptor (BCR). A B cell response to such TD antigen takes multiple days and antibodies generated have a higher affinity and are more functionally versatile than those generated from T cell-independent activation.

The response includes endocytosis of the BCR-antigen complex and subsequent presentation of the degraded antigen on B cell surface as MHC-II-peptide complex which is recognized by T helper (Th) cell's surface TCR. Following TCR-MHC-II-peptide complex formation, T cells express the surface protein CD40L as well as other secreted cytokines, such as IL-4 and IL-21 that serve as necessary co-stimulatory factors for B cell activation by binding corresponding co-stimulatory receptors on B cell surface. T cell-dependent B cell activation promotes B cell proliferation, immunoglobulin class switching, and somatic hypermutation as well as sustains T cell growth and differentiation. These processes are facilitated by T cells within the Germinal Centers (GC) and generate both high-affinity memory B cells and long-lived plasma cells.

Contrary to T-cell dependent B cell activation, in T cell-independent B cell activation, typical antigens that elucidate T cell-independent response (TI antigen) are more diverse and usually exhibit highly repetitive epitopes or contain TLR ligands, such as LPS. TI antigens can induce a humoral response in organisms that lack T cells. Indeed, components of many natural pathogens, such as polysaccharides from bacterial cell walls, viral capsids and unmethylated CpG DNA, can initially elicit TI responses, making TI antigens important mediators of immune defense mechanism against different infections. However, such immune response bypasses the necessity of T cell help that contribute to B cell proliferation, antibody affinity maturation and long-term immune memory, thereby resulting in a more rapid and limited immune response, with antibodies generated that tend to have lower affinity and are less functionally versatile.

Nevertheless, a common, physiologically relevant, naturally occurring pathogen-associated antigen can potentially induce an initial TI response followed by a full and robust TD antibody responses.

T cell independent B cell activation, or antigen induced T cell-like B cell activation, as used herein, in the context of the present invention, results from antigen specific binding to the chimeric antigen receptor (CAR), expressed on surface of the engineered B cell, and induction of B cell activation functions, wherein the CAR molecule comprises an intracellular co-stimulatory signaling domain of at least one receptor naturally capable of relaying T cell-dependent co-stimulatory signals for B cell activation. Such a CAR B engineered cell can mimic intracellular signal transduction for activation by T cells, thereby executing T-cell dependent B cell response without actually being dependent on activation signals coming from T cells themselves and without interacting with any T cell. Thus, in accordance with the present disclosure the CAR molecules disclosed herein specifically allow T cell independent antigen specific and/or antigen induced B cell activation. Still further, it should be understood that in some particular embodiments, B cells may undergo T cell independent activation by some pathogen associated motifs (e.g., LPS). However, this natural response is polyclonal and cannot be considered as an activation of a specific B cell clone. Therefore, in some embodiments, the CARs of the present disclosure allow T cell independent B cell activation of the specific clone of the engineered cells. The CAR is therefore needed for causing the specific activation of desired B cells (the engineered B cells) upon engaging the cognate antigen. Thus, in some embodiments, the T cell independent B cell activation as used herein and mediated by the disclosed CAR molecules, relates to the activation of a specific B cell clone in response to the specific cognate antigen of the CAR. Still further, in some embodiments the T cell independent B cell activation is an activation of a specific B cell clone. In yet some further embodiments the T cell independent B cell activation is any activation caused by the CAR molecules of the invention, with the proviso that the activation is not a polyclonal activation.

Still further, the third component of the CAR molecule of the present disclosure is at least one signal transduction domain. As used herein, the term signal transduction domain, refers in some embodiments to the functional, intracellular portion of a receptor protein that acts to transmit the detected stimulatory information within the cell, thereby regulating the cellular activity through specific signaling pathways. According to some embodiments, this domain is an intracellular domain connected to the transmembrane domain. It some embodiments, the signal transduction domain of the CAR of the present disclosure is at least one intracellular co-stimulatory domain that induces T cell independent B cell activation. In yet some further embodiments, this domain is not derived from a B cell receptor.

Co-stimulatory ligand/receptor: T cell-dependent B cell activation requires reciprocal interactions between T and B cells. This complex interaction between cells presenting (APCs) and recognizing antigens underlays initiation and regulation of adaptive immune responses. It involves the engagement of appropriate antigen cell receptor complexes, co-stimulatory molecules and innate signals as described hereinabove for T cell-dependent B cell activation. Co-stimulatory receptor proteins and corresponding ligands appear to be required for full B and T cell activation/response. Lymphocytes stimulated through the antigen receptor alone fail to produce cytokines, are unable to sustain proliferation, and often undergo apoptosis or become nonresponsive to subsequent stimulation (Frauwirth, K. A., & Thompson, C. B. (2002) The Journal of clinical investigation). Receptors and ligands for co-stimulatory molecules are expressed on B and T lymphocytes and rapidly up-regulated by inflammatory and antigen-specific signals that initiate cell activation. Bidirectional communication between the cells, such that a T cell-B cell interaction can involve mutual co-stimulation and several levels of crosstalk, allowing very specific regulation of lymphocyte activation. The co-stimulatory signals are essential additions to those sent by the antigen receptor and are required for full activation of a lymphocyte. Examples for such co-stimulatory molecule are: (i) MHCII, (ii) CD40, (iii) ICOSL, (iv) CD80/CD86, (v) IL2R, (vi) IL4R, (vii) IL5R, (viii) IL6R, (ix) IL10R, (x) IL12R, (xi) IL13R, (xii) IL21R, (xiii) TACI, (xiv) BCMA, (xv) BAFF-R. Thus, in some embodiments, the intracellular co-stimulatory signal transduction domain comprises at least one of: (i) Major histocompatibility complex class II (MHCII), (ii) Cluster of differentiation 40 (CD40), (iii) Inducible co-stimulatory ligand (ICOSL), (iv) Cluster of differentiation 80/86 (CD80/CD86), (v) interleukin-2 receptor (IL2R), (vi) interleukin-4 receptor (IL4R), (vii) interleukin-5 receptor (IL5R), (viii) interleukin-6 receptor (IL6R), (ix) interleukin-10 receptor (IL10R), (x) interleukin-12 receptor (IL12R), (xi) interleukin-13 receptor (IL13R), (xii) interleukin-21 receptor (IL21R), (xiii) Transmembrane activator and CAML interactor (TACI), (xiv) B-cell maturation antigen (BCMA), (xv) B-cell activating factor receptor (BAFF-R), and any combinations thereof.

More specifically, in some embodiments, the intracellular co-stimulatory signal transduction domain comprises MHCII. As used herein, MHCII, major histocompatibility complex (In humans, encoded by the human leukocyte antigen gene complex (HLA)) is expressed on specialized immune antigen presenting cells (APCs) such as dendritic cells, mononuclear phagocytes and B cells. The complex presents extracellular peptides, mainly those derived from pathogens. Class II MHC molecules interact mainly with immune cells, like the T helper cell (CD4+) expressing T Cell Receptor (TCR). Such recognition with TCR may lead to a full antibody immune response due to activation of B cells.

Still further, in some embodiments, the intracellular co-stimulatory signal transduction domain comprises CD40. As used herein, CD40 is a costimulatory protein found on APCs and B cells and is required for their activation. CD40 activation improves the antigen presentation capacity of B cells. The binding of CD154 (CD40L) on TH cells to CD40 activates antigen presenting cells and induces a variety of downstream effects. B cells stimulated with interleukin-4 and CD40 ligand (CD40L) enhances their proliferation capability.

In some embodiments, the intracellular co-stimulatory signal transduction domain comprises ICOSL. More specifically, ICOSL, the ligand of the T cell co-stimulatory protein ICOS, which is expressed by TH cells and has been implicated in directing T cell differentiation. ICOS has also been consistently linked with the induction of thymus-dependent antibody responses and the germinal center (GC) reaction. ICOS co-stimulation, therefore, involved in dictating adaptive immunity responses and it possess a crosstalk activity related to co-stimulatory molecule CD28 (the receptor for CD80/CD86) (Wikenheiser Daniel J., and Stumhofer Jason S. (2016) Frontiers in Immunology 10; 7:304).

In some further embodiments, the intracellular co-stimulatory signal transduction domain comprises CD80/CD86. More specifically, CD80/CD86, CD80/CD86 (B7-1, B7-2) are type I membrane protein that is a member of the immunoglobulin superfamily. They are expressed on the surface of APCs immune cells and specifically on B cells. They are ligands of the co-stimulatory receptor CD28 that is expressed on T cells. This interaction results in a costimulatory signal in the immunological synapse between the APCs, B-cells and T-cells that result in T and B-cell activation, proliferation and differentiation.

In some embodiments, the intracellular co-stimulatory signal transduction domain comprises IL2R. As used herein, IL2R, is the receptor for Interleukin 2 (IL2) is expressed on T and B lymphocytes. IL2 is secreted mainly by activated T cells and exerts signaling through B cell's IL2R that promotes proliferation of the activated B cell. IL2 signaling crosstalk with other cytokines/stimulatory factors to enhance differentiation of activated B cells. (Tangye Stuart (2014) Frontiers in Immunology, Volume 5, Article 65).

In some embodiments, the intracellular co-stimulatory signal transduction domain comprises IL4R. More specifically, IL4R, the receptor for Interleukin 4. IL4/IL4R co-stimulatory interaction regulates humoral and adaptive immunity responses. Stimulation of the receptor of activated B-cell and T-cell induces several effects of B cell, including stimulation of proliferation, and the differentiation into plasma cell. IL-4 induces B-cell class switching to IgE, and up-regulates MHC class II production (Tangye Stuart (2014) Frontiers in Immunology).

In some embodiments, the intracellular co-stimulatory signal transduction domain comprises IL5R. Specifically, IL5R is the receptor for Interleukin 5. IL5/IL5R co-stimulatory interaction stimulates B cell growth and increases immunoglobulin secretion. It is also a key mediator in eosinophil activation (Tangye Stuart (2014) Frontiers in Immunology).

In some embodiments, the intracellular co-stimulatory signal transduction domain comprises IL6R. IL6R, is the receptor for Interleukin 6. IL6 is a T-cell-derived soluble factor that induce the maturation of B cells into plasma cells through the IL6/IL6R co-stimulatory interaction. IL-6 has important roles in both T- and B-cell activation and survival (Tangye Stuart (2014) Frontiers in Immunology).

In some embodiments, the intracellular co-stimulatory signal transduction domain comprises IL10R. More specifically, IL10R, the receptor for Interleukin 10. IL10 is considered mainly as anti-inflammatory cytokine. Nevertheless, IL10/IL10R co-stimulatory interaction induces several effects on B cell, including stimulation of B cell proliferation and differentiation into plasma cells, class switch recombination and antibody production through the (Tangye Stuart (2014) Frontiers in Immunology).

In some embodiments, the intracellular co-stimulatory signal transduction domain comprises IL12R. More specifically, IL12R, the receptor for Interleukin 12. IL-12 activates human B-cells through the IL-12 receptor (IL-12R) complex (Airoldi I. et. al., (2002) Haematologica) and induces its differentiation into antibody secreting cell. IL12 signaling crosstalk with IL6 signaling (Tangye Stuart (2014) Frontiers in Immunology).

In some embodiments, the intracellular co-stimulatory signal transduction domain comprises IL13R. Specifically, IL13R, the receptor for Interleukin 13. IL13/IL13R co-stimulatory interaction induces several effects on B cell, including stimulation of B cell proliferation, differentiation into plasma cells and class switch recombination (CSR). (Tangye Stuart (2014) Frontiers in Immunology).

In some embodiments, the intracellular co-stimulatory signal transduction domain comprises IL21R. As used herein, IL21R, the receptor for Interleukin 21. IL21/IL21R co-stimulatory interaction induces several effects on B cell, including stimulation of B cell differentiation into plasma cells and class switch recombination (CSR). It is considered as the most potent cytokine regulating B cell functions. IL21 signaling crosstalk with IL4 signaling. (Tangye Stuart (2014) Frontiers in Immunology).

In some embodiments, the intracellular co-stimulatory signal transduction domain comprises BAFF-R. More specifically, BAFF-R, the receptor of BAFF ligand (B cell activating factor) that is expressed by T cells, dendritic cells, monocytes, and macrophages, but not by B cells. BAFF promotes the survival, differentiation and proliferation of B cells and can induce class switch recombination (CSR) and antibody secretion when co-stimulated with BCR and with IL-4, IL-10 and IL-15 (Tangye Stuart (2014) Frontiers in Immunology).

In some embodiments, the intracellular co-stimulatory signal transduction domain comprises TAC. As used herein, TACI, is a surface receptor that can bind the BAFF ligand and APRIL ligand thereby, promoting various aspects of B-cell function (Tangye Stuart (2014) Frontiers in Immunology).

In some embodiments, the intracellular co-stimulatory signal transduction domain comprises BCMA. More specifically, BCMA, is a surface receptor that can bind the BAFF ligand and APRIL ligand thereby, promoting various aspects of B-cell function (Tangye Stuart (2014) Frontiers in Immunology).

The present disclosure provides a CAR molecule. Chimeric Antigen Receptor (CAR), —as used herein, refers to a recombinant polypeptide comprising at least an extracellular antigen binding domain, a transmembrane domain and an intracellular cytoplasmic signaling domain comprising a functional stimulatory domain. The receptors are chimeric because they couple between extracellular antigen-binding capabilities and intracellular T- or B-cell activating functions, in a single receptor molecule. CARs have been engineered to give the B or T cells they are expressed in the new ability to recognize a specific antigen of interest, thereby facilitating an immune reaction against it. For example, the technology is used in immunotherapy for specifically recognizing specific cancer cells' antigens of interest in order to more effectively direct the immune cells towards those target cells and destroy them.

The invention provides chimeric antigen receptors (CARs) that allow a cell of the B cell lineage to undergo antigen-induced activation independent of a cell of the T cell lineage. The invention therefore further provides methods for activating engineered B cells for antigen-induced secretion of antibodies of interest, in a T cell independent manner. The CAR molecule provided herein, comprises at least one target-binding domain, that may be in some embodiments, any target-recognition element, for example, at least one antibody or any antigen-binding fragments or domains thereof, as discussed herein above. In yet some further embodiments, the target-recognition element of the CAR molecule of the present disclosure comprises at least one antibody or any antigen-binding fragment/s, portion/s or chimera/s thereof.

Exemplary categories of antigen-binding domains that can be used in the context of the present invention include antibodies, antigen-binding portions of antibodies (e.g., single chain variable fragments (scFv)), peptides that specifically interact with a particular antigen (e.g., peptibodies), receptor molecules that specifically interact with a particular antigen, proteins comprising a ligand-binding portion of a receptor that specifically binds a particular antigen or antigen-binding scaffolds. The antigen binding domains in accordance with the invention may recognize and bind a specific antigen or epitope. It should be therefore noted that the term “binding specificity”, “specifically binds to an antigen”, “specifically immuno-reactive with”, “specifically directed against” or “specifically recognizes”, when referring to an antigen or particular epitope, refers to a binding reaction which is determinative of the presence of the epitope in a heterogeneous population of proteins and other biologics. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three-dimensional structural characteristics as well as specific charge characteristics. Still further, as indicated above, an “antigen-binding domain” can comprise or consist of an antibody or antigen-binding fragment of an antibody such as single chain variable fragments (scFv). The term “antibody” as used herein, means any antigen-binding molecule or molecular complex comprising at least one complementarity determining region (CDR) that specifically binds to or interacts with a particular antigen or any epitope thereof. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

A typical antibody is composed of two immunoglobulin (Ig) heavy chains and two Ig light chains. In humans, antibodies are encoded by three independent gene loci, namely the immunoglobulin heavy locus (IgH) on chromosome 14, containing the gene segments for the immunoglobulin heavy chain, the immunoglobulin kappa (κ) locus (IgK) on chromosome 2, containing the gene segments for part of the immunoglobulin light chain and the immunoglobulin lambda (λ) locus (IgL) on chromosome 22, containing the gene segments for the immunoglobulin light chain.

The antibody and BCR heavy chains comprise 51 Variable (V) gene segments, 27 Diversity (D) gene segments, 6 Joining (J) gene segments. The antibody and BCR light chains comprise 40 Vκ, 31 Vλ, 5 Jκ, 4 Jλ gene segments.

Still further, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

Single domain antibodies also known as nanobodies have previously obtained by immunizing dromedaries, camels, llamas, alpacas, sharks, murine, rabbits and humans)

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

Single chain variable fragments (scFv) comprise the variable regions of the heavy (VH) and light chains (VL) of immunoglobulins, connected with a short linker peptide. Single-chain variable fragments lack the constant Fc region found in complete antibody molecules. Nevertheless, scFv retains the specificity of the original immunoglobulin, despite removal of the constant regions and the introduction of the linker.

The antibody suitable for the invention may also be a bi-specific antibody (such as Bi-specific T-cell engagers-BiTEs) or a tri-specific antibody.

The antibody suitable for the invention may also be a variable new antigen receptor antibody (V-NAR). VNARs are a class of small, immunoglobulin-like molecules from the shark immune system. Humanized versions of VNARs could be used to bind protein epitopes that are difficult to access using traditional antibodies.

It should be understood that an antibody of interest or any antigen-binding fragments thereof, forming the, or comprised within, the target-binding domain of the CAR-B provided in accordance with the invention may be directed to any antigen of interest, specifically any antigen specific for a pathologic disorder. In more specific embodiments, the antibody of interest may be directed against antigens specific for proliferative disorders, specifically, tumor associated antigens (TAAs), or antigens specific for any pathogen, specifically, viral, bacterial, fungal or parasitic pathogen. Specific pathogens applicable in the present invention are described in more detail herein after.

In some specific embodiments, the CAR of the present disclosure may comprise a target recognition element that may be at least one antibody directed against at least one of a viral antigen and a tumor associated antigen (TAA).

Tumor or cancer associated antigen (TAA), as used herein may be an antigen that is specifically expressed, over expressed or differentially expressed in tumor cells. In yet some further embodiments, TAA can stimulate tumor-specific T-cell immune responses. Exemplary tumor antigens that may be applicable in the present invention, include, but are not limited to, RAGE-1, tyrosinase, any MAGE, specifically, MAGE-1, MAGE-2, NY-ESO-1, Melan-A/MART-1, glycoprotein (gp) 75, gp100, gp130, MUC1, beta-catenin, PRAME, MUM-1, WT-1, CEA, PR-1 CD45, glypican-3, IGF2B3, Kallikrein4, KIF20A, Lengsin, Meloe, MUC5AC, survivin, CLPP, Cyclin-A1, SSX2, XAGE1b/GAGED2a, MAGE-A3, MAGE-A6, LAGE-1, CAMEL, hTRT and Eph. and TRP-1. Still further, TAA may be recognized by CD8+ T cells as well as CD4+ T cells. Non limiting examples of TAA recognized by CD8+ T cells may be CSNK1A1, GAS7, HAUS3, PLEKHM2, PPP1R3B, MATN2, CDK2, SRPX (P55L), WDR46 (T227I), AHNAK (S4460F), COL18A1 (S126F), ERBB2 (H197Y), TEAD1 (L209F), NSDHL (A290V), GANAB (S184F), TRIP12 (F1544S), TKT (R438W), CDKN2A (E153K), TMEM48 (F169L), AKAP13 (Q285K), SEC24A (P469L), OR8B3 (T190I), EXOC8 (Q656P), MRPS5 (P59L), PABPC1 (R520Q), MLL2, ASTN1, CDK4, GNL3L, SMARCD3, MAGE-A6, MED13, PAS5A WDR46, HELZ2, AFMID, CENPL, PRDX3, FLNA, KIF16B, SON, MTFR2 (D626Y), CHTF18 (L769V), MYADM (R30W), NUP98 (A359D), KRAS (G12D), CASP8 (F67V), TUBGCP2 (P293L), RNF213 (N1702S), SKIV2L (R653H), H3F3B (A48T), AP15 (R243Q), RNF10 (E572K), PHLPP1 (G566E) and ZFYVE27 (R6H). Non limiting examples of TAA recognized by CD4+ T cells may be ERBB2IP (E805G), CIRH1A (P333L), GART (V551A), ASAP1 (P941L), RND3 (P49S), LEMD2 (P495L), TNIK (S502F), RPS12 (V104I), ZC3H18 (G269R), GPD2 (E426K), PLEC (E1179K), XPO7 (P274S), AKAP2 (Q418K) and ITGB4 (S10021). Non-limiting examples of MHC class II-restricted antigens may be Tyrosinase, gp100, MART-1, MAGE-A1, MAGE-A2, MAGE-A3, MAGE-A6, LAGE-1, CAMEL, NY-ESO-1, hTRT and Eph.

Cancer antigen and tumor antigen are used interchangeably herein. The antigens may be related to cancers that include, but are not limited to, Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma and any associated TAAs, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer).

Still further, in some embodiments, a few examples of antibodies used in the treatment of cancer that may be applicable in the present invention include, but are not limited to monoclonal antibodies such as Bevacizumab (UNII: 2S9ZZM9Q9V), Cetuximab (UNII: PQX0D8J21J), Panitumumab (UNII: 6A901E312A), Rituximab (UNII: 4F4X42SYQ6), Alemtuzumab (UNII: 3A189DH42V), Ipilimumab (UNII: 6T8C155666, Yervoy), that is a check point inhibitor, specifically, a monoclonal antibody that works to activate the immune system by targeting CTLA-4, Tremelimumab (UNII: P188ANX8CK, formerly ticilimumab, CP-675,206) is a fully human monoclonal antibody against CTLA-4, ibritumomab tiuxetan (UNII: 4Q52C550XK), lambrolizumab (formerly MK-3475, Pembrolizumab, Keytruda® UNII: DPT003T46P), that is a check point inhibitor, specifically, a humanized antibody that targets programmed cell death (PD-1), Nivolumab (Opdivo® UNII: 31YO63LBSN) is an Fab fragment of an antibody that binds the extracellular domain of PD-1, Atezolizumab (trade name Tecentriq) is a fully humanized, engineered monoclonal antibody of IgG1 isotype against the protein programmed cell death-ligand 1 (PD-L1), Avelumab (trade name Bavencio) is a fully human monoclonal antibody that targets PD-L1, Durvalumab is a human immunoglobulin G1 kappa (IgG1κ) monoclonal antibody that blocks the interaction of PD-L1 with the PD-1 and CD80 (B7.1) molecules and Tremelimumab discussed above (formerly ticilimumab; UNII: QEN1X95CIX) that is a check point inhibitor and ado-trastuzumab emtansine (UNII: SE2KH7T06F). In some specific embodiments, the antibody of interest may be an antibody or BCR directed against a viral antigen. It should be appreciated that any of the viral pathogens discussed herein after, is applicable in this aspect, as well as in all aspects of the invention.

In some particular embodiments, CARs, the co-expressed BCRs, and/or antibodies (used herein as the target-binding domain of the CAR) applicable in the methods, nucleic acid molecules, systems, cells and compositions of the invention may be directed against any antigen derived from a pathogen, specifically, viral, bacterial, fungal, parasitic pathogen and the like. In some specific embodiments, the viral pathogen may be of any of the following orders, specifically, Herpesvirales (large eukaryotic dsDNA viruses), Ligamenvirales (linear, dsDNA (group I) archaean viruses), Mononegavirales (include nonsegmented (−) strand ssRNA (Group V) plant and animal viruses), Nidovirales (composed of (+) strand ssRNA (Group IV) viruses), Ortervirales (single-stranded RNA and DNA viruses that replicate through a DNA intermediate (Groups VI and VII)), Picornavirales (small (+) strand ssRNA viruses that infect a variety of plant, insect and animal hosts), Tymovirales (monopartite (+) ssRNA viruses), Bunyavirales contain tripartite (−) ssRNA viruses (Group V) and Caudovirales (tailed dsDNA (group I) bacteriophages).

In yet some further specific embodiments, the antibodies acting as a target recognition element in the CAR s of the present disclosure may be specifically directed against DNA viruses, specifically, any virus of the following families: the Adenoviridae family, the Papovaviridae family, the Parvoviridae family, the Herpesviridae family, the Poxviridae family, the Hepadnaviridae family and the Anelloviridae family.

In yet some further specific embodiments, the antibodies used as a target recognition element in the CAR s of the present disclosure may be specifically directed against RNA viruses, specifically, any virus of the following families: the Reoviridae family, Picornaviridae family, Caliciviridae family, Togaviridae family, Arenaviridae family, Flaviviridae family, Orthomyxoviridae family, Paramyxoviridae family, Bunyaviridae family, Rhabdoviridae family, Filoviridae family, Coronaviridae family, Astroviridae family, Bornaviridae family, Arteriviridae family, Hepeviridae family and the Retroviridae family.

In more specific embodiments, the antibody of interest may be directed against any antigen derived from a viral pathogen of the order Mononegavirales. In yet some further embodiments, the antibody of interest may be directed against an antigen derived from a virus of the family Pneumoviridae. In more specific embodiments antibody or BCR of interest may be directed against any antigen derived from a viral pathogen of the genus Orthopneumovirus. In some specific embodiments, such viral antigen may be an antigen specific for respiratory syncytial virus (RSV), for example, any one of the Human respiratory syncytial virus (HRSV), A2 and B1, the bovine respiratory syncytial virus (BRSV) and the murine pneumonia virus (MPV). In more specific embodiments, the antibody of interest may be directed against the human RSV. In yet some further specific embodiments, the anti-RSV antibody may be the anti-RSV palivizumab antibody. More specifically, Palivizumab (brand name Synagis, manufactured by MedImmune) is a humanized monoclonal antibody (IgG) directed against an epitope in the A antigenic site of the F protein of RSV.

In yet some further specific embodiments, the antibody of interest may be directed against any antigen derived from a viral pathogen of the family Retroviridae. In yet some further embodiments, the antibody of interest may be directed against an antigen derived from a virus of the subfamily Orthoretrovirinae. In more specific embodiments antibody of interest may be directed against any antigen derived from a viral pathogen of the genus Lentivirus, specifically, of the species human immunodeficiency virus (HIV). In yet some further embodiments, the antibody of interest in accordance with some embodiments of the invention may be an anti-HIV-1 antibody. In yet some further specific embodiments the antibody of interest may be the anti-HIV 3BNC117 antibody. More specifically, the 3BNC117, as used herein, is a neutralizing antibody (bNAb) directed against the CD4 binding site of HIV-1 Env, specifically, an epitope on the pg120 protein.

Specific embodiments that relate to particular viruses associated with specific disorders are specified herein below. It should be understood that any of the viral pathogens and any of the bacterial, fungal and parasite pathogen described herein after, are also applicable in connection with the antigens derived therefrom that are recognized by the target-recognition element of the CAR-B of the invention, for example, antibodies or any antigen-binding fragments thereof.

A further aspect of the present disclosure relates to a genetically engineered cell of the B cell lineage expressing at least one CAR molecule, or any population of cells comprising at least one of the genetically modified cell/s of the present disclosure. The engineered cell of the present disclosure is capable of undergoing antigen-induced activation independent of a cell of the T cell lineage. In yet some further embodiments, the CAR molecule of the engineered cell/s of the present disclosure comprises: (i) at least one target-binding domain; (ii) at least one transmembrane domain; and (iii) at least one signal transduction domain. It should be noted that the signal transduction domain of the CAR of the engineered cell/s of the present disclosure is capable of relaying signals for T cell independent activation. In some embodiments, the engineered cell/s of the present disclosure further express at least one effector protein. More specifically, such effector protein is at least one of: (i) at least one membranal and/or a secreted antibody or antibody derivative; (ii) at least one Bi-specific T cell engager; (iii) at least one cytokine; (iv) at least membranal receptor; and (v) at least one secreted toxin. In some embodiments, the engineered cell of the present disclosure may comprise at least one engineered BCR.

In some embodiments of the present invention, at least one effector protein is co-expressed with the CAR molecule in the same cell of the B lineage, specifically, B cell. An effector protein in the context of the present invention refers to any protein engineered to be co-expressed with the CAR molecule in the same B cell, exerting a pre-defined desired effect from said B cell. The effector protein is at least one protein defined from the following unlimiting list of effector proteins: (i) membranal and/or a secreted antibody or antibody derivative (ii) Bi-specific T cell engager, (iii) a cytokine, (iv) a membranal receptor and (v) a secreted toxin.

In some embodiments the effector protein is a—(iii) a cytokine or (v) a secreted toxin—that can exert its effect in an autonomous manner, independent of the effect exerted by the antigen induced activation of the engineered CAR molecule on the functions of the activated cell of the B lineage.

In some embodiments the effector protein is a—(i) membranal and/or a secreted antibody or antibody derivative or (ii) Bi-specific T cell engager—that exert an effect that is dependent/related/affected by the activation of the cell by the CAR.

In some embodiments the effector protein is—(iv) a membranal receptor.

More specifically, in some specific embodiments, the cell of the invention is a B cell. More specifically, the mammalian cells of the B cell lineage provided by the invention may be any lymphocytes of the B lineage. The engineered B cells disclosed herein are also provided by the invention. B cells are derived from the bone marrow, originating from hematopoietic stem cells, which differentiate into multipotent progenitor cells then into common lymphoid progenitor cells. The subsequent developmental process of B cells is complex with many different stages, which is dependent upon the stimuli received and through which the B cell gains its antigen specificity. At these stages of development different surface antigens are expressed enabling detection of specific B cells during their maturation process. For example, CD19, CD24 and CD72 are pan B cell markers that are expressed on all B-lineage cells throughout their different stages of differentiation. “Lymphocytes” are mononuclear nonphagocytic leukocytes found in the blood, lymph, and lymphoid tissues. They are divided on the basis of ontogeny and function into two classes, B and T lymphocytes, responsible for humoral and cellular immunity, respectively. Most are small lymphocytes 7-10 μm in diameter with a round or slightly indented heterochromatic nucleus that almost fills the entire cell and a thin rim of basophilic cytoplasm that contains few granules. When “activated” by contact with antigen, small lymphocytes begin macromolecular synthesis, the cytoplasm enlarges until the cells are 10-30 μm in diameter, and the nucleus becomes less completely heterochromatic; they are then referred to as large lymphocytes or lymphoblasts. These cells then proliferate and differentiate into B and T memory cells and into the various effector cell types: B cells into plasma cells and T cells into helper, cytotoxic, and suppressor cells.

Still further, in some embodiments, the cells provided by the invention are applicable in the methods and compositions of the invention may be a B cell progenitor. B cells develop from hematopoietic stem cells (HSCs) that originate from bone marrow. Their development into B cells occurs in several stages, each marked by various gene expression patterns and immunoglobulin H chain and L chain gene loci arrangements, the latter due to B cells undergoing V(D)J recombination as they develop.

To ensure proper development, B cells undergo two types of selection while developing in the bone marrow. Positive selection occurs through antigen-independent signaling involving both the pre-BCR and the BCR. If these receptors do not bind to their ligand, B cells do not receive the proper signals and cease to develop. Negative selection occurs through the binding of self-antigen with the BCR; if the BCR can bind strongly to self-antigen, then the B cell undergoes one of four fates: clonal deletion, receptor editing, anergy, or ignorance (B cell ignores signal and continues development). This negative selection process leads to a state of central tolerance, in which the mature B cells does not bind with self-antigens present in the bone marrow.

The development process in the bone marrow occurs in germinal Centers. B cell lymphopoiesis in the bone marrow is as follows: Pro-B cells, Pre-B-I cells, Pre-B-II large cells, Pre-B-II small cells and Immature B cells. To complete development, Immature B cells migrate from the bone marrow to the spleen as well as pass through two transitional stages: T1 and T2. Throughout their migration to the spleen and after spleen entry, they are considered T1 B cells. Within the spleen, T1 B cells transition to T2 B cells. T2 B cells differentiate into either follicular (FO) B cells or marginal zone (MZ) B cells depending on signals received through the BCR and other receptors. Once differentiated, they are now considered mature B cells, or naive B cells. While immature and during the T1 phase, B cells express BCR of class IgH, but BCR expression changes to the classes IgM and IgD after transition into the T2 phase and while mature up to activation. In yet some further embodiments, the cells provide by the invention, expressing the CAR-B discussed herein, administered to the subject by the method of the invention may be splenocytes of any subsect, or any lymphocytes obtained or present in lymph nodes and bone marrow.

In some embodiments, the engineered B cells of the invention may be primary B cells.

Primary B cells are cells that were obtained for culture directly from a subject. In contrast, secondary cell cultures are obtained from an already established primary culture. Still further, in some embodiments, the engineered B cells of the invention may be B cells of a B cell line, specifically immortalized B cells. Immortalized B cells are a population of cells from a multicellular organism which would normally not proliferate indefinitely but, due to mutation, have evaded normal cellular senescence and instead can keep undergoing division. These B cells can therefore be grown for prolonged periods in vitro. An example of immortalized B cells are Epstein-Barr virus (EBV)-immortalized B cells.

It should be understood that the B cells as defined herein are applicable for any of the methods, compositions, systems, or any aspect of the invention. It should be appreciated that the engineered B cell provided by the invention enables the genetically engineered BCR to be subjected to somatic hypermutation (SHM) and affinity maturation, as well as class switch recombination (CSR also called isotype switch) and memory retention. Moreover, the engineered B cells of the invention retain the ability of homing to germinal centers in a mammalian subject.

More specifically, Homing is the phenomenon whereby cells migrate to the organ of their origin. By homing, transplanted hematopoietic cells are able to travel to and engraft or establish residence in the bone marrow. Various chemokines and receptors are involved in the homing of hematopoietic stem cells. Lymphocyte homing refers to adhesion of the circulating lymphocytes in blood to specialized endothelial cells within lymphoid organs. These diverse tissue-specific adhesion molecules on lymphocytes (homing receptors) and on endothelial cells (vascular addressins) contribute to the development of specialized immune responses. Naive lymphocytes are able to circulate into secondary lymphoid tissues, Peyer's patches, lymph nodes, and the spleen. Because they have not yet been exposed to antigen, these lymphocytes are undifferentiated and express few homing receptors. High endothelial venules (HEVs) are cells found in secondary lymphoid organs that express large quantities of cell adhesion molecules, enabling undifferentiated lymphocytes to bind. After entering lymph nodes and Peyer's patches via HEVs, naive T and B cells are exposed to antigen circulating in lymph and differentiate to contribute to the adaptive immune response. HEVs develop from cytokine production after exposure to antigen and express adhesion molecules from the selectin family, mucin-like family, and the Ig superfamily. Mature lymphocytes are constantly recirculating in the blood and can traffic to secondary lymphoid tissue as well as target tissue including mucosal tissues of the lamina propria, inflammation, and other extralymphoid immune effector sites. Lymphocyte homing receptor expression is altered by antigen exposure. This function enables the adaptive immune system to specialize an immune response in different parts of the body. Upon exposure to antigens, lymphocytes lack homing ability during a period of sessile differentiation and cell division, and antigen specific lymphocytes are stored in the spleen for 1-3 days. Subsequently, antigen-stimulated B and T cells express homing receptors particularly for the HEV in initial site of immunization tissue. Furthermore, lymphocytes can alter cell adhesion molecule “activatability” to increase binding ability. Organ-specific lymphocyte homing is important for antigen-specificity and in avoiding autoimmune cross-reactions.

The germinal center (GC) is a specialized microenvironment formed within the B cell follicles of secondary lymphoid tissues upon infection or immunization. The GC is divided into two distinct compartments. The dark zone (DZ) that contains a network of CXCL12-producing reticular cells (CRCs) and is the site of GC B cell proliferation and somatic hypermutation (SHM). Centroblasts then follow a CXCL13 gradient to enter the light zone (LZ) as centrocytes through their expression of CXCR5. In the LZ, centrocytes capture antigen presented on follicular dendritic cells (FDCs) which they internalize, process and subsequently present to T follicular helper (Tfh) cells in order to undergo selection. This process is regulated by T follicular regulatory (Tfr) cells which are also present in the LZ. Upon receiving survival signals from Tfh cells, centrocytes re-enter the DZ for further rounds of proliferation and SHM after which they exit the GC as memory B cells or high-affinity antibody-secreting plasma cells.

Affinity maturation is the process by which Follicular B helper T cells (Tfh) activated B cells produce antibodies with increased affinity for antigen during the course of an immune response. With repeated exposures to the same antigen, a host will produce antibodies of successively greater affinities. A secondary response can elicit antibodies with several fold greater affinity than in a primary response. Affinity maturation primarily occurs on surface immunoglobulin of germinal center B cells and as a direct result of somatic hypermutation (SHM) and selection by Tfh cells.

The process involves two interrelated processes, occurring in the germinal centers of the secondary lymphoid organs:
Somatic hypermutation: Mutations in the variable, antigen-binding coding sequences of the immunoglobulin genes.
Clonal selection: B cells that have undergone SHM must compete for limiting growth resources, including the availability of antigen and paracrine signals from Tfh cells. The follicular dendritic cells (FDCs) of the germinal centers present antigen to the B cells, and the B cell progeny with the highest affinities for antigen, having gained a competitive advantage, are favored for positive selection leading to their survival. Positive selection is based on steady cross-talk between Tfh cells and their cognate antigen presenting GC B cell. Because a limited number of Tfh cells reside the germinal center, only highly competitive B cells stably conjugate with Tfh cells and thus receive T cell-dependent survival signals. B cell progeny that undergone SHM, but bind antigen with lower affinity will be out-competed, and be deleted. Over several rounds of selection, the resultant secreted antibodies produced will have effectively increased affinities for antigen.

Immunological memory is the ability of the immune system to quickly and specifically recognize an antigen that the body has previously encountered and initiate a corresponding immune response. Generally, these are secondary, tertiary and other subsequent immune responses to the same antigen. Immunological memory is responsible for the adaptive component of the immune system i.e., the memory T and B cells. Immunological memory is the basis of vaccination.

Memory B cells are plasma cells that are able to produce antibodies for a long time. Unlike the naive B cells involved in the primary immune response the memory B cell response is slightly different. The memory B cell has already undergone clonal expansion and differentiation and affinity maturation, so it is able to divide multiple times faster and produce antibodies with much higher affinity (especially IgG). Memory B cell activity in secondary lymphatic organs is highest during the first 2 weeks after infection. Subsequently, after 2 to 4 weeks its response declines. After the germinal center reaction the memory plasma cells are located in the bone marrow which is the main site of antibody production within the immunological memory.

As indicated above, the invention provides CAR molecules that allow T antigen-induced activation independent of T cell, genetically engineered B cells that express these CAR molecules and thereby undergo T cell independent activation, as well as genetically engineered B cell receptors that may be co-expressed with the disclosed CAR molecules and methods for preparations thereof. The B-cell receptor or BCR is a transmembrane receptor protein located on the outer surface of B cells. The B-cell receptor is composed of two elements, specifically, (i) a membrane-bound immunoglobulin molecule of one isotype (IgD, IgM, IgA, IgG, or IgE) with the exception of the presence of an integral membrane domain, these are identical to their secreted forms; and (ii) a signal transduction moiety composed of a heterodimer called Ig-α/Ig-β (CD79), bound together by disulfide bridges. Each member of the dimer spans the plasma membrane and has a cytoplasmic tail bearing an immuno-receptor tyrosine-based activation motif (ITAM). In some embodiments, the polypeptide provided by the invention that are co-expressed with the CAR-B provided herein, may be any of the genetically engineered BCRs disclosed herein, or any derivatives, variants or fragments thereof, as well as any antibody derived therefrom. It should be thus understood that the invention therefore encompasses any CAR and/or BCR disclosed by the invention, as well as any antibody derived therefrom. Moreover, the invention encompasses any CAR-B, any co-expressed BCR prepared by any of the methods of the invention as well as any antibody derived from such BCRs, and any CAR, BCR or derived antibody encoded by any of the nucleic acid molecules of the invention as disclosed herein.

The present disclosure provides CAR-B molecules that are composed of amino acid residues and are therefore a polypeptide. The term “polypeptide” as used herein refers to amino acid residues, connected by peptide bonds. A polypeptide sequence is generally reported from the N-terminal end containing free amino group to the C-terminal end containing free carboxyl group and may include any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that contains portions that occur in nature separately from one another (i.e., from two or more different organisms, for example, human and non-human portions). In some embodiments, a polypeptide has an amino acid sequence that is engineered in that it is designed and/or produced through action of the hand of man. More specifically, “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide. Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidation, carboxylation, sulfhydryl bond formation, cleavage and the like.

It should be appreciated that the invention encompasses the use of any variant or derivative of the polypeptides of the invention, specifically any polypeptide comprising at least one of the amino acid sequences as denoted by any one of SEQ ID NOs: 12, 13, 14, 15 and 16, or any derivatives thereof, and any polypeptides that are substantially identical or homologue to the polypeptides encoded by the nucleic acid sequence of the invention, as indicated herein above. The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof. Proteins orthologs or homologues having a sequence homology or identity to the proteins of interest in accordance with the invention, specifically, receptors, chimeras and antibodies described herein, may share at least 50%, at least 60% and specifically 65%, 70%, 75%, 80%, 85%, 90%, 91%, 92%, 93%, 94%, 95%, 96%, 97%, 98%, 99%, or higher, specifically as compared to the entire sequence of the proteins of interest in accordance with the invention, specifically, any one of SEQ ID NOs: 12, 13, 14, 15 and 16, or any derivatives thereof.

In some embodiments, derivatives refer to polypeptides, which differ from the polypeptides specifically defined in the present invention by insertions, deletions or substitutions of amino acid residues. It should be appreciated that by the terms “insertion/s”, “deletion/s” or “substitution/s”, as used herein it is meant any addition, deletion or replacement, respectively, of amino acid residues to the polypeptides disclosed by the invention as indicated above, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N′ or C′ termini thereof. With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention. For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:

1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

Variants of the polypeptides of the invention may have at least 80% sequence similarity or identity, often at least 85% sequence similarity or identity, 90% sequence similarity or identity, or at least 95%, 96%, 97%, 98%, or 99% sequence similarity or identity at the amino acid level, with the protein of interest, such as the various polypeptides of the invention. It should be understood that the percentage of similarity or identity refer to the similarity or identity to the entire sequences as denoted by any one of SEQ ID NO. identifiers.

The invention involves the provision of a nucleic acid molecule encoding the disclosed CAR, that in some embodiments may be, and/or comprised within, a cassette, that is used in the methods, cells, compositions and uses described in all aspects of the invention. The term “nucleic acid cassette” refers to a polynucleotide sequence comprising at least one regulatory sequence operably linked to a sequence encoding a nucleic acid sequence encoding the CAR-Bs disclosed herein. All elements comprised within the cassette of the invention are operably linked together. The term “operably linked”, as used in reference to a regulatory sequence and a structural nucleotide sequence, means that the nucleic acid sequences are linked in a manner that enables regulated expression of the linked structural nucleotide sequence.

By an “2A peptide sequence”, it is meant a nucleotide sequence that allows for the initiation of protein translation in the middle of a messenger RNA (mRNA) sequence. More specifically, a 2A peptide sequence or a CHYSEL site causes a eukaryotic ribosome to release the growing polypeptide chain, but continue translating, thereby giving rise to two separate polypeptides from a single translating ribosome. An expression cassette using a 2A peptide may be therefore used for two or more nucleic acid sequences encoding the CAR-Bs disclosed herein. In some embodiments, this sequence may be used to separate the coding region of two or more polypeptides encoded by two or more nucleic acid sequences encoding the CAR-Bs disclosed herein. As a non-limiting example, the sequence encoding the 2A peptide may be between a first coding region and a second coding region. In other embodiments, the 2A peptide may be used in the polynucleotides of the present invention to produce two, three, four, five, six, seven, eight, nine, ten or more proteins, or any other product of the nucleic acid sequence encoding the CAR-Bs disclosed herein provided by the invention. In certain embodiments, non-limiting example for 2A-peptide that may be used by the invention may be the Picornaviruse 2A peptide (P2A).

As used herein, a “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present invention.

In some embodiments, promoters applicable in the present invention may be either inducible or constitutive. In yet some further embodiments, a functional fragment of a promoter applicable in the methods and nucleic acid molecules encoding the CAR-B, or any cassettes of the invention may be a minimal promoter. The term “minimal promoter” includes partial promoter sequences that define the start site of transcription for the linked sequence to be transcribed which by itself is not capable of initiating transcription. Thus, the activity of such a minimal promoter is dependent upon the binding of a transcriptional activator to an operatively linked regulatory sequence, e.g., enhancer. In certain embodiments a minimal promoter may be included in the nucleic acid molecules or cassettes of the invention. A “constitutive promoter” refers to a promoter that allows for continual transcription of the coding sequence or gene under its control. In yet some further embodiments, a promoter suitable in the nucleic acid molecules, vectors and/or cassette of the invention may be an inducible promoter. An “inducible promoter” refers to a regulatory region that is operably linked to one or more genes, wherein expression of the gene(s) is increased in the presence of an inducer of said regulatory region. An “inducible promoter” refers to a promoter that initiates increased levels of transcription of the coding sequence or gene under its control in response to a stimulus or an exogenous environmental condition. It should be appreciated that the promoters suitable for the present invention may be either endogenous or heterologous. The phrase “endogenous promoter” includes a promoter that is naturally associated, e.g., in a wild-type organism, with an endogenous gene. Thus, in some specific embodiments, the nucleic acid molecules, vectors and/or cassette of the invention may comprise or operably liked to an endogenous promoter, for example, the endogenous promoter of the Ig heavy chain or the Ig light chain. It should be appreciated that such endogenous promoter may be either ectopically added or may be used in its original endogenous location.

In yet some further embodiments, the nucleic acid molecules, vectors and/or cassette/s of the invention may comprise heterologous promoter. The term “heterologous” includes a promoter from a different source or gene. It should be understood that in some embodiments, a promoter comprised within the nucleic acid molecules, vectors and/or nucleic acid cassette of the invention may be located 5′ to the nucleic acid sequence encoding the CAR-Bs disclosed herein. In some embodiments, relevant promoters that may be used by the methods, nucleic acid molecules, vectors and/or cassettes of the invention may include but are not limited to CMV promoter, SFFV promoter, EF1alpha promoter, AAT promoter, BgH promoter and any appropriate promoter.

In yet some further embodiments, the nucleic acid molecules encoding the CAR provided by the invention and by the methods and compositions of the invention may further comprise at least one degron sequence. Degrons are readily understood by the skilled artisan as amino acid sequences that control the stability of the protein of which they are part. In some embodiments, a suitable degron comprised within the nucleic acid molecules encoding the CAR of the invention may be constitutive. In yet some further embodiments, the degron may exerts its influence on protein in an inducible manner. In some embodiments, the degron sequence may be located 5′ to the nucleic acid sequence encoding the CAR-Bs disclosed herein. In yet some further embodiments, the nucleic acid molecules encoding the CAR provided by the invention and by the methods and compositions of the invention may comprise at least one signal peptide leader. “Signal peptide leader”, as used herein, shall mean a peptide chain (of about 3-60 amino acids long) that directs the post-translational transport of a protein to the endoplasmic reticulum and may be cleaved off. In some embodiments, the signal peptide may be located 5′ to the nucleic acid sequence encoding the CAR-Bs disclosed herein. In some further embodiments, the nucleic acid molecules encoding the CAR provided by the invention and by the methods and compositions of the invention may comprise at least one mRNA stabilizing sequence. As used herein, a mRNA stabilizing sequence refers to a nucleic acid sequence that enables to extend the life-time of a mRNA strand. Non limiting examples of mRNA stabilizing elements may include Polyadenylation, 3′ untranslated regions (3′-UT) such as histone mRNA 3′-terminal stem-loop, AU-rich elements (AUREs), Iron-responsive element and Long-range stem loop of insulin-like growth factor II (IGF II), mRNA cap. In some embodiments, the mRNA stabilizing sequence may be located 3′ to the nucleic acid sequence encoding the CAR-Bs disclosed herein. In yet some further embodiments, the nucleic acid molecules, vectors and/or cassette provided by the invention and by the methods and compositions of the invention may comprise at least one stop codon. A stop codon (or termination codon) is a nucleotide triplet within messenger RNA that signals a termination of translation into proteins. Stop codons signal the termination of this process by binding release factors, which cause the ribosomal subunits to disassociate, releasing the amino acid chain. There are three different stop codons in RNA; UAG (“amber”), UAA (“ochre”), UGA (“opal”), in DNA; TAG (“amber”), TAA (“ochre”), TGA (“opal” or “umber”). It should be noted that in some embodiments, the stop codon may be located 3′ to the nucleic acid sequence encoding the CAR-Bs disclosed herein. In yet some further embodiments, nucleic acid molecules, vectors and/or the cassette provided by the invention and by the methods and compositions of the invention may comprise at least one 3-frame stop codon sequence. More specifically, the nucleic acid molecules encoding the CAR disclosed herein may comprise protein translation stop codons in each frame of translation, so that translation from the transcripts of any nucleic acid sequence encoding the CAR-Bs disclosed herein is halted at the point of insertion. Each translation stop sequence (known henceforth as a “3 frame stop codon sequence”) carries stop codons in all 3 frames of translation. In some embodiments, the 3 frame stop codon sequence may be located 5′ to the nucleic acid sequence encoding the CAR-Bs disclosed herein.

Still further, in certain embodiments, the nucleic acid molecules encoding the CAR provided by the invention and by the methods and compositions of the invention may further comprise a nucleic acid sequence encoding at least one protein stabilizing sequence. A protein stabilizing sequence relates to an amino acid sequence useful for stabilization of otherwise unstable proteins, particularly proteolytically sensitive proteins. The stabilization sequence may include a limited number of amino acids ranging from about ten to about 50 residues. The amino acids is such that the secondary and tertiary structure assumes the form of an outwardly directed, properly aligned hydrophobic face and a positively charged polar face. In some embodiments, the protein stabilizing sequence may be located 5′ to the nucleic acid sequence encoding the CAR-Bs disclosed herein. Still further, in some embodiments, the nucleic acid molecules encoding the CAR provided by the invention and by the methods and compositions of the invention may comprise at least one polyadenylation sequence. Polyadenylation is the addition of a poly(A) tail to a messenger RNA consisting of multiple adenosine monophosphates. In eukaryotes, polyadenylation is part of the process that produces mature messenger RNA (mRNA) for translation. The process of polyadenylation begins as the transcription of a gene terminates. The 3′-most segment of the newly made pre-mRNA is first cleaved off by a set of proteins; these proteins then synthesize the poly(A) tail at the RNA's 3′ end. The polyadenylation signal varies between groups of eukaryotes. Most human polyadenylation sites contain the AAUAAA sequence. Still further, in some alternative embodiments, the nucleic acid molecules, vectors and/or cassette provided by the invention and by the methods and compositions of the invention may comprise at least one enhancer. A transcription enhancer is a short (50-1500 bp) region of DNA that can be bound by proteins (activators) to increase the likelihood that transcription of a particular gene will occur. These proteins are usually referred to as transcription factors. Enhancers are generally cis-acting but can also be trans-acting (acting away from the gene) and can be located up to 1 Million bp (1,000,000 bp) away from the gene and can be upstream or downstream from the start site, and either in the forward or backward direction. There are hundreds of thousands of enhancers in the human genome. The invention thus encompasses in some embodiments thereof the use of any suitable enhancer. In some embodiments, the enhancer sequence may be located 3′ to the nucleic acid sequence encoding the CAR-Bs disclosed herein.

The nucleic acid molecules or any cassette/s thereof of the present disclosure enables targeted insertion of the nucleic acid sequences encoding the CAR/s of the invention into any specific or non-specific genomic sequence. Such target locus may reside according to some embodiments, within the IgH locus. It should be therefore appreciated that in some embodiments, the nucleic acid molecule/s of the invention or any cassette/s thereof used by the invention may further comprise targeting elements facilitating the specific recognition and targeted insertion or the CAR-B encoding sequences into the target site. Thus, according to some embodiments, the nucleic acid molecule of the invention may be flanked at the 5′ and/or 3′ thereof by at least one of (i) homology arms, for integration by homologous recombination; and (ii) recognition sites for a site-specific nuclease, a site-specific integrase or a site-specific recombinase. It should be appreciated that as described herein before, the homology arms used by the invention may be universal homology arms.

All elements required for successful targeting and recombination of the nucleic acid molecules, vectors and/or cassettes of the invention, are as defined in connection with other aspects of the invention.

The invention provides nucleic acid molecules, sequences encoding the CAR-Bs of the present disclosure, cassette, and methods, cells, uses and compositions thereof. The term “nucleic acid”, “nucleic acid sequence”, or “polynucleotide” and “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included. The terms should also be understood to include, as equivalents, analogs of either RNA or DNA made from nucleotide analogs, and, as applicable to the embodiment being described, single-stranded (such as sense or antisense) and double-stranded polynucleotides. Preparation of nucleic acids is well known in the art. Still further, it should be understood that the invention encompasses as additional aspects thereof any vector or vehicle that comprise any of the nucleic acid molecule/s of the invention or any cassettes described by the invention.

Still further, in some embodiments, the nucleic acid molecule/s of the invention or any cassette used by the invention may be comprised within a nucleic acid vector. In more specific embodiments, such vector may be any one of a viral vector, a non-viral vector and a naked DNA vector.

Vectors, as used herein, are nucleic acid molecules of particular sequence can be incorporated into a vehicle that is then introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Many vectors, e.g., plasmids, cosmids, minicircles, phage, viruses, etc., useful for transferring nucleic acids into target cells may be applicable in the present invention. The vectors comprising the nucleic acid(s) may be maintained episomally, e.g., as plasmids, minicircle DNAs, viruses such cytomegalovirus, adenovirus, etc., or they may be integrated into the target cell genome, through homologous recombination or random integration, e.g., retrovirus-derived vectors such as AAV, MMLV, HIV-1, ALV, etc. Vectors may be provided directly to the subject cells. In other words, the cells are contacted with vectors comprising the nucleic acid molecules, and/or cassettes of the invention that comprise the nucleic acid sequence encoding the CAR-Bs disclosed herein such that the vectors are taken up by the cells. Methods for contacting cells with nucleic acid vectors that are plasmids, such as electroporation, calcium chloride transfection, and lipofection, are well known in the art. DNA can be introduced as naked nucleic acid, as nucleic acid complexed with an agent such as a liposome or poloxamer, or can be delivered by viruses (e.g., adenovirus, AAV). More specifically, in some embodiments, the vector may be a viral vector. In yet some particular embodiments, such viral vector may be any one of recombinant adeno associated vectors (rAAV), single stranded AAV (ssAAV), self-complementary rAAV (scAAV), Simian vacuolating virus 40 (SV40) vector, Adenovirus vector, helper-dependent Adenoviral vector, retroviral vector and l'lentiviral vector. As indicated above, in some embodiments, viral vectors may be applicable in the present invention. The term “viral vector” refers to a replication competent or replication-deficient viral particle which are capable of transferring nucleic acid molecules into a host. The term “virus” refers to any of the obligate intracellular parasites having no protein-synthesizing or energy-generating mechanism. The viral genome may be RNA or DNA contained with a coated structure of protein of a lipid membrane. Examples of viruses useful in the practice of the present invention include baculoviridiae, parvoviridiae, picornoviridiae, herepesviridiae, poxviridiae, adenoviridiae, picotmaviridiae. The term recombinant virus includes chimeric (or even multimeric) viruses, i.e., vectors constructed using complementary coding sequences from more than one viral subtype.

In some embodiments, the nucleic acid molecules, and/or cassette of the invention may be comprised within an Adeno-associated virus (AAV). The term “adenovirus” is synonymous with the term “adenoviral vector”. AAV is a single-stranded DNA virus with a small (˜20 nm) protein capsule that belongs to the family of parvoviridae, and specifically refers to viruses of the genus adenoviridiae. The term adenoviridiae refers collectively to animal adenoviruses of the genus mastadenovirus including but not limited to human, bovine, ovine, equine, canine, porcine, murine and simian adenovirus subgenera. In particular, human adenoviruses includes the A-F subgenera as well as the individual serotypes thereof the individual serotypes and A-F subgenera including but not limited to human adenovirus types 1, 2, 3, 4, 4a, 5, 6, 7, 8, 9, 10, 11 (AdllA and Ad IIP), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91. Due to its inability to replicate in the absence of helpervirus coinfections (typically Adenovirus or Herpesvirus infections) AAV is often referred to as dependovirus. AAV infections produce only mild immune responses and are considered to be nonpathogenic, a fact that is also reflected by lowered biosafety level requirements for the work with recombinant AAVs (rAAV) compared to other popular viral vector systems. Due to its low immunogenicity and the absence of cytotoxic responses AAV-based expression systems offer the possibility to express nucleic acid sequences encoding the CAR-Bs disclosed herein for months in quiescent cells. Production systems for rAAV vectors typically consist of a DNA-based vector containing a transgene expression cassette, which is flanked by inverted terminal repeats. Construct sizes are limited to approximately 4.7-5.0 kb, which corresponds to the length of the wild-type AAV genome. rAAVs are produced in cell lines. The expression vector is co-transfected with a helper plasmid that mediates expression of the AAV rep genes which are important for virus replication and cap genes that encode the proteins forming the capsid. Recombinant adeno-associated viral vectors can transduce dividing and non-dividing cells, and different rAAV serotypes may transduce diverse cell types. These single-stranded DNA viral vectors have high transduction rates and have a unique property of stimulating endogenous Homologous Recombination without causing double strand DNA breaks in the host genome.

It should be appreciated that many intermediate steps of the wild-type infection cycle of AAV depend on specific interactions of the capsid proteins with the infected cell. These interactions are crucial determinants of efficient transduction and expression of nucleic acid molecules encoding the CAR-Bs disclosed herein when rAAV is used as gene delivery tool. Indeed, significant differences in transduction efficacy of various serotypes for particular tissues and cell types have been described. Thus, in some embodiments AAV serotype 6 may be suitable for the invention. In yet some further embodiments, AAV serotype 8 may be suitable for the methods of the invention. In some embodiments, the AAV serotype 6 may be encoded by the nucleic acid sequence as denoted by GenBank accession number AF028704.1. In some embodiments, the AAV serotype 8 useful in the present disclosure may be encoded by the nucleic acid sequence as denoted by GenBank accession number NC_006261.1.

It is believed that a rate-limiting step for the AAV-mediated expression of transgenes is the formation of double-stranded DNA. Recent reports demonstrated the usage of rAAV constructs with a self-complementing structure (scAAV) in which the two halves of the single-stranded AAV genome can form an intra-molecular double-strand. This approach reduces the effective genome size usable for gene delivery to about 2.3 kB but leads to significantly shortened onsets of expression in comparison with conventional single-stranded AAV expression constructs (ssAAV). Thus, in some embodiments, ssAAV may be applicable as a viral vector by the methods of the invention.

In yet some further embodiments, HDAd vectors may be suitable for the methods of the invention. The Helper-Dependent Adenoviral (HDAd) vectors HDAds have innovative features including the complete absence of viral coding sequences and the ability to mediate high level transgene expression with negligible chronic toxicity. HDAds are constructed by removing all viral sequences from the adenoviral vector genome except the packaging sequence and inverted terminal repeats, thereby eliminating the issue of residual viral gene expression associated with early generation adenoviral vectors. HDAds can mediate high efficiency transduction, do not integrate in the host genome, and have a large cloning capacity of up to 37 kb, which allows for the delivery of multiple transgenes or entire genomic loci, or large cis-acting elements to enhance or regulate tissue-specific transgene expression. One of the most attractive features of HDAd vectors is the long-term expression of the transgene. Still further, in some embodiments, SV40 may be used as a suitable vector by the methods of the invention. SV40 vectors (SV40) are vectors originating from modifications brought to Simian virus-40 an icosahedral papovavirus. Recombinant SV40 vectors are good candidates for gene transfer, as they display some unique features: SV40 is a well-known virus, non-replicative vectors are easy-to-make, and can be produced in titers of 10(12) IU/ml. They also efficiently transduce both resting and dividing cells, deliver persistent transgene expression to a wide range of cell types, and are non-immunogenic. Present disadvantages of rSV40 vectors for gene therapy are a small cloning capacity and the possible risks related to random integration of the viral genome into the host genome. In certain embodiments, an appropriate vector that may be used by the invention may be a retroviral vector. A retroviral vector consists of proviral sequences that can accommodate the nucleic acid molecule encoding the CAR-Bs disclosed herein, to allow incorporation of both into the target cells. The vector may also contain viral and cellular gene promoters, to enhance expression of the nucleic acid molecule encoding the CAR-Bs disclosed herein in the target cells. Retroviral vectors stably integrate into the dividing target cell genome so that the introduced gene is passed on and expressed in all daughter cells. They contain a reverse transcriptase that allows integration into the host genome. In yet some alternative embodiments, lentiviral vectors may be used in the present invention. Lentiviral vectors are derived from lentiviruses which are a subclass of Retroviruses. Commonly used retroviral vectors are “defective”, i.e., unable to produce viral proteins required for productive infection. Rather, replication of the vector requires growth in a packaging cell line. To generate viral particles comprising the nucleic acid molecules, vectors and/or cassette in accordance with the invention, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing the retroviral vectors comprising the nucleic acid molecules, vectors and/or cassette of the invention that contains the nucleic acids sequence encoding the CAR-B of the invention, into packaging cell lines and of collecting the viral particles that are generated by the packaging lines are well known in the art. Nonviral vectors, in accordance with the invention, refer to all the physical and chemical systems except viral systems and generally include either chemical methods, such as cationic liposomes and polymers, or physical methods, such as gene gun, electroporation, particle bombardment, ultrasound utilization, and magnetofection. Efficiency of this system is less than viral systems in gene transduction, but their cost-effectiveness, availability, and more importantly reduced induction of immune system and no limitation in size of transgenic DNA compared with viral system have made them attractive also for gene delivery.

For example, physical methods applied for in vitro and in vivo gene delivery are based on making transient penetration in cell membrane by mechanical, electrical, ultrasonic, hydrodynamic, or laser-based energy so that DNA entrance into the targeted cells is facilitated.

In more specific embodiments, the vector may be a naked DNA vector. More specifically, such vector may be for example, a plasmid, minicircle or linear DNA. Naked DNA alone may facilitate transfer of a gene (2-19 kb) into skin, thymus, cardiac muscle, and especially skeletal muscle and liver cells when directly injected. It enables also long-term expression. Although naked DNA injection is a safe and simple method, its efficiency for gene delivery is quite low.

Minicircles are modified plasmid in which a bacterial origin of replication (ori) was removed, and therefore they cannot replicate in bacteria. Linear DNA or Doggybone™ are double-stranded, linear DNA construct that solely encodes an antigen expression cassette, comprising antigen, promoter, polyA tail and telomeric ends. It should be appreciated that all DNA vectors disclosed herein, may be also applicable for all nucleic acid molecules, vectors and/or cassettes used in the methods and compositions of the invention, as described herein. Still further, it must be appreciated that the invention further provides any vectors or vehicles that comprise any of the nucleic acid molecules, vectors and/or nucleic acid cassettes disclosed by the invention, as well as any host cell expressing the nucleic acid molecules, and/or nucleic acid cassettes disclosed by the invention.

It should be further appreciated that the invention provides any CAR-B, any co-expressed BCR or antibody encoded by any of the nucleic acid molecule/s of the invention.

A further aspect of the present disclosure relates to a gene editing system comprising: (i) at least one nucleic acids molecule as defined by the present invention, or any cassette, vector or vehicle comprising said at least one nucleic acid molecule; and (ii) at least one gene editing component or a nucleic acid sequence encoding the gene editing component.

In some embodiments, a gene editing component may be any one of a site-specific nuclease, a class switch recombination, a site specific integrase, a site-specific recombinase and a recombination activating gene (RAG)-catalyzed recombination.

In some specific embodiments, a gene editing component useful in the systems of the present disclosure may be the CRISPR/Cas.

More specifically, in some embodiments, the CAR-B encoding nucleic acid sequences (e.g., in a nucleic acid cassette) is inserted into the appropriate genomic locus using a site-specific nuclease. The nuclease may be one of the following: CRISPR/Cas9/Cpf1/CTc(1/2/3), SpCas9, SaCas9, engineered CAS9, ZFN, TALEN, Homing endonuclease, Meganuclease, Mega-TALEN. The nuclease may be coded on a DNA vector such as a plasmid, a mini-circle or a viral vector. Alternatively, the mRNA coding for the nuclease may be delivered, or the nuclease may be delivered as a protein. A guide RNA may be provided or a DNA vector coding for a guide RNA. Integration catalyzed by a nuclease may utilize homologous arms flanking the DNA to be inserted or utilize recognition sites for the site-specific nuclease when such were coded preceding and or following the DNA to be inserted. Delivery of the nuclease or the vector coding for the nuclease can take place in vivo or ex vivo using autologous or allogeneic cells.

As specified above, in some embodiments, the nuclease used for targeted insertion of the nucleic acid sequence encoding the desired CAR molecule disclosed herein may comprise at least one component of the CRISPR-Cas system. The Clustered Regularly Interspaced Short Palindromic Repeats (CRISPR) system is a bacterial immune system that has been modified for genome engineering. CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI.

It should be understood that the invention contemplates the use of any of the known CRISPR systems, particularly and of the CRISPR systems disclosed herein. The CRISPR-Cas system has evolved in prokaryotes to protect against phage attack and undesired plasmid replication by targeting foreign DNA or RNA. In bacterial immunity, the CRISPR-Cas system, targets DNA molecules based on short homologous DNA sequences, called spacers that have previously been extracted by the bacterium from the foreign pathogen sequence and inserted between repeats as a memory system. These spacers are transcribed and processed and this RNA, named crRNA or guide-RNA (gRNA), guides CRISPR-associated (Cas) proteins to matching (and/or complementary) sequences within the foreign DNA, called proto-spacers, which are subsequently cleaved. The spacers, or other suitable constructs or RNAs can be rationally designed and produced to target any DNA sequence. Moreover, this recognition element may be designed separately to recognize and target any desired target including outside of a bacterium.

In some specific embodiment, the CRISPR-Cas proteins used in the present disclosure may be of a CRISPR Class 2 system. In yet some further particular embodiments, such class 2 system may be any one of CRISPR type II, and type V systems. In certain embodiments, the Cas applicable in the present invention may be any Cas protein of the CRISPR type II system. The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas1 and Cas2. Cas9 contains at least two nuclease domains, a RuvC-like nuclease domain near the amino terminus and the HNH (or MerA-like) nuclease domain in the middle of the protein. It should be appreciated that any type II CRISPR-Cas systems may be applicable in the present invention, specifically, any one of type II-A or B. Thus, in yet some further and alternative embodiments, at least one cas gene used in the methods and systems of the invention may be at least one cas gene of type II CRISPR system (either typeII-A or typeII-B). In more particular embodiments, at least one cas gene of type II CRISPR system used by the methods and systems of the invention may be the cas9 gene.

According to such embodiments, the CRISPR-Cas proteins used in the systems of the invention is a CRISPR-associated endonuclease 9 (Cas9). Double-stranded DNA (dsDNA) cleavage by Cas9 is a hallmark of “type II CRISPR-Cas” immune systems. The CRISPR-associated protein Cas9 is an RNA-guided DNA endonuclease that uses RNA:DNA complementarity to a target site (proto-spacer). After recognition between Cas9 and the target sequence double stranded DNA (dsDNA) cleavage occur, creating the double strand breaks (DSBs).

CRISPR type II system as used herein requires the inclusion of two essential components: a “guide” RNA (gRNA) and a CRISPR-associated endonuclease (Cas9). The gRNA is an RNA molecule composed of a “scaffold” sequence necessary for Cas9-binding (also named tracrRNA) and about 20 nucleotide long “spacer” or “targeting” sequence, which defines the genomic target to be modified. Guide RNA (gRNA), as used herein refers to a synthetic fusion or alternatively, annealing of the endogenous tracrRNA with a targeting sequence (also named crRNA), providing both scaffolding/binding ability for Cas9 nuclease and targeting specificity. Also referred to as “single guide RNA” or “sgRNA” or as a specificity conferring nucleic acid (SCNA).

In yet some further particular embodiments, the class 2 system in accordance with the invention, may be a CRISPR type V system. In a more specific embodiment, the RNA guided DNA binding protein nuclease may be CRISPR-associated endonuclease X (CasX) system or CRISPR-associated endonuclease 14 (Cas14) system or CRISPR-associated endonuclease F (CasF, also known as Cas12j) system. The type V CRISPR-Cas systems are distinguished by a single RNA-guided RuvC domain-containing nuclease. As with type II CRISPR-Cas systems, CRISPR type V system as used herein requires the inclusion of two essential components: a gRNA and a CRISPR-associated endonuclease (CasX/Cas14/CasF). The gRNA is a short synthetic RNA composed of a “scaffold” sequence necessary for CasX/Cas14/CasF-binding and about 20 nucleotide long “spacer” or “targeting” sequence, which defines the genomic target to be modified.

It should be noted that the gRNA used herein may comprise between about 3 nucleotides to about 100 nucleotides, specifically, 3, 4, 5, 6, 7, 8, 9, 10, 15, 20, 25, 30, 35, 40, 45, 50, 55, 60, 65, 70, 75, 80, 85, 90, 95 100 or more. More specifically between about 10 nucleotides to 70 nucleotides or more.

It should be noted that any CRISPR/Cas proteins may be used by the invention, in some embodiments of the present disclosure, the endonuclease may be a Cas9, CasX, Cas12, Cas13, Cas14, Cas6, Cpf1, CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R, Corynebacterium kroppenstedtii (DSM 44385), Mycobacterium abscessus (ATCC 19977), Nocardia farcinica IFM10152, Rhodococcus erythropolis PR4, Rhodococcus jostii RFIA1, Rhodococcus opacus B4 (uid36573), Acidothermus cellulolyticus 11 B, Arthrobacter chlorophenolicus A6, Kribbella flavida (DSM 17836), Thermomonospora curvata (DSM43183), Bifidobacterium dentium Bd1, Bifidobacterium longum DJO10A, Slackia heliotrinireducens (DSM 20476), Persephonella marina EX H 1, Bacteroides fragilis NCTC 9434, Capnocytophaga ochracea (DSM 7271), Flavobacterium psychrophilum JIP02 86, Akkermansia muciniphila (ATCC BAA 835), Roseiflexus castenholzii (DSM 13941), Roseiflexus RS1, Synechocystis PCC6803, Elusimicrobium minutum Pei191, uncultured Termite group 1 bacterium phylotype Rs D17, Fibrobacter succinogenes S85, Bacillus cereus (ATCC 10987), Listeria innocua, Lactobacillus casei, Lactobacillus rhamnosus GG, Lactobacillus salivarius UCC118, Streptococcus agalactiae-5-A909, Streptococcus agalactiae NEM316, Streptococcus agalactiae 2603, Streptococcus dysgalactiae equisimilis GGS 124, Streptococcus equi zooepidemicus MGCS10565, Streptococcus gallolyticus UCN34 (uid46061), Streptococcus gordonii Challis subst CH1, Streptococcus mutans NN2025 (uid46353), Streptococcus mutans, Streptococcus pyogenes M1 GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS6180, Streptococcus pyogenes MGAS315, Streptococcus pyogenes SSI-1, Streptococcus pyogenes MGAS10750, Streptococcus pyogenes NZ131, Streptococcus thermophiles CNRZ1066, Streptococcus thermophiles LMD-9, Streptococcus thermophiles LMG 18311, Clostridium botulinum A3 Loch Maree, Clostridium botulinum B Eklund 17B, Clostridium botulinum Ba4 657, Clostridium botulinum F Langeland, Clostridium cellulolyticum H10, Finegoldia magna (ATCC 29328), Eubacterium rectale (ATCC 33656), Mycoplasma gallisepticum, Mycoplasma mobile 163K, Mycoplasma penetrans, Mycoplasma synoviae 53, Streptobacillus, moniliformis (DSM 12112), Bradyrhizobium BTAil, Nitrobacter hamburgensis X14, Rhodopseudomonas palustris BisB18, Rhodopseudomonas palustris BisB5, Parvibaculum lavamentivorans DS-1, Dinoroseobacter shibae. DFL 12, Gluconacetobacter diazotrophicus Pal 5 FAPERJ, Gluconacetobacter diazotrophicus Pal 5 JGI, Azospirillum B510 (uid46085), Rhodospirillum rubrum (ATCC 11170), Diaphorobacter TPSY (uid29975), Verminephrobacter eiseniae EF01-2, Neisseria meningitides 053442, Neisseria meningitides alpha14, Neisseria meningitides Z2491, Desulfovibrio salexigens DSM 2638, Campylobacter jejuni doylei 269 97, Campylobacter jejuni 81116, Campylobacter jejuni, Campylobacter lari RM2100, Helicobacter hepaticus, Wolinella succinogenes, Tolumonas auensis DSM 9187, Pseudoalteromonas atlantica T6c, Shewanella pealeana (ATCC 700345), Legionella pneumophila Paris, Actinobacillus succinogenes 130Z, Pasteurella multocida, Francisella tularensis novicida U 112, Francisella tularensis holarctica, Francisella tularensis FSC 198, Francisella tularensis, Francisella tularensis WY96-3418, or Treponema denticola (ATCC 35405).

In some embodiments, the CAR-B encoding nucleic acid sequences (e.g. in a nucleic acid cassette) is inserted into the appropriate genomic locus using a site specific recombinase/integrase. The recombinase/integrase may be one of the following: PhiC31, HK022, Cre, Flp, and more. The recombinase/integrase may be coded on a DNA vector such as a plasmid, a mini-circle or a viral vector. Alternatively, the mRNA coding for the recombinase/integrase may be delivered, or the recombinase/integrase may be delivered as a protein. Delivery of the nuclease or the vector coding for the recombinase/integrase can take place in vivo or ex vivo using autologous or allogeneic cells.

In some embodiments, the CAR-B encoding nucleic acid sequences (e.g., in a nucleic acid cassette) is inserted into the appropriate genomic locus using by utilizing the CSR process. The DNA to be inserted or the vector coding for the DNA to be inserted may be delivered to cells undergoing CSR, or to cells about to undergo CSR or to cells that can be induced to undergo CSR.

In some embodiments, the CAR-B encoding nucleic acid sequences (e.g., in a nucleic acid cassette) is inserted into the appropriate genomic locus by utilizing the VDJ recombination process. The DNA to be inserted or the vector coding for the DNA to be inserted may be delivered to cells undergoing VDJ recombination, or to cells about to undergo VDJ recombination or to cells that can be induced to undergo VDJ recombination.

It should be understood that the disclosed nucleic acid sequences encoding the disclosed CAR-B molecules of the invention, and optionally any further effector proteins, may be inserted into any appropriate genomic locus. In some particular and non-limiting embodiments, the CAR encoding nucleic acid sequences may be inserted into a target genomic sequence within the Immunoglobulin heavy chain (IgH) locus. In more specific embodiments, such nucleic acid molecules may be inserted downstream to the J region of the variable domain and upstream of at least one splice acceptor site of the constant domain of said heavy chain of a B cell receptor (BCR) of said cell of the B lineage. In yet some more particular embodiments, the target genomic sequence within the IgH locus is located downstream to the J region of the variable domain and upstream of the class switch recombination (CSR) region of said heavy chain.

The term “host cell” includes a cell into which a heterologous (e.g., exogenous) nucleic acid or protein has been introduced. Persons of skill upon reading this disclosure will understand that such terms refer not only to the particular subject cell, but also is used to refer to the progeny of such a cell. Because certain modifications may occur in succeeding generations due to either mutation or environmental influences, such progeny may not, in fact, be identical to the parent cell, but are still included within the scope of the term “host cell”. As used herein, a cell has been “transformed” or “transfected” by exogenous or heterologous DNA, e.g., the nucleic acid molecule/s of the invention or any cassette of the invention, when such DNA has been introduced inside the cell. The transforming DNA may be integrated (covalently linked) into the genome of the cell. With respect to the present invention, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. It should be appreciated that in some embodiments, the host cells of the invention may be any engineered B cells of the invention or any cell population comprising, at least in part, the B cells of the invention. Still further, the invention further encompasses any population of cells comprising at least 5%, 10%, 15%, 20%, 25%, 30%, 35%, 40%, 45%, 50%, 55%, 60%, 65%, 70%, 75%, 80%, 85%, 90%, 95%, 99.9% or more, specifically, 100%) specifically, 10%, 15%, 20%, 25%, 30%, 35%, 40%, 45%, 50%, 55%, 60%, 65%, 70%, 75%, 80%, 85%, 90%, 95%, 99.9% or more, specifically, 100% of the host cells of the invention.

A further aspect of the present disclosure relates to a pharmaceutical composition comprising at least one CAR molecule that allows a cell of the B cell lineage to undergo antigen-induced activation independent of a cell of the T cell lineage, or any system comprising the CAR molecule disclosed herein, any nucleic acid molecule comprising at least one nucleic acid sequence encoding the CAR molecule, or any, cassette, vector, vehicle or gene editing system comprising the nucleic acid molecule, any cell expressing the CAR molecule of the present disclosure, or any genetically engineered B cell expressing the CAR molecule or population of cells comprising at least one the genetically engineered B cell disclosed herein. In some embodiments, the CAR molecule comprises the following components: (i) at least one target-binding domain; (ii) at least one transmembrane domain; and (iii) at least one signal transduction domain. It should be understood that this signal transduction domain is capable of relaying, and transducing signals for T cell independent activation. In some optional embodiments, the composition disclosed herein may further comprise at least one of pharmaceutically acceptable carrier/s, diluent/s, excipient/s and additive/s. As indicated above, in some optional embodiments, the compositions of the invention may further comprise at least one of pharmaceutically acceptable carrier/s, diluent/s, excipient/s and additive/s.

The compositions of the invention may comprise an effective amount of the nucleic acid molecules, and/or cassette thereof or of any vector thereof or of any cell comprising the same, or any BCR as described by the invention, or any antibody derived therefrom. The term “effective amount” relates to the amount of an active agent present in a composition, specifically, the nucleic acid molecules, vectors and/or cassette of the invention as described herein that is needed to provide a desired level of active agent in the bloodstream or at the site of action in an individual (e.g., the thymus or bone marrow) to be treated to give an anticipated physiological response when such composition is administered. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein. An “effective amount” of the nucleic acid molecule/s of the invention or any cassette of the invention can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

The pharmaceutical compositions of the invention can be administered and dosed by the methods of the invention, in accordance with good medical practice, systemically, for example by parenteral, e.g., intrathymic, into the bone marrow and intravenous. It should be noted however that the invention may further encompass additional administration modes. In other examples, the pharmaceutical composition can be introduced to a site by any suitable route including intraperitoneal, subcutaneous, transcutaneous, topical, intramuscular, intraarticular, subconjunctival, or mucosal, e.g., oral, intranasal, or intraocular administration.

Local administration to the area in need of treatment may be achieved by, for example, by local infusion during surgery, topical application, direct injection into the specific organ (bone marrow, spleen, lymph nodes), etc. More specifically, the compositions used in any of the methods of the invention, described herein, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically, the CAR-B, nucleic acid molecule/s of the invention or any cassette/s thereof, any co-expressed BCR, cells and systems of the invention with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations. It should be understood that in addition to the ingredients particularly mentioned above, the formulations may also include other agents conventional in the art having regard to the type of formulation in question. Still further, pharmaceutical preparations are compositions that include one or more nucleic acid molecules, vectors and/or cassette present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle”, when referred to the compositions in the present aspect, refers to a diluent, adjuvant, excipient, or carrier with which a compound of the invention is formulated for administration to a mammal. Such pharmaceutical vehicles can be lipids, e.g., liposomes, e.g., liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the nucleic acid molecule/s encoding the CARs of the invention or any co-expressed BCRs or any other effector cells and systems of the invention, can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. The active agent may be formulated for immediate activity, or it may be formulated for sustained release.

The present disclosure provides method allowing in vivo as well as ex-vivo or in vitro genetic engineering of cells of the B lineage to express the CAR-B molecules of the present invention. In case the engineering of the cells is performed ex vivo or in vitro, the engineered cells are transferred back to the subject, by adoptive transfer.

The term “adoptive transfer” as herein defined applies to all the therapies that consist of the transfer of components of the immune system, specifically cells that are already capable of mounting a specific immune response. In such option, the targeted insertion of the nucleic acid sequence encoding the CAR-Bs disclosed herein, is performed in cells of an autologous or allogeneic source, that are then administered to the subject, specifically, by adoptive transfer.

In some embodiments, the cells that express, comprise, transduced or transfected with the nucleic acid molecule/s of the invention or any cassette provided by the invention may be cells of an autologous source. The term “autologous” when relating to the source of cells, refers to cells derived or transferred from the same subject that is to be treated by the method of the invention. The term “allogenic” when relating to the source of cells, refers to cells derived or transferred from a different subject, referred to herein as a donor, of the same species.

The present disclosure provide method applicable for various immune-related disorders. An “Immune-related disorder” or “Immune-mediated disorder”, as used herein encompasses any condition that is associated with the immune system of a subject, more specifically through inhibition of the immune system, or that can be treated, prevented or ameliorated by reducing degradation of a certain component of the immune response in a subject, such as the adaptive or innate immune response. An immune-related disorder may include infectious condition (e.g., by a pathogen, specifically, viral, bacterial or fungal infections), inflammatory disease, autoimmune disorders, metabolic disorders and proliferative disorders, specifically, cancer. In some specific embodiments wherein the immune-related disorder or condition may be a primary or a secondary immunodeficiency. ‘Innate immunity’ refers to immune responses found in all classes of plants and animals that provide immediate defense against pathogens, and also immune responses that are triggered at sites of infection.

‘Adaptive immunity’ refers to responses of the vertebrate immune system that provide specific and long-lasting protection against a particular antigen, also referred to as immunological memory, in peripheral lymphoid organs. As innate and adaptive immunity are interrelated, certain types of lymphocytes partake in both these systems.

In some specific embodiments, the methods of the invention may be used for treating proliferative disorders. As used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods of the present invention may be applicable for treatment of a patient suffering from any one of non-solid and solid tumors. Malignancy, as contemplated in the present invention may be any one of carcinomas, melanomas, lymphomas, leukemias, myeloma and sarcomas. Carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges. Melanoma as used herein, is a malignant tumor of melanocytes. Melanocytes are cells that produce the dark pigment, melanin, which is responsible for the color of skin. They predominantly occur in skin but are also found in other parts of the body, including the bowel and the eye. Melanoma can occur in any part of the body that contains melanocytes. Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Further malignancies that may find utility in the present invention can comprise but are not limited to hematological malignancies (including lymphoma, leukemia and myeloproliferative disorders, as described above), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The invention may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma. It should be appreciated that for treating cancer, the nucleic acid molecule/s of the invention that encode the CAR-B molecules disclosed herein, or any cassette/s thereof of the invention or any compositions or methods thereof may facilitate targeted insertion or antibody or receptor as described herein before, that are specifically directed at TAAs or any viral antigens.

It should be understood that the invention thus encompasses the treatment of any of the malignancies described in this context, specifically any malignancies described in connection with associated TAAs as described herein before in connection with other aspects of the invention. In yet some further embodiments, and of particular relevance are patients' populations diagnosed with one of autoimmune disorders, also referred to as disorders of immune tolerance, when the immune system fails to properly distinguish between self and non-self-antigens.

Thus, according to some embodiments, the method of the invention may be used for the treatment of a patient suffering from any autoimmune disorder. In some specific embodiments, the methods of the invention may be used for treating an autoimmune disease such as for example, but not limited to, inflammatory bowel disease (IBD), ulcerative colitis, Crohn's disease, fatty liver disease, Lymphocytic colitis, Ischaemic colitis, Diversion colitis, Behçet's syndrome, Indeterminate colitis, rheumatoid arthritis, systemic lupus erythematosus (SLE), Graft versus Host Disease (GvHD), Eaton-Lambert syndrome, Goodpasture's syndrome, Greave's disease, Guillain-Barr syndrome, autoimmune hemolytic anemia (AIHA), hepatitis, insulin-dependent diabetes mellitus (IDDM) and NIDDM, multiple sclerosis (MS), myasthenia gravis, plexus disorders e.g. acute brachial neuritis, polyglandular deficiency syndrome, primary biliary cirrhosis, scleroderma, thrombocytopenia, thyroiditis e.g. Hashimoto's disease, Sjogren's syndrome, allergic purpura, psoriasis, mixed connective tissue disease, polymyositis, dermatomyositis, vasculitis, polyarteritis nodosa, arthritis, alopecia areata, polymyalgia rheumatica, Wegener's granulomatosis, Reiter's syndrome, ankylosing spondylitis, pemphigus, bullous pemphigoid, dermatitis herpetiformis, psoriatic arthritis, reactive arthritis, and ankylosing spondylitis, inflammatory arthritis, including juvenile idiopathic arthritis, gout and pseudo gout, as well as arthritis associated with colitis or psoriasis, Pernicious anemia, some types of myopathy and Lyme disease (Late).

In yet some other embodiments, the methods of the invention may be also applicable for treating a subject suffering from an infectious disease. More specifically, such infectious disease may be any pathological disorder caused by a pathogen. As used herein, the term “pathogen” refers to an infectious agent that causes a disease in a subject host. Pathogenic agents include prokaryotic microorganisms, lower eukaryotic microorganisms, complex eukaryotic organisms, viruses, fungi, mycoplasma, prions, parasites, for example, a parasitic protozoan, yeasts or a nematode.

In yet some further embodiments, the methods of the invention may be applicable in boosting the immune response against a pathogen that may be in further specific embodiment, a viral pathogen or a virus. The term “virus” as used herein, refers to obligate intracellular parasites of living but non-cellular nature, consisting of DNA or RNA and a protein coat. Viruses range in diameter from about 20 to about 300 nm. Class I viruses (Baltimore classification) have a double-stranded DNA as their genome; Class II viruses have a single-stranded DNA as their genome; Class III viruses have a double-stranded RNA as their genome; Class IV viruses have a positive single-stranded RNA as their genome, the genome itself acting as mRNA; Class V viruses have a negative single-stranded RNA as their genome used as a template for mRNA synthesis; and Class VI viruses have a positive single-stranded RNA genome but with a DNA intermediate not only in replication but also in mRNA synthesis. It should be noted that the term “viruses” is used in its broadest sense to include viruses of the families adenoviruses, papovaviruses, herpesviruses: simplex, varicella-zoster, Epstein-Barr (EBV), Cytomegalo virus (CMV), pox viruses: smallpox, vaccinia, hepatitis B (HBV), rhinoviruses, hepatitis A (HBA), poliovirus, respiratory syncytial virus (RSV), Middle East Respiratory Syndrome (MERS), Severe acute respiratory syndrome (SARS), rubella virus, hepatitis C (HBC), arboviruses, rabies virus, influenza viruses A and B, measles virus, mumps virus, human deficiency virus (HIV), HTLV I and II, Dengue virus and Zika virus.

In some further embodiments, the methods of the invention may be applicable for immune-related disorder or condition that may be a pathologic condition caused by at least one pathogen. It should be appreciated that an infectious disease as used herein also encompasses any infectious disease caused by a pathogenic agent, specifically, a pathogen. Pathogenic agents include prokaryotic microorganisms, lower eukaryotic microorganisms, complex eukaryotic organisms, viruses, fungi, prions, parasites, yeasts, toxins and venoms. In yet some other specific embodiments, the methods and composition of the invention may be applicable for treating an infectious disease caused by bacterial pathogens. More specifically, a prokaryotic microorganism includes bacteria such as Gram positive, Gram negative and Gram variable bacteria and intracellular bacteria. Examples of bacteria contemplated herein include the species of the genera Treponema sp., Borrelia sp., Neisseria sp., Legionella sp., Bordetella sp., Escherichia sp., Salmonella sp., Shigella sp., Klebsiella sp., Yersinia sp., Vibrio sp., Hemophilus sp., Rickettsia sp., Chlamydia sp., Mycoplasma sp., Staphylococcus sp., Streptococcus sp., Bacillus sp., Clostridium sp., Corynebacterium sp., Proprionibacterium sp., Mycobacterium sp., Ureaplasma sp. and Listeria sp.

Particular species include Treponema pallidum, Borrelia burgdorferi, Neisseria gonorrhea, Neisseria meningitidis, Legionella pneumophila, Bordetella pertussis, Escherichia coli, Salmonella typhi, Salmonella typhimurium, Shigella dysenteriae, Klebsiella pneumoniae, Yersinia pestis, Vibrio cholerae, Hemophilus influenzae, Rickettsia rickettsii, Chlamydia trachomatis, Mycoplasma pneumoniae, Staphylococcus aureus, Streptococcus pneumoniae, Streptococcus pyogenes, Bacillus anthracis, Clostridium botulinum, Clostridium tetani, Clostridium perfringens, Corynebacterium diphtheriae, Proprionibacterium acnes, Mycobacterium tuberculosis, Mycobacterium leprae and Listeria monocytogenes. A lower eukaryotic organism includes a yeast or fungus such as but not limited to Pneumocystis carinii, Candida albicans, Aspergillus, Histoplasma capsulatum, Blastomyces dermatitidis, Cryptococcus neoformans, Trichophyton and Microsporum, are also encompassed by the invention. A complex eukaryotic organism includes worms, insects, arachnids, nematodes, aemobe, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania. More specifically, in certain embodiments the methods and compositions of the invention may be suitable for treating disorders caused by fungal pathogens. The term “fungi” (or a “fungus”), as used herein, refers to a division of eukaryotic organisms that grow in irregular masses, without roots, stems, or leaves, and are devoid of chlorophyll or other pigments capable of photosynthesis. Each organism (thallus) is unicellular to filamentous and possess branched somatic structures (hyphae) surrounded by cell walls containing glucan or chitin or both, and containing true nuclei. It should be noted that “fungi” includes for example, fungi that cause diseases such as ringworm, histoplasmosis, blastomycosis, aspergillosis, cryptococcosis, sporotrichosis, coccidioidomycosis, paracoccidio-idoinycosis, and candidiasis.

As noted above, the present invention also provides for the methods and compositions for the treatment of a pathological disorder caused by “parasitic protozoan”, which refers to organisms formerly classified in the Kingdom “protozoa”. They include organisms classified in Amoebozoa, Excavata and Chromalveolata. Examples include Entamoeba histolytica, Plasmodium (some of which cause malaria), and Giardia lamblia. The term parasite includes, but not limited to, infections caused by somatic tapeworms, blood flukes, tissue roundworms, ameba, and Plasmodium, Trypanosoma, Leishmania, and Toxoplasma species. As used herein, the term “nematode” refers to roundworms. Roundworms have tubular digestive systems with openings at both ends. Some examples of nematodes include, but are not limited to, basal order Monhysterida, the classes Dorylaimea, Enoplea and Secernentea and the “Chromadorea” assemblage.

In yet some further specific embodiments, the present invention provides compositions and methods for use in the treatment, prevention, amelioration or delay the onset of a pathological disorder, wherein said pathological disorder is a result of a prion. As used herein, the term “prion” refers to an infectious agent composed of protein in a misfolded form. Prions are responsible for the transmissible spongiform encephalopathies in a variety of mammals, including bovine spongiform encephalopathy (BSE, also known as “mad cow disease”) in cattle and Creutzfeldt-Jakob disease (CJD) in humans. All known prion diseases affect the structure of the brain or other neural tissue and all are currently untreatable and universally fatal. It should be appreciated that an infectious disease as used herein also encompasses any pathologic condition caused by toxins and venoms.

Still further, the methods of the invention may offer a promising therapeutic modality for a variety of innate and acquired immunodeficiencies caused by immunosuppressive treatments (chemo- and radiotherapy), pathogenic infections, cancer and HSCT. More specifically, Immunodeficiency (or immune deficiency) is a state in which the immune system's ability to fight infectious disease and cancer is compromised or entirely absent. Most cases of immunodeficiency are acquired (“secondary”) due to extrinsic factors that affect the patient's immune system. Examples of these extrinsic factors include viral infection, specifically, HIV, extremes of age, and environmental factors, such as nutrition. In the clinical setting, the immunosuppression by some drugs, such as steroids, can be either an adverse effect or the intended purpose of the treatment. Examples of such use are in organ transplant surgery as an anti-rejection measure and in patients suffering from an overactive immune system, as in autoimmune diseases. Immunodeficiency also decreases cancer immuno-surveillance, in which the immune system scans the cells and kills neoplastic ones. Still further, Primary immunodeficiencies (PID), also termed innate immunodeficiencies, are disorders in which part of the organism immune system is missing or does not function normally. To be considered a primary immunodeficiency, the cause of the immune deficiency must not be caused by other disease, drug treatment, or environmental exposure to toxins). Most primary immune-deficiencies are genetic disorders; the majority is diagnosed in children under the age of one, although milder forms may not be recognized until adulthood. While there are over 100 recognized PIDs, most are very rare. There are several types of immunodeficiency that include, Humoral immune deficiency (including B cell deficiency or dysfunction), which generally includes symptoms of hypogammaglobulinemia (decrease of one or more types of antibodies) with presentations including repeated mild respiratory infections, and/or agammaglobulinemia (lack of all or most antibody production) and results in frequent severe infections (mostly fatal); T cell deficiency, often causes secondary disorders such as acquired immune deficiency syndrome (AIDS); Granulocyte deficiency, including decreased numbers of granulocytes (called as granulocytopenia or, if absent, agranulocytosis) such as of neutrophil granulocytes (termed neutropenia); granulocyte deficiencies also include decreased function of individual granulocytes, such as in chronic granulomatous disease; Asplenia, where there is no function of the spleen; and Complement deficiency in which the function of the complement system is deficient. Secondary immunodeficiencies occur when the immune system is compromised due to environmental factors. Such factors include but are not limited to radiotherapy as well as chemotherapy. While often used as fundamental anti-cancer treatments, these modalities are known to suppress immune function, leaving patients with an increased risk of infection; indeed, infections were found to be a leading cause of patient death during cancer treatment. Neutropenia was specifically associated with vulnerability to life-threatening infections following chemotherapy and radiotherapy. In more specific embodiments, such secondary immunodeficiency may be caused by at least one of chemotherapy, radiotherapy, biological therapy, bone marrow transplantation, gene therapy, adoptive cell transfer or any combinations thereof.

More specifically, according to the International Union of Immunological Societies, more than 150 primary immunodeficiency diseases (PIDs) have been characterized, and the number of acquired (or secondary) immuno-deficiencies exceeds the number of PIDs. PIDs are those caused by inherited genetic mutations. Secondary immuno-deficiencies are caused by various conditions, aging or agents such as viruses or immune suppressing drugs. A number of notable examples of PIDs include Severe combined immunodeficiency (SCID), DiGeorge syndrome, Hyperimmunoglobulin E syndrome (also known as Job's Syndrome), Common variable immunodeficiency (CVID): B-cell levels are normal in circulation but with decreased production of IgG throughout the years, so it is the only primary immune disorder that presents onset in the late teens. Chronic granulomatous disease (CGD): a deficiency in NADPH oxidase enzyme, which causes failure to generate oxygen radicals. Classical recurrent infection from catalase positive bacteria and fungi. Wiskott-Aldrich syndrome (WAS); autoimmune lymphoproliferative syndrome (ALPS); Hyper IgM syndrome: X-linked disorder that causes a deficiency in the production of CD40 ligand on activated T-cells. This increases the production and release of IgM into circulation. The B-cell and T-cell numbers are within normal limits. Increased susceptibility to extracellular bacteria and opportunistic infections. Leukocyte adhesion deficiency (LAD); NF-κB Essential Modifier (NEMO) Mutations; Selective immunoglobulin A deficiency: the most common defect of the humoral immunity, characterized by a deficiency of IgA. Produces repeating sino-pulmonary and gastrointestinal infections. X-linked agammaglobulinemia (XLA; also known as Bruton type agammaglobulinemia): characterized by a deficiency in tyrosine kinase enzyme that blocks B-cell maturation in the bone marrow. No B-cells are produced to circulation and thus, there are no immunoglobulin classes, although there tends to be a normal cell-mediated immunity. X-linked lymphoproliferative disease (XLP); and Ataxia-telangiectasia.

Thus, patients' populations diagnosed with one of PIDs can particularly benefit from methods and compositions of modulators according to the present invention.

With respect to secondary immunodeficiencies, those can be manifested in both the young and the elderly. Under normal conditions immune responses are beginning to decline at around 50 years of age, what is called immunosenescence. The term ‘immunosenescence’ refers to the gradual deterioration of the immune system brought on by natural age advancement. It involves both the host's capacity to respond to infections and the development of long-term immune memory. Additional common causes of secondary immunodeficiency include severe burns, malnutrition, certain types of cancer, and chemotherapy in cancer patients.

More specifically, in developed countries, obesity, alcoholism, and drug use are common causes of poor immune function. However, malnutrition is the most common cause of immunodeficiency in developing countries. Diets lacking sufficient protein are associated with impaired cell-mediated immunity, complement activity, phagocyte function, IgA antibody concentrations, and cytokine production. Additionally, the loss of the thymus at an early age through surgical removal, for example, results in severe immunodeficiency and high susceptibility to infections.

Of particular relevance to the present context are cellular immunodeficiencies associated with cancer and certain viral pathogens. A cellular immunodeficiency refers to a deficiency the count or function of T lymphocytes, which are the main type of cells responsible for the cellular adaptive immune response in attacking viruses, cancer cells and other parasites. Extensive research has reasonably well established the role of immunodeficiency in cancers of the head and neck, lung, esophagus and breast. Among virally induced immunodeficiencies, the most notable example is AIDS (Acquired Immunodeficiency Syndrome) cause by HIV infection. The role of HIV as a direct cause of cellular immunodeficiency, particularly the deficiency of the CD4+T helper lymphocyte population, has been well established. Additional examples of viral- or pathogen-induced immunodeficiencies include, although not limited to chickenpox, cytomegalovirus, German measles, measles, tuberculosis, infectious mononucleosis (Epstein-Barr virus), chronic hepatitis, lupus, and bacterial and fungal infections. One of the most recent examples is virus-induced Severe Acute Respiratory Syndrome (SARS). These and additional examples of disorders related to cellular immunodeficiency may include Aplastic anemia, Leukemia, Multiple myeloma, Sickle cell disease, chromosomal disorders such as Down syndrome, infectious diseases caused by pathogens such as Cytomegalovirus, Epstein-Barr virus, Human immunodeficiency virus (HIV), Measles and certain bacterial infections. Chronic kidney disease, Nephrotic syndrome, Hepatitis, Liver failure and other conditions caused by Malnutrition, alcoholism and burns.

Thus, patients' populations diagnosed with one of the secondary immunodeficiencies, and particularly one of the cellular immunodeficiencies as above, can particularly benefit from methods and compositions of modulators according to the present invention. Differential diagnosis of such immunodeficient patients is routinely performed in various clinical settings.

Additional secondary immunodeficiencies may result following bone marrow (BM) transplantation, gene therapy or adaptive cell transfer.

Hematopoietic stem cell transplantation (HSCT) is the transplantation of multipotent hematopoietic stem cells, usually derived from bone marrow, peripheral blood, or umbilical cord blood. It may be autologous (the patient's own stem cells are used), allogeneic (the stem cells come from a donor) or syngeneic (from an identical twin). Performance of this medical procedure usually requires the destruction of the recipient's immune system using radiation or chemotherapy before the transplantation. To limit the risks of transplanted stem cell rejection or of severe graft-versus-host disease in allogeneic HSCT, the donor should preferably have the same human leukocyte antigens (HLA) as the recipient. In the case of a bone marrow transplant, the HSC are removed from a large bone of the donor, typically the pelvis, through a large needle that reaches the center of the bone. Peripheral blood stem cells are now the most common source of stem cells for HSCT. They are collected from the blood through a process known as apheresis. The donor's blood is withdrawn through a sterile needle in one arm and passed through a machine that removes white blood cells. The red blood cells are returned to the donor. The peripheral stem cell yield is boosted with daily subcutaneous injections of Granulocyte-colony stimulating factor (G-CSF), serving to mobilize stem cells from the donor's bone marrow into the peripheral circulation. It should be noted that amniotic fluid as well as umbilical cord blood may be also used as a source of stem cells for HSCT.

“Protein misfolding and aggregation” as used herein, relates to an impaired physical process by which a protein chain acquires its native three-dimensional structure, a conformation that is usually biologically functional, in an expeditious and reproducible manner. It is the physical process by which a polypeptide folds into its characteristic and functional three-dimensional structure from random coil. Each protein exists as an unfolded polypeptide or random coil when translated from a sequence of mRNA to a linear chain of amino acids. Amino acids interact with each other to produce a well-defined three-dimensional structure, the folded protein, known as the native state. The correct three-dimensional structure is essential to function, although some parts of functional proteins may remain unfolded. Failure to fold into native structure generally produces inactive proteins, but in some instances misfolded proteins have modified or toxic functionality. Several neurodegenerative and other diseases are believed to result from the accumulation of amyloid fibrils formed by the association of misfolded proteins.

More specifically, under some conditions, proteins may not fold into their biochemically functional forms resulting in protein denaturation. A fully denatured protein lacks both tertiary and secondary structure and exists as a so-called random coil. Under certain conditions some proteins can refold; however, in many cases, denaturation is irreversible. Cells sometimes protect their proteins against the denaturing influence of heat with enzymes known as chaperones or heat shock proteins, which assist other proteins both in folding and in remaining folded. Some proteins never fold in cells at all except with the assistance of chaperone molecules, which either isolate individual proteins so that their folding is not interrupted by interactions with other proteins or help to unfold misfolded proteins, giving them a second chance to refold properly. This function is crucial to prevent the risk of precipitation into insoluble amorphous aggregates.

Aggregated proteins are associated with prion-related illnesses such as Creutzfeldt-Jakob disease, bovine spongiform encephalopathy (mad cow disease), amyloid-related illnesses such as Alzheimer's disease and familial amyloid cardiomyopathy or polyneuropathy, as well as intracytoplasmic aggregation diseases such as Huntington's and Parkinson's disease. These age onset degenerative diseases are associated with the aggregation of misfolded proteins into insoluble, extracellular aggregates and/or intracellular inclusions including cross-beta sheet amyloid fibrils. It is not completely clear whether the aggregates are the cause or merely a reflection of the loss of protein homeostasis, the balance between synthesis, folding, aggregation and protein turnover. Misfolding and excessive degradation instead of folding and function leads to a number of proteopathy diseases such as antitrypsin-associated emphysema, cystic fibrosis and the lysosomal storage diseases, where loss of function is the origin of the disorder.

As some of the conditions associated with protein misfolding and protein aggregations involve neurodegeneration, in certain specific embodiments, the invention may be applicable for neurodegenerative diseases.

The term “neurodegenerative diseases” is the general term for the progressive loss of structure or function of neurons, leading to their death. The greatest risk factor for neurodegenerative diseases is aging. Mitochondrial DNA mutations as well as oxidative stress both contribute to aging. Many of these diseases are late-onset, meaning there is some factor that change as a person ages, for each disease. One constant factor is that in each disease, neurons gradually lose function as the disease progresses with age.

Still further, it should be appreciated that in certain embodiments, the invention may be further applicable for disorders characterized by beta-amyloid protein aggregation.

A group of disorders associated with beta-amyloid protein aggregation include Alzheimer's disease (AD), where deposits of a protein precursor called beta-amyloid build up (termed plaques) in the spaces between nerve cells and twisted fibers of tau protein build up (termed tangles) inside the cells.

More specifically, “Beta-amyloid protein aggregations” as used herein relates to cerebral plaques laden with β-amyloid peptide (Aβ) and dystrophic neurites in neocortical terminal fields as well as prominent neurofibrillary tangles in medial temporal-lobe structures, which are important pathological features of Alzheimer's disease. Subsequently, loss of neurons and white matter, congophilic (amyloid) angiopathy are also present.

Aβ peptides are natural products of metabolism consisting of 36 to 43 amino acids. Monomers of Aβ40 are much more prevalent than the aggregation-prone and damaging Aβ42 species. β-amyloid peptides originate from proteolysis of the amyloid precursor protein by the sequential enzymatic actions of beta-site amyloid precursor protein-cleaving enzyme 1 (BACE-1), a β-secretase, and γ-secretase, a protein complex with presenilin 1 at its catalytic core. An imbalance between production and clearance, and aggregation of peptides, causes Aβ to accumulate, and this excess may be the initiating factor in Alzheimer's disease.

β-amyloid can also grow into fibrils, which arrange themselves into β-pleated sheets to form the insoluble fibers of advanced amyloid plaques. Soluble oligomers and intermediate amyloid are the most neurotoxic forms of Aβ. In brain-slice preparations, dimers and trimers of Aβ are toxic to synapses. Experimental evidence indicates that Aβ accumulation precedes and drives tau protein aggregation.

“Tau protein” as used herein, refers to neurofibrillary tangles, which are filamentous inclusions in pyramidal neurons, characteristic for Alzheimer's disease and other neurodegenerative disorders termed tauopathies. Elucidation of the mechanisms of their formation may provide targets for future therapies. Accumulation of hyperphosphorylated Tau protein as paired helical filaments in pyramidal neurons is a major hallmark of Alzheimer disease (AD). Besides hyperphosphorylation, other modifications of the Tau protein, such as cross-linking, are likely to contribute to the characteristic features of paired helical filaments, including their insolubility and resistance against proteolytic degradation. These neurofibrillary tangles, consist of hyperphosphorylated and aggregated forms of the microtubule-associated protein tau.

Under nonpathological conditions, tau is a developmentally regulated phosphoprotein that promotes assembly and stability of microtubules and is thus involved in axonal transport. In AD and other tauopathies, tau proteins aggregate and form fibrillar insoluble intracellular inclusions, so-called neurofibrillary tangles. It has been suggested that ionic interactions and covalent cross-linking contribute to pathological Tau aggregation and tangle formation. Reactive carbonyl compounds, which are increased under conditions of oxidative stress and in aging have been proposed as potential compounds responsible for tau aggregation.

“Alpha-synuclein pathology disorders” or “Synucleinopathies” is used to name a group of neurodegenerative disorders characterized by fibrillary aggregates of alpha-synuclein protein in the cytoplasm of selective populations of neurons and glia. More specifically, as used herein are disorders characterized by the presence of a specific intracellular protein aggregates (inclusion bodies) known as Lewy bodies that contain mainly alpha-synuclein protein. Alpha-synuclein protein is found naturally as an unfolded cytoplasmic protein in neuronal synaptic areas.

Overexpression of alpha-synuclein interrupts normal cell functions and leads to decreases in neurite outgrowth and cell adhesion. Alpha-synuclein aggregates comprising monomeric, oligomeric intermediate, or fibrillar forms are thought to be involved in a critical step in the pathogenesis of Parkinson's disease (PD) and in other alpha-synucleinopathies, such as multiple system atrophy (MSA) and dementia with Lewy bodies (DLB). These chronic neurodegenerative diseases of the CNS are characterized by the development of Lewy bodies containing alpha-synuclein protein. Oligomeric and monomeric alpha-synuclein have both been detected in cerebrospinal fluid and plasma samples from PD patients, suggesting that small aggregates of alpha-synuclein access the extracellular space. Previous animal and clinical data suggest that misfolded alpha-synuclein can be released from cells by exocytosis and transmitted from one brain area to another via cell-to cell propagation. Although the exact mechanism of alpha-synuclein transmission remains unknown, evidence suggests that clathrin-mediated endocytosis (CME) may have an important role in internalization of extracellular alpha-synuclein. As the cargo protein for endocytosis is usually recognized by a specific receptor on the cell surface, it is possible that alpha-synuclein may interact with cell-surface receptors that have not been well specified until now. N-methyl-D-aspartate (NMDA) receptor subunits contain motifs that bind the endocytic adaptor protein involved in CME. Additionally, a recent study provided the evidence that alpha-synuclein could promote endocytic internalization of surface NMDA receptors through a mechanism requiring clathrin, suggesting an interaction between alpha-synuclein and NMDA receptors. Accordingly, alpha-synuclein propagation from one area of the brain to others via cell-to-cell transmission is closely related with disease progression or clinical severity. Still further, Lewy body pathology in Parkinson's disease also found in peripheral nervous system. In neurons innervating the gastrointestinal tract and appendix. Peripheral Lewy pathology is suggested to precede the CNS Lewy pathology and according to Braak hypothesis, precede disease onset.

“Parkinson's disease (PD)” as used herein, is a neurodegenerative disease resulting from degeneration of midbrain dopamine neurons and accumulation of alpha-synuclein containing Lewy bodies in surviving neurons. The diagnosis of PD is based on the presence of cardinal motor features in the absence of other aetiological conditions. These motor features include the classical triad of bradykinesia, a resting pill-rolling tremor, and rigidity typically in association with hypomimia, hypophonia, micrographia and postural instability. Non-motor features of PD may even precede its diagnosis, constituting prodromal or premotor PD. These premotor features include problems with olfaction, constipation, mood and sleep, and following the clinical diagnosis of PD, they can become more prominent. Cognitive problems and dementia also commonly develop in PD, affecting almost 50% by 10 years from diagnosis. However, in some individuals with an alpha-synucleinopathy, significant cognitive problems precede the onset of parkinsonian motor symptoms, and these cases are clinically classified with a diagnosis of Dementia with Lewy Bodies. There is clearly a major degree of overlap between these two conditions both clinically and pathologically, but at present, the clinical distinction rests on the time interval between the onset of motor symptoms and dementia, with a minimum one year interval being required for a diagnosis of PD as opposed to Lewy body dementia (DLB).

Multiple system atrophy (MSA) is the rarest of the three major alpha synucleinopathies and differs significantly from PD and DLB in terms of its clinical presentation and its more aggressive course, reflecting differences in the underlying neuroanatomical pathways involved.

In yet some further embodiments, the invention may be applicable for DLB. More specifically, “Dementia with Lewy Bodies (DLB)”, as used herein, is a relatively common cause of dementia, estimated to account for up to 30% of dementia cases, and affecting up to 5% of those over the age of 75. Pathologically, it is defined by the presence of alpha synuclein containing Lewy bodies in the brain, but their distribution differs from that in PD, affecting the neocortex, limbic system and brainstem, in contrast to the nigrostriatal and brainstem-predominant pattern seen in early PD.

In yet some further embodiments, the invention may be applicable for MSA. “Multiple system atrophy (MSA)”, as used herein, is much rarer than PD with an estimated prevalence of 4.4 per 100 000 (PD is around 45 times more common).

As described herein above, the invention provides in some aspects thereof therapeutic and prophylactic methods. It is to be understood that the terms “treat”, “treating”, “treatment” or forms thereof, as used herein, mean preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease.

The term “treatment or prevention” as used herein, refers to the complete range of therapeutically positive effects of administrating to a subject including inhibition, reduction of, alleviation of, and relief from, an immune-related condition and illness, immune-related symptoms or undesired side effects or immune-related disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively. The term “amelioration” as referred to herein, relates to a decrease in the symptoms, and improvement in a subject's condition brought about by the compositions and methods according to the invention, wherein said improvement may be manifested in the forms of inhibition of pathologic processes associated with the immune-related disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with. The term “eliminate” relates to the substantial eradication or removal of the pathologic symptoms and possibly pathologic etiology, optionally, according to the methods of the invention described herein. The terms “delay”, “delaying the onset”, “retard” and all variations thereof are intended to encompass the slowing of the progress and/or exacerbation of a disorder associated with the immune-related disorders and their symptoms slowing their progress, further exacerbation or development, so as to appear later than in the absence of the treatment according to the invention. As indicated above, the methods and compositions provided by the present invention may be used for the treatment of a “pathological disorder”, specifically, immune-related disorders as specified by the invention, which refers to a condition, in which there is a disturbance of normal functioning, any abnormal condition of the body or mind that causes discomfort, dysfunction, or distress to the person affected or those in contact with that person. It should be noted that the terms “disease”, “disorder”, “condition” and “illness”, are equally used herein. It should be appreciated that any of the methods and compositions described by the invention may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms. The present invention relates to the treatment of subjects or patients, in need thereof. By “patient” or “subject in need” it is meant any organism who may be affected by the above-mentioned conditions, and to whom the therapeutic and prophylactic methods herein described are desired, including any vertebrate, specifically mammals such as humans, domestic and non-domestic mammals such as canine and feline subjects, bovine, simian, equine and rodents, specifically, murine subjects. More specifically, the methods of the invention are intended for mammals. By “mammalian subject” is meant any mammal for which the proposed therapy is desired, including human, livestock, equine, canine, and feline subjects, most specifically humans. It should be appreciated that the invention may be applicable for any vertebrates, for example, avian subjects, and fish.

Somatic hypermutation (SHM) occurs in response to antigen-dependent B cell activation in specialized lymphoid structures termed germinal centers (GCs). SHM introduces mainly point mutations into V exons. GC B cells with SHMs that result in increased BCR antigen-binding affinity are positively selected, leading to affinity maturation, and those that decrease BCR affinity or cause loss of BCR expression are negatively selected. Both V exon SHM and Class Switch Recombination are initiated by Activation-Induced Cytidine Deaminase (AID). AID acts as a mutagen by deamination of cytosine and converting it to uracil (C→U) in single-strand DNA, leading upon DNA replication to transition mutation replacing C by T and converting the C:G nucleotide pair into T:A pair (‘phase 1A’ mutations).

Deamination of C also triggers multiple pathways of base excision and mismatch repair that leads to the generation of somatic point mutations not only in the initial C/G target nucleotides but also in neighboring regions, including A/T nucleotides. Within the rearranged IgV-genes, predominant AID-targeting sites are targeted to the underlined C/G in certain hotspot motifs such as the WRCH/DGYW (with the palindromic AGCT motif representing a canonical example) and RCY/RGY as well as the WA/TW motifs. More specifically, W is any one of adenosine (A) or thymidine (T), R is A or guanosine (G), H is A or cytidine (C) or T, D is A or G or T and Y is C or T.

All scientific and technical terms used herein have meanings commonly used in the art unless otherwise specified. The definitions provided herein are to facilitate understanding of certain terms used frequently herein and are not meant to limit the scope of the present disclosure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. Thus, as used herein the term “about” refers to ±10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, and/or parts, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

### EXAMPLES

### Experimental Procedures

crRNA and sgRNA Design

For the gRNA targeting the mouse IgH locus, the previously published sequence (Nahmad et al. 2020 Nat. Comm.): CGATGCATAGGGACAAAGAGTGG, as denoted by SEQ ID NO: 1; (PAM sequence indicated in bold), was used.

For the gRNA targeting the human IgH locus, the following gRNAs are used (PAM indicated in bold):
GGAAAGAGAACTGTCGGAGTGGG, as denoted by SEQ ID NO: 2;
GGCTGGAAAGAGAACTGTCGGAGTGGGT, as denoted by SEQ ID NO: 3;
AGTGGGTGAATCCAGCCAGGAGG, as denoted by SEQ ID NO: 4; CCCTGCTCTCATCAAGACCGGGG, as denoted by SEQ ID NO: 5.

### Plasmid Cloning

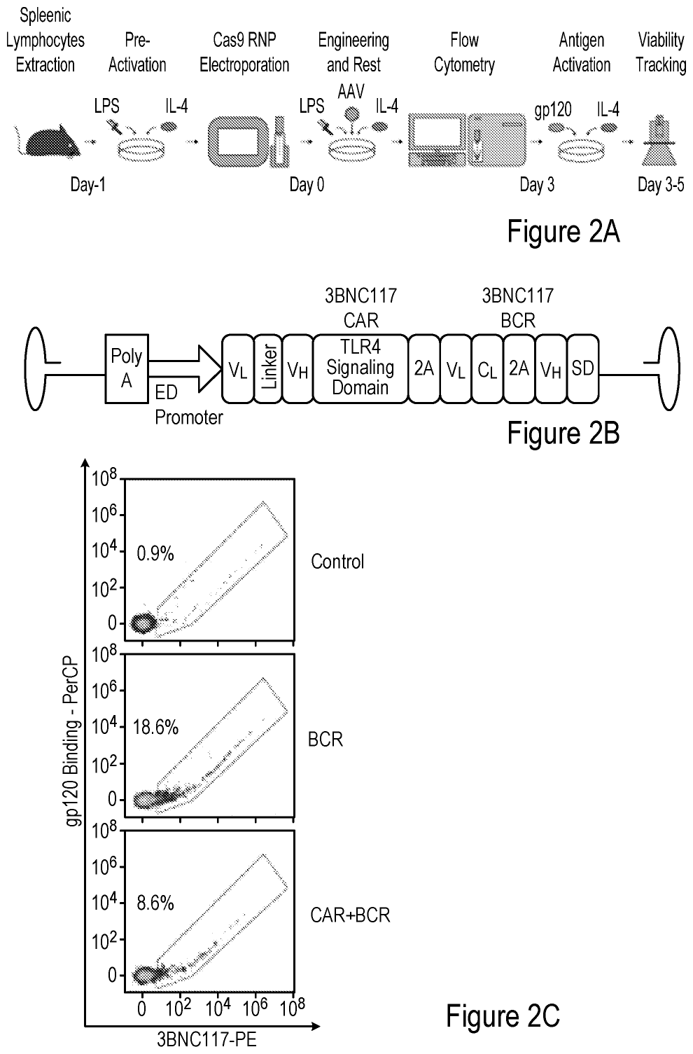

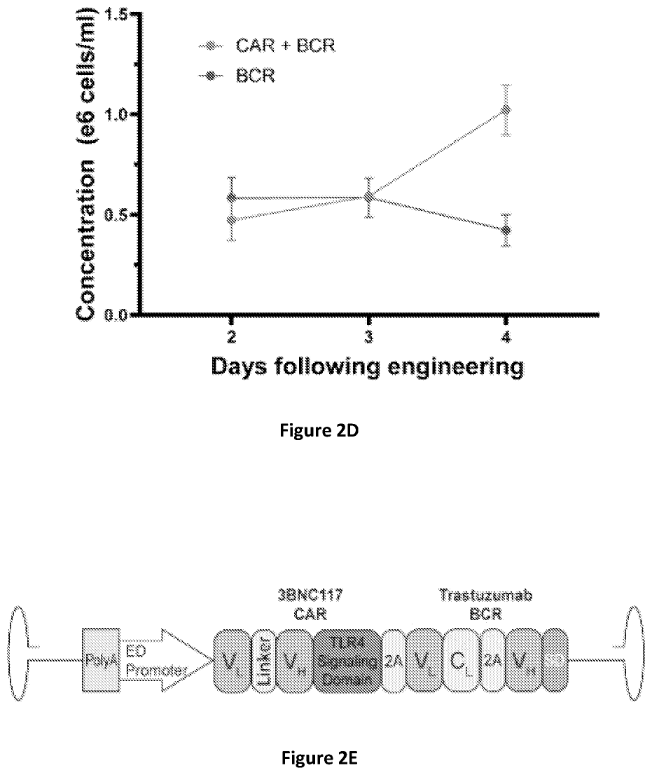

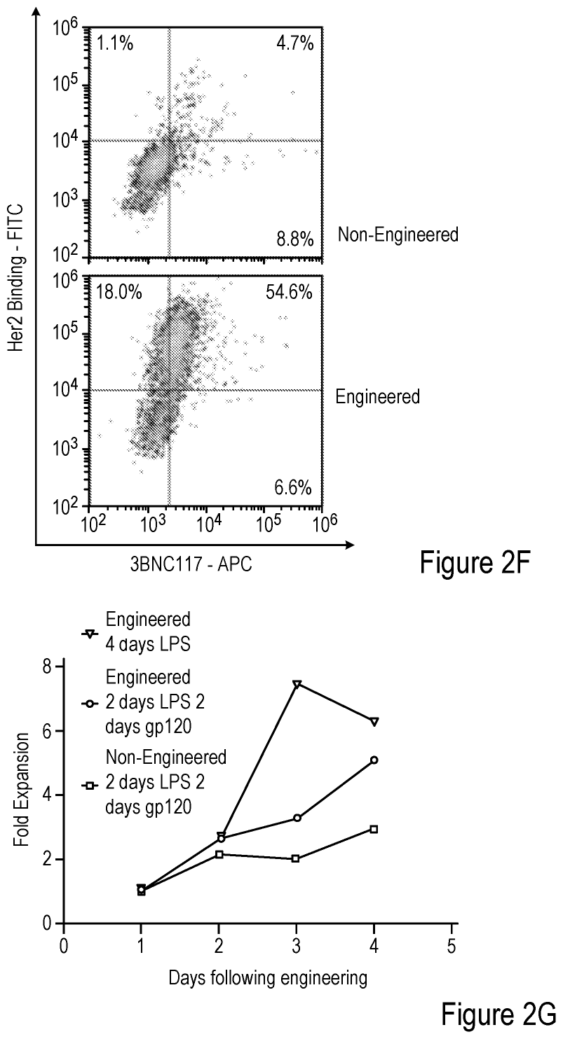

For the donor vectors, two intronic sequences directly adjacent to the mouse target gRNA (homology arms) were PCR amplified from the ImProB cell line (Nahmad et al. 2020 Nat. Comm). Fragments were inserted by In-Fusion assembly into pAB270 cut with NheI and SpeI. DNA sequences for the CAR and/or BCR were then inserted into the XhoI restriction site by HiFi DNA Assembly (NEB). Non-limiting examples for relevant sequences inserted as presented in FIG. 2, are described in Table 1, and denoted by the amino acid sequences as denoted by SEQ ID NOS: 12, 13, 14, 15, 16, or any derivatives thereof.

TABLE 1
CAR molecules

| CAR description | SEQ ID NO: |
|---|---|
| Trastuzumab CAR with TLR4 Signaling domain | 12 |
| Trastuzumab CAR with CD40 Signaling domain | 13 |
| 3BNC117 CAR with TLR4 Signaling domain | 14 |
| 3BNC117 CAR with CD40 Signaling domain | 15 |
| Trastuzumab CAR with CD40 and CD79b signaling domain | 16 |

rAAV Production and Purification

rAAV-DJ donors (Grimm, D. et al. J. Virol. 82, 5887-5911 (2008)) were produced in 293t cell lines by transient transfection. In short, 10-14 15 cm dishes were transfected when cells were 80% confluent pAd5 (helper plasmid), rAAVDJ or rAAV6 genome plasmid and Donor plasmid at a 3:1:1 ratio in Polyethylenimine (PEI)(Merck). In total each plate was transfected with 41,250 ng of DNA. Purification was performed with the AAVpro Extraction Kit (Takara) according to manufacturer protocol and titer quantification by qPCR with SYBRGreen (ThermoFisher).

### Generation of DNA Sequence for Trastuzumab Genetic Delivery

Variable light and heavy for the Trastuzumab part were reverse translated from pVITRO1-Trastuzumab-IgG1/k (Addgene plasmid #61883) and codon optimized for mouse.

From these sequences, splice donor and splice acceptor sites were removed that gave a score >0.6 using a splice prediction tool, by silent mutations.

### Generation of DNA Sequence for 3BNC117 Genetic Delivery

VRC01-class, gp120 CD4bs binding antibody 3BNC117 VH and VL were taken as from the published sequence (Accession numbers HE584538.1 and HE584537.1). Next, SHM hotspots (Laskov, R., et al. Mol. Immunol. 48, 733-745 (2011)) were added where possible, in the CDR loops. The same Furin-GSG-2A sequence as for the MEDI-493 was taken. The endogenous, natural leader peptides from the V sequences that generated the 3BNC117 VH and VL, (IgKV1D-33 and IgHV1-2) were added in their spliced from. From these sequences, splice donor and splice acceptor sites were removed that gave a score >0.6 using a splice prediction tool, by silent mutations. Finally, translation frame was controlled by generation of in-silico files mimicking genomic DNA insertion and mRNA splicing.

### Generation of DNA Sequence for CAR Genetic Delivery

Combinations of Trastuzumab or 3BNC117 variable light and heavy, separated by a 15mer G4S linker were assembled together with the 59mer linker preceding the transmembranal and cytoplasmic domain of the TLR4 or CD40.

Murine CD40 (TNR5) transmembranal and cytoplasmic domain was taken from the sequence as denoted by uniprot accession number P27512, and also denoted by SEQ ID NO: 6 and reverse translated and codon optimized for mouse. Murine TLR4 transmembranal and cytoplasmic domains were taken from the sequence as denoted by niport accession number Q9QUK6, and also denoted by SEQ ID NO: 7 and reverse translated and codon optimized for mouse. The amino acid sequence of the murine TLR4 transmembanal sequence is VIVVSTVAFLIYHFYFHLILI, as show in SEQ ID NO: 7 following the examples. Full sequences of the relevant sequences, inserted into the homology arms bearing vector can be found in Table 1.

### Primary Cells In-Vitro Culture and Activation

Whole spleens were extracted from 6-8 weeks old mice and tissue was mechanistically crushed in PBS to be filtered in 70 μm Cell Stainer (Corning). Following Red Blood Cell lysis (Biolegend) cells were plated at 3.0E6 cells/ml in 1640 RPMI supplemented with 10% HI FBS, 50 μM β-Mercaptoethanol, P/S, 10 μg/ml LPS (SantaCruz Biotechnology) and 10 ng/ml IL4 (Peprotech). Cells were cultured 16-24 h and washed twice in PBS before transfections and plated in the same activation medium without P/S after electroporation. Cells were cultured 16-24 h and washed in PBS before transfections and plated in the same activation medium without P/S for 8-16 h following electroporations. Parameters were 1350v 30 ms 1 pulse at 4.0E6 cells/10 μl in buffer R for 10 μl tips. For RNP, Cas9 (IDT) and gRNA (IDT) complexes assembly were generated 20 min prior of transfection with 18.3 pmol Cas9 66 pmol gRNA per 1E06 cells. Transductions were performed no later than 5 min following electroporation with 50,000 MOI AAV-DJ and cells were analyzed 2 days following electroporation for flow cytometry. Subsequent gp120 culture for co-activation was performed at a concentration of 10 μg/ml, in 1640 RPMI supplemented with 10% HI FBS, 50 μM β-Mercaptoethanol, P/S, 10 μg/ml LPS (SantaCruz Biotechnology) and 10 ng/ml IL4 (Peprotech).

### Mouse Studies

Mice studies were done in concordance with approval of Tel Aviv University Committee for the Use and Treatment of Laboratory Animals. 6-10 weeks old female C56/BL6OlaHsd mice were sacrificed for spleen harvest.

### Protein Expression and Purification

For Env proteins, plasmid encoding His-tagged gp140YU2 was transfected into Expi293F cells at a density of 2.0E6 cells/ml in Expi293 Expression Medium (ThermoFisher) using ExpiFectamine (ThermoFisher) according to manufacturer protocols. Supernatants were collected 7 days later and bound with Ni-NTA Agarose (Qiagen) in 20 mM Sodium Phosphate, 0.5M NaCl, 10 mM Imidazol (ID). Beads were washed twice with the same buffer before mounting on gravity-flow Polypropylene columns (Biorad). Chromatography elution was performed in three fractions: 50, 100 and 200 mM ID. Elutes were buffer exchanged to PBS using Amicon Ultra-15 Centrifugal Filter Units (Merck). For anti-3BNC117, 7.5 μg of plasmid encoding the light chain pADN210 and 22.5 μg of plasmid encoding heavy chain pADN211 were co-trasfected into 1 30 mL flask of Expi293F cells at a density of 2.0E6 cells/ml in Expi293 Expression Medium (ThermoFisher) using ExpiFectamine (ThermoFisher) according to manufacturer protocols. Purification of the supernatant, 7 days post transfection, was performed using MabSelect (GE Healthcare) following manufacturer recommendations.

### Flow Cytometry

Harvested cells were resuspended in cell staining buffer (Biolegend) and incubated with 1 μg/100 μl of human anti-3BNC117 and/or gp120 and/or Her2 for 10 mins, washed and resuspended again in cell staining buffer containing secondary antibodies. Secondary staining was performed in the dark, for 15 mins, with anti-human IgG-PE (Abcam) or anti-human IgG AF647 (abcam) and anti His-tag PerCP (Biolegend) or anti-HisTag AF488 (MBL). Then, cells were washed and data acquisition was performed on a CytoFLEX (Beckman Coulter) or Attune Nxt (life technologies) for experiments involving cell sorting. Data were compiled and analyzed using Kaluza Analysis 2.1 (Beckman Coulter).

### Example 1

### Design of 3BNC117 CAR B Cells Engineered for T Cell Independent Activation

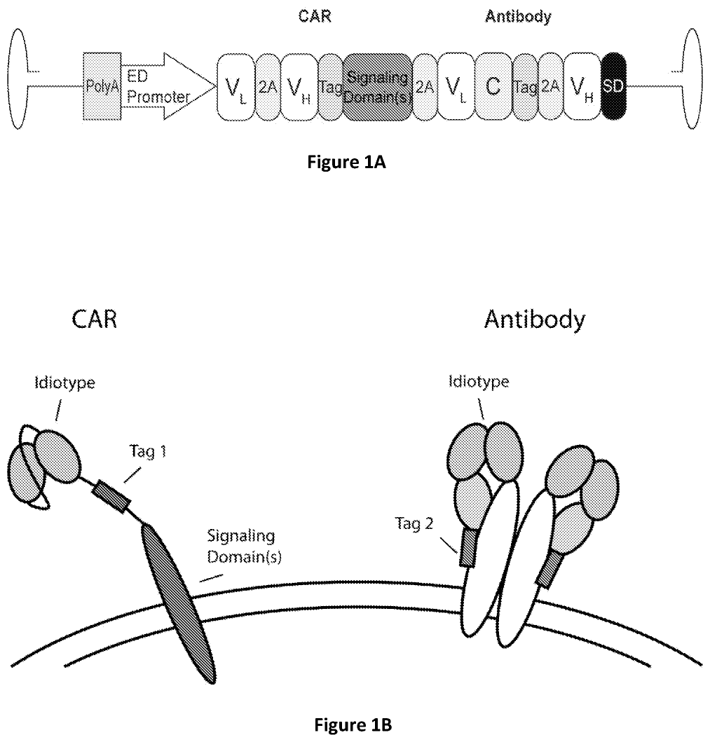

The inventors designed a CAR molecule comprising the scfv domain of an antibody, either 3BNC117 or Trastuzumab, a Strep-II tag, and a single or double signaling domains. The signaling domains used herein are selected from: TLR4, CD40, IL21R, BAFF-R, Fas and IFNγR. In some embodiments, the CAR gene is encoded upstream to a cassette encoding a B cell receptor. The B cell receptor cassette includes a full antibody light chain as well as the variable segment of the antibody's heavy chain (VH) separated by a furin cleavage site and a 2A-peptide for ribosomal skipping. The VH is followed by a splice donor sequence to allow fusion to constant segments and initial expression of the antibody as a membranal B cell receptor (BCR). The B cell receptor has an HA tag. (FIG. 1A). The two antigen specific receptor molecules are expressed in the same engineered B cell (FIG. 1B).

### Example 2

### Engineering and Ex-Vivo Activation of Mouse B Cells Expressing 3BNC117 CAR and a BCR Based on 3BNC117 or Trastuzumab

Primary mouse splenic lymphocytes were engineered ex vivo to express both a CAR and a BCR (FIG. 2A). In particular, splenic B lymphocytes were collected from donor mice and activated with LPS and IL4. The following day, cells were engineered with CRISPR-Cas9 RNPs and AAV donors. Cells are cultured in the presence of LPS for two additional days before staining for flow cytometry. The inserted CAR gene is based on “3BNC117”, an anti-HIV gp120 antibody, and the inserted BCR gene is based on 3BNC117 (FIGS. 2B-2D) or on “trastuzumab”, an anti Her2 antibody (FIGS. 2E-2G). Expression of the 3BNC117 based CAR and BCR was detected by flow cytometry using the gp120 antigen and an anti-idiotypic antibody (FIGS. 2C and 2F) and the Her2 antigen (FIG. 2F). Finally, the culture is supplemented with gp120 for activation, and concentration (FIG. 2D) or fold expansion (FIG. 2G) is monitored for two additional days.

### Example 3

### Engineering and Ex-Vivo Activation of Human B Cells Expressing 3BNC117 CAR and BCR

Human PBMC derived B cells are engineered ex vivo to express differentially tagged anti-Her2 CAR and BCR, based on the sequence of the monoclonal antibody Trastuzumab. In particular, the CAR has a strep-II tag and the BCR has an HA tag. Antibodies toward each tag allow the monitoring of engineering efficiency.

To monitor T cell independent activation ex vivo, the engineered cells, expressing the Trastuzumab-based CAR+BCR are co-cultured with mouse cells, engineered to express the human Her2 antigen. Proliferation is monitored following engineering and culturing. In addition, the supernatant of the culture is collected each day following engineering for Trastuzumab specific ELISA. Finally, the CD138, CD38, GL-7, CD80, IgM, IgG markers are tracked by Flow Cytometry. For all experiments, inventors compare engineered cells cultured with Her2 to engineered cells cultured with gp120.

### Example 4

Adoptive Transfer of CAR Engineered B Cells into Immunocompetent Mice Pre-Implanted with a Her2 Expressing Cancer

The in vivo effect of CAR B cells engineered as in Example 3, in the immunocompetent setting is monitored upon systemic adoptive transfer into syngeneic transgenic mice expressing Her2 and bearing subcutaneous or orthotopic mammary E0771E2 tumors. In different mouse groups, the tumor or the B cells express luciferase, and treatment combinations with radiotherapy and checkpoint inhibition are evaluated. Factors monitored include survival, weight, appearance and behavior as well as tumor size and metastases. Antibody titers in the blood are further assessed. Upon sacrificing the mice, the abundance, isotype and immunopenotype of infiltrating CAR B cells and endogenous T cells are evaluated in the cancer and lymphatic tissues as well as the presence of antibody secreting cells in the tumor and in the BM. To confirm safety, autoimmune manifestations and cytokine storms are monitored. Off-target effects, secondary malignancies and genotoxicity are assessed. Next generation sequencing is further used to assess the off-target integration profile, and FISH and karyotyping are used to detect chromosomal aberrations. Finally, a safety switches based on either an inducible caspase or anti-CD20 antibodies, are developed to allow the fast and specific elimination of engineered B cells, or total B cells, respectively, if expression is found to be associated with adverse outcomes.

### Example 5

Adoptive Transfer of CAR Engineered B Cells into Humanized Mice Pre-Implanted with a Her2 Expressing Cancer

The in vivo activity of B cells from human PBMCs, engineered to express anti-Her2 CAR and BCR, is monitored following transplantation into NSG mice immune-reconstituted with autologous PBMCs and orthotopically transplanted with SK-BR-3 Her2-expressing human breast cancer cells. In different mouse groups, the tumor or the B cells express luciferase, and treatment combinations with radiotherapy and checkpoint inhibition are evaluated. Factors monitored include survival, weight, appearance and behavior as well as tumor size and metastases. Antibody titers in the blood are further assessed. Upon sacrificing the mice, the abundance, isotype and immunophenotype of infiltrating CAR B cells and endogenous T cells are evaluated in the cancer and lymphatic tissues as well as the presence of antibody secreting cells in the tumor and in the BM. To confirm safety, autoimmune manifestations and cytokine storms are monitored. Off-target effects, secondary malignancies and genotoxicity are assessed. We further use next generation sequencing to assess the off-target integration profile and use FISH and karyotyping to detect chromosomal aberrations. Finally, safety switches based on either an inducible caspase or anti-CD20 antibodies are developed to allow the fast and specific elimination of engineered B cells, or total B cells, respectively, if expression is found to be associated with adverse outcomes.

### SEQUENCE LISTING

### SEQ ID NO: 1

### DNA

### Artificial

gRNA targeting the mouse IgH locus
CGATGCATAGGGACAAAGAGTGG, as denoted by SEQ ID NO: 1

### SEQ ID NO: 2

### DNA

### Artificial

gRNA targeting the human IgH locus

GGAAAGAGAACTGTCGGAGTGGG

### SEQ ID NO: 3

### DNA

### Artificial

gRNA targeting the human IgH locus

GGCTGGAAAGAGAACTGTCGGAGTGGGT

### SEQ ID NO: 4

### DNA

### Artificial

gRNA targeting the human IgH locus

AGTGGGTGAATCCAGCCAGGAGG

### SEQ ID NO: 5

### DNA

### Artificial

gRNA targeting the human IgH locus

CCCTGCTCTCATCAAGACCGGGG

### SEQ ID NO: 6

### Protein

### Murine CD40 (TNR5) Transmembranal and Cytoplasmic Domain Uniprot P27512

ALLVIPVVMGILITIFGVFLYIKKVVKKPKDNEILPPAARRQDPQEMED
YPGHNTAAPVQETLHGCQPVTQEDGKESRISVQERQVTDSIALRPLV

Transmembrane domain is indicated in bold.

### SEQ ID NO: 7

### Protein

### Murine TLR4 Transmembranal and Cytoplasmic Domains Niport Q9QUK6

VIVVSTVAFLIYHFYFHLILIAGCKKYSRGESIYDAFVIYSSQNEDWVR
NELVKNLEEGVPRFHLCLHYRDFIPGVAIAANIIQEGFHKSRKVIVVVS
RHFIQSRWCIFEYEIAQTWQFLSSRSGIIFIVLEKVEKSLLRQQVELYR
LLSRNTYLEWEDNPLGRHIFWRRLKNALLDGKASNPEQTAEEEQETATW
T

Transmembrane domain is indicated in bold.

### SEQ ID NO: 8

### Protein

### VH of Trastuzumab

EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSS

### SEQ ID NO: 9

### Protein

### VL of Trastuzumab

DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKLEIKRTVAAP

### SEQ ID NO: 10

### Protein

### VH of the 3BNC117

QVQLLQSGAAVTKPGASVRVSCEASGYNIRDYFIHWWRQAPGQGLQWVG
WINPKTGQPNNPRQFQGRVSLTRHASWDFDTFSFYMDLKALRSDDTAVY
FCARQRSDYWDFDVWGSGTQVTVSS

### SEQ ID NO: 11

### Protein

### VL of the 3BNC117

DIQMTQSPSSLSASVGDTVTITCQANGYLNWYQQRRGKAPKLLIYDGSK
LERGVPSRFSGRRWGQEYNLTINNLQPEDIATYFCQVYEFVVPGTRLDL
K

### SEQ ID NO: 12

### Protein

Trastuzumab CAR with TLR4 Signaling Domain

DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKLEIKRTVAAPGGGGSGGGGSGGGGSEVQLVESGGGLVQPGGSLR
LSCAASGFNIKDTYIHWVRQAPGKGLEWVARIYPTNGYTRYADSVKGRF
TISADTSKNTAYLQMNSLRAEDTAVYYCSRWGGDGFYAMDYWGQGTLVT
VSSGAGSGASWSHPQFEKGASGGSGGASWSHPQFEKGASGGSGGSGGAS
WSHPQFEKGASGGVIVVSTVAFLIYHFYFHLILIAGCKKYSRGESIYDA
FVIYSSQNEDWVRNELVKNLEEGVPRFHLCLHYRDFIPGVAIAANIIQE
GFHKSRKVIVVVSRHFIQSRWCIFEYEIAQTWQFLSSRSGIIFIVLEKV
EKSLLRQQVELYRLLSRNTYLEWEDNPLGRHIFWRRLKNALLDGKASNP
EQTAEEEQETATWT

Transmembrane domain is indicated in bold.

### SEQ ID NO: 13

### Protein

Trastuzumab CAR with CD40 Signaling Domain

DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKLEIKRTVAAPGGGGSGGGGSGGGGSEVQLVESGGGLVQPGGSLR
LSCAASGFNIKDTYIHWVRQAPGKGLEWVARIYPTNGYTRYADSVKGRF
TISADTSKNTAYLQMNSLRAEDTAVYYCSRWGGDGFYAMDYWGQGTLVT
VSSGAGSGASWSHPQFEKGASGGSGGASWSHPQFEKGASGGSGGSGGAS
WSHPQFEKGASGGALLVIPVVMGILITIFGVFLYIKKVVKKPKDNEILP
PAARRQDPQEMEDYPGHNTAAPVQETLHGCQPVTQEDGKESRISVQERQ
VTDSIALRPLV

Transmembrane domain is indicated in bold.

### SEQ ID NO: 14

### Protein

3BNC117 CAR with TLR4 Signaling Domain

DIQMTQSPSSLSASVGDTVTITCQANGYLNWYQQRRGKAPKLLIYDGSK
LERGVPSRFSGRRWGQEYNLTINNLQPEDIATYFCQVYEFVVPGTRLDL
KRGGGGSGGGGSGGGGSQVQLLQSGAAVTKPGASVRVSCEASGYNIRDY
FIHWWRQAPGQGLQWVGWINPKTGQPNNPRQFQGRVSLTRHASWDFDTF
SFYMDLKALRSDDTAVYFCARQRSDYWDFDVWGSGTQVTVSSGAGSGAS
WSHPQFEKGASGGSGGASWSHPQFEKGASGGSGGSGGASWSHPQFEKGA
SGGVIVVSTVAFLIYHFYFHLILIAGCKKYSRGESIYDAFVIYSSQNED
WVRNELVKNLEEGVPRFHLCLHYRDFIPGVAIAANIIQEGFHKSRKVIV
VVSRHFIQSRWCIFEYEIAQTWQFLSSRSGIIFIVLEKVEKSLLRQQVE
LYRLLSRNTYLEWEDNPLGRHIFWRRLKNALLDGKASNPEQTAEEEQET
ATWT

Transmembrane domain is indicated in bold.

### SEQ ID NO: 15

### Protein

3BNC117 CAR with CD40 Signaling domain

DIQMTQSPSSLSASVGDTVTITCQANGYLNWYQQRRGKAPKLLIYDGSK
LERGVPSRFSGRRWGQEYNLTINNLQPEDIATYFCQVYEFVVPGTRLDL
KRGGGGSGGGGSGGGGSQVQLLQSGAAVTKPGASVRVSCEASGYNIRDY
FIHWWRQAPGQGLQWVGWINPKTGQPNNPRQFQGRVSLTRHASWDFDTF
SFYMDLKALRSDDTAVYFCARQRSDYWDFDVWGSGTQVTVSSGAGSGAS
WSHPQFEKGASGGSGGASWSHPQFEKGASGGSGGSGGASWSHPQFEKGA
SGGALLVIPVVMGILITIFGVFLYIKKVVKKPKDNEILPPAARRQDPQE
MEDYPGHNTAAPVQETLHGCQPVTQEDGKESRISVQERQVTDSIALRPL
V

Transmembrane domain is indicated in bold.

### SEQ ID NO: 16

### Protein

Trastuzumab CAR with CD40 and CD79b Signaling Domain

DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKLEIKRTVAAPGGGGSGGGGSGGGGSEVQLVESGGGLVQPGGSLR
LSCAASGFNIKDTYIHWVRQAPGKGLEWVARIYPTNGYTRYADSVKGRF
TISADTSKNTAYLQMNSLRAEDTAVYYCSRWGGDGFYAMDYWGQGTLVT
VSSGAGSGASWSHPQFEKGASGGSGGASWSHPQFEKGASGGSGGSGGAS
WSHPQFEKGASGGALLVIPVVMGILITIFGVFLYIKKVVKKPKDNEILP
PAARRQDPQEMEDYPGHNTAAPVQETLHGCQPVTQEDGKESRISVQERQ
VTDSIALRPLVDKDDGKAGMEEDHTYEGLNIDQTATYEDIVTLRTGEVK
WSVGEHPGQE

Transmembrane domain is indicated in bold.

What is claimed is:

1. A chimeric antigen receptor (CAR) that allows a cell of the B cell lineage to undergo antigen-induced activation independent of a cell of the T cell lineage, wherein said CAR comprises:
(i) at least one target-binding domain comprising at least one target-recognition element, wherein said target is at least one antigen associated with a pathologic disorder and said pathologic disorder is at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition;
(ii) at least one transmembrane domain; and
(iii) at least one signal transduction domain, wherein said signal transduction domain is not derived from a B cell receptor and is capable of relaying signals for T cell independent activation; and
wherein said cell of the B cell lineage is at least one of B cell precursor, naïve B cell, and activated B cell of all phenotypes.

2. The CAR according to claim 1, wherein said at least one target-binding domain further comprises at least one adaptor component that recognizes and binds at least one tagged target-recognition element.

3. The CAR according to claim 1,
wherein said proliferative disorder is cancer and said target-binding domain specifically recognizes at least one tumor associated antigen (TAA), and said TAA is the human epidermal growth factor receptor 2 (HER2/neu) or any fragment thereof; or
wherein said pathogen is the human immunodeficiency virus (HIV) and said target-binding domain specifically recognizes a gp120 HIV antigen.

4. The CAR according to claim 3, wherein when said disorder is cancer, said target-recognition element comprises at least one antibody or any antigen-binding fragment/s, portion/s or chimera/s thereof that specifically recognizes and binds HER2/neu; or wherein when said pathogen is HIV, said target-recognition element comprises at least one antibody or any antigen-binding fragment/s, portion/s or chimera/s thereof that specifically recognizes and binds gp120.

5. The CAR according to claim 1, wherein said signal transduction domain is at least one intracellular co-stimulatory domain that induces T cell independent B cell activation, said intracellular co-stimulatory signal transduction domain comprising at least one of: (i) Cluster of differentiation 40 (CD40), (ii) Toll-like receptor 4 (TLR4), (iii) Inducible co-stimulatory ligand (ICOSL), (iv) Cluster of differentiation 80/86 (CD80/CD86), (v) interleukin-2 receptor (IL2R), (vi) interleukin-4 receptor (IL4R), (vii) interleukin-5 receptor (IL5R), (viii) interleukin-6 receptor (IL6R), (ix) interleukin-10 receptor (IL10R), (x) interleukin-12 receptor (IL12R), (xi) interleukin-13 receptor (IL13R), (xii) interleukin-21 receptor (IL21R), (xiii) Transmembrane activator and CAML interactor (TACI), (xiv) B-cell maturation antigen (BCMA), (xv) B-cell activating factor receptor (BAFF-R), (xvi) Major histocompatibility complex class II (MHCII), or any combinations thereof.

6. A nucleic acid molecule comprising at least one nucleic acid sequence encoding at least one CAR that allows a cell of the B cell lineage to undergo antigen-induced activation independent of a cell of the T cell lineage, or any cassette, vector or vehicle comprising said nucleic acid molecule, said nucleic acid sequence encoding said at least one CAR encoding 5′ to 3′ (i) a target-recognition element encoding an at least one antibody or any antigen-binding fragment/s, portion/s or chimera/s thereof that specifically recognizes an at least one antigen associated with a pathologic disorder, said pathologic disorder at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition; (ii) at least one transmembrane domain; and (iii) at least one signal transduction domain, wherein said signal transduction domain is not derived from a B cell receptor and is capable of relaying signals for T cell independent activation; wherein said nucleic acid molecule is optionally flanked on the 5′ and 3′ ends by homology arms for integration to a target site by homologous recombination; or is optionally flanked on at least one of the 5′ and 3′ ends thereof by at least one of: recognition sites for a site-specific nuclease, a site-specific integrase, or a site-specific recombinase.

7. The nucleic acid molecule of claim 6, wherein said nucleic acid sequence encoding said target recognition sequence encodes 5′ to 3′a variable light chain region and a variable heavy chain region that specifically recognize at least one antigen associated with a proliferative disorder, said disorder being cancer and said antigen being HER2, or specifically recognize at least one antigen associated with an infectious disease caused by a pathogen, said pathogen being HIV and said antigen being gp120.

8. The nucleic acid molecule of claim 6, further comprising a nucleic acid sequence upstream of said at least one nucleic acid sequence encoding said CAR and a nucleic acid sequence downstream of said at least one nucleic acid sequence encoding said CAR, said upstream nucleic acid sequence encoding 5′ to 3′, a poly-adenylation site and an enhancer dependent promoter, and said downstream nucleic acid sequence encoding 5′ to 3′, at least one membranal and/or secreted antibody or antibody derivative, and a splice donor site.

9. The nucleic acid of claim 8, wherein said at least one membranal and/or secreted antibody or antibody derivative specifically recognizes and binds HER2 or gp120.

10. A gene editing system comprising:
(i) at least one nucleic acids molecule of claim 6, or any cassette, vector or vehicle comprising said at least one nucleic acid molecule; and
(ii) at least one gene editing component or a nucleic acid sequence encoding said gene editing component.

11. A genetically engineered cell of the B cell lineage expressing at least one CAR molecule, or any population of cells comprising at least one said genetically modified cell of the B cell lineage, wherein said cell is capable of undergoing antigen-induced activation independent of a cell of the T cell lineage, and wherein said CAR comprises:
(i) at least one target-binding domain comprising at least one target-recognition element, wherein said target is at least one antigen associated with a pathologic disorder and said pathologic disorder is at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition;
(ii) at least one transmembrane domain; and
(iii) at least one signal transduction domain, wherein said signal transduction domain is not derived from a B cell receptor and is capable of relaying signals for T cell independent activation; and
wherein said cell of the B cell lineage is at least one of B cell precursor, naïve B cell, and activated B cell of all phenotypes.

12. The genetically engineered cell according to claim 11,
(a) wherein when said target is associated with said proliferative disorder, said proliferative disorder is cancer, and said target-binding domain specifically recognizes at least one tumor associated antigen (TAA), wherein optionally, said TAA is the human epidermal growth factor receptor 2 (HER2/neu) or any fragment thereof and said target-recognition element comprises at least one antibody or any antigen-binding fragment/s, portion/s or chimera/s thereof that specifically recognizes and binds HER2/neu; or
(b) wherein when said target is associated with an infectious disease, said pathogen is the human immunodeficiency virus (HIV) and said target-recognition element comprises at least one antibody or any antigen-binding fragment/s, portion/s or chimera/s thereof that specifically recognizes and binds gp120.

13. The genetically engineered cell according to claim 11, wherein said signal transduction domain is at least one intracellular co-stimulatory domain that induces T cell independent B cell activation, and said intracellular co-stimulatory signal transduction domain comprises at least one of: (i) Cluster of differentiation 40 (CD40), (ii) Toll-like receptor 4 (TLR4), (iii) Inducible co-stimulatory ligand (ICOSL), (iv) Cluster of differentiation 80/86 (CD80/CD86), (v) interleukin-2 receptor (IL2R), (vi) interleukin-4 receptor (IL4R), (vii) interleukin-5 receptor (IL5R), (viii) interleukin-6 receptor (IL6R), (ix) interleukin-10 receptor (IL10R), (x) interleukin-12 receptor (IL12R), (xi) interleukin-13 receptor (IL13R), (xii) interleukin-21 receptor (IL21R), (xiii) Transmembrane activator and CAML interactor (TACI), (xiv) B-cell maturation antigen (BCMA), (xv) B-cell activating factor receptor (BAFF-R), Major histocompatibility complex class II (MHCII), and any combinations thereof.

14. The genetically engineered cell according to claim 11, wherein said cell further expresses and or secretes at least one effector protein, said effector protein is an at least one membranal and/or secreted antibody or antibody derivative.

15. A pharmaceutical composition comprising:
at least one CAR that allows a cell of the B cell lineage to undergo antigen-induced activation independent of a cell of the T cell lineage, or any nucleic acid molecule comprising at least one nucleic acid sequence encoding said CAR, or any, cassette, vector, vehicle, or gene editing system comprising said nucleic acid molecule, any cell expressing said CAR, or any genetically engineered B cell expressing said CAR or population of cells comprising at least one said genetically engineered B cell, wherein said nucleic acid sequence encoding said at least one CAR encodes 5′ to 3′ (i) a target-recognition element encoding an at least one antibody or any antigen-binding fragment/s, portion/s or chimera/s thereof that specifically recognizes an at least one antigen associated with a pathologic disorder, said pathologic disorder at least one of:
a proliferative disorder, an inflammatory disorder, an infectious disease caused by a pathogen, an autoimmune disease, a neurodegenerative disease, a congenital disorder, an allergic condition, a cardiovascular disease, and a metabolic condition; (ii) at least one transmembrane domain; and (iii) at least one signal transduction domain, wherein said signal transduction domain is not derived from a B cell receptor and is capable of relaying signals for T cell independent activation; wherein said nucleic acid molecule is optionally flanked on the 5′ and 3′ ends by homology arms for integration to a target site by homologous recombination; or is optionally flanked on at least one of the 5′ and 3′ ends thereof by at least one of: recognition sites for a site-specific nuclease, a site-specific integrase, or a site-specific recombinase; and
at least one pharmaceutically acceptable carrier/s, diluent/s, excipient/s, or additive/s, or a combination thereof.

16. A method for genetic engineering of a cell of the B cell lineage to express at least one CAR molecule that allows said cell to undergo antigen-induced activation independent of a cell of the T cell lineage, the method comprising the step of contacting said cell with an effective amount of at least one nucleic acid molecule comprising at least one nucleic acid sequence encoding said CAR or a system comprising said CAR, or any cassette, vehicle, vector, or gene editing system comprising said nucleic acid molecule, or with a composition thereof, wherein said nucleic acid sequence encoding CAR encodes 5′ to 3′ (i) a target-recognition element encoding an at least one antibody or any antigen-binding fragment/s, portion/s or chimera/s thereof that specifically recognizes an at least one antigen associated with a pathologic disorder, said pathologic disorder at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition; (ii) at least one transmembrane domain; and (iii) at least one signal transduction domain, wherein said signal transduction domain is not derived from a B cell receptor and is capable of relaying signals for T cell independent activation; wherein said nucleic acid molecule is optionally flanked on the 5′ and 3′ ends by homology arms for integration to a target site by homologous recombination; or is optionally flanked on at least one of the 5′ and 3′ ends thereof by at least one of: recognition sites for a site-specific nuclease, a site-specific integrase, or a site-specific recombinase; and
wherein said cell of the B cell lineage is at least one of B cell precursor, naïve B cell, and activated B cell of all phenotypes.

17. The method according to claim 16, wherein said cell is engineered to further express at least one effector molecule, wherein said effector protein is an at least one membranal and/or a secreted antibody or antibody derivative.

18. A method for treating, preventing, ameliorating, inhibiting or delaying the onset of a pathologic disorder in a mammalian subject, said method comprising the step of administering to said subject an effective amount of at least one of:
(a) at least one nucleic acid molecule encoding at least one CAR that allows a cell of the B cell lineage to undergo antigen-induced activation independent of a cell of the T cell lineage or encoding at least one system comprising said CAR;
(b) at least one cassette, vector vehicle, or gene editing system comprising said nucleic acid molecule of (a);
(c) at least one cell expressing said CAR or a system comprising said CAR, or a population of said cells; or
(d) a composition comprising at least one of (a), (b) and (c);
wherein said CAR comprises:
(i) at least one target-binding domain comprising at least one target-recognition element, wherein said target is at least one antigen associated with a pathologic disorder and said pathologic disorder is at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition;
(ii) at least one transmembrane domain; and
(iii) at least one signal transduction domain, wherein said signal transduction domain is not derived from a B cell receptor and is capable of relaying signals for T cell independent activation; and
wherein said cell of the B cell lineage is at least one of B cell precursor, naïve B cell, and activated B cell of all phenotypes.

19. The method according to claim 18, wherein said proliferative disorder is cancer, and wherein said target-binding domain of said CAR specifically recognizes at least one tumor associated antigen (TAA), and wherein optionally said TAA is the human epidermal growth factor receptor 2 (HER2/neu) or any fragment thereof and said target-recognition element of said CAR comprises at least one antibody or any antigen-binding fragment/s, portion/s or chimera/s thereof that specifically recognizes and binds HER2/neu.

20. The method according to claim 18, wherein said pathogen is the human immunodeficiency virus (HIV) and said target-recognition element of said CAR comprises at least one antibody or any antigen-binding fragment/s, portion/s or chimera/s thereof that specifically recognizes and binds gp120.
